(19) United States
(12) Patent Application Publication
(10) Pub. No.: US 2024/0003918 A1
(43) Pub. Date: Jan. 4, 2024

(54) NON-INVASIVE ASSESSMENT OF ALZHEIMER'S DISEASE

(71) Applicants: Michael Reitermann, Toronto (CA); Thomas H. Tulip, Toronto (CA); Mathotaarachchilage Sulantha Sanjeewa Mathotaarachchi, Toronto (CA); ENIGMA BIOINTELLIGENCE, INC., Toronto (CA)

(72) Inventors: Michael Reitermann, Nantucket, MA (US); Thomas H. Tulip, Andover, MA (US); Mathotaarachchilage Sulantha Sanjeewa Mathotaarachchi, Vancouver (CA)

(73) Assignee: ENIGMA BIOINTELLIGENCE, INC., Toronto, ON (CA)

(21) Appl. No.: 18/254,438

(22) PCT Filed: Nov. 29, 2021
(86) PCT No.: PCT/US2021/061016

§ 371 Date: May 25, 2023

Related U.S. Application Data
(60) Provisional application No. 63/119,372, filed on Nov. 30, 2020.

(51) Int. Cl. G01N 33/68; G16H 50/30; G16H 50/20

(52) U.S. Cl. CPC G01N 33/6896; G16H 50/30; G16H 50/20

(57) ABSTRACT

The present disclosure generally to non-invasive methods and tests that measure biomarkers and collect clinical parameters from subjects, and computer-implemented processes for assessing a likelihood that a patient has or will develop Alzheimer's Disease, e.g., by assigning the subject an Alzheimer's Disease risk score.

### 1. CROSS-REFERENCE TO RELATED APPLICATIONS

This application claims the priority benefit of U.S. provisional application No. 63/119,372, filed Nov. 30, 2020, the contents of which are incorporated herein in their entireties by reference thereto.

### 2. BACKGROUND

Alzheimer's disease (AD) is a devastating neurodegenerative disease and the predominant form of dementia (50-75%). In 2015, ˜44 million people worldwide were estimated to have AD or a related dementia and each year, 4.6 million new cases of dementia are predicted (Van Cauwenberghe et al., 2016, Genetics in Medicine 18: 421-430). Two pathological characteristics are observed in AD patients at autopsy: extracellular plaques and intracellular tangles in the hippocampus, cerebral cortex, and other areas of the brain essential for cognitive function. Plaques are formed mostly from the deposition of amyloid beta (“Aβ”), a peptide derived from amyloid precursor protein (“APP”). Filamentous tangles are formed from paired helical filaments composed of neurofilament and hyperphosphorylated tau protein, a microtubule-associated protein. It is not clear, however, whether these two pathological changes are only associated with the disease or truly involved in the degenerative process. Late-onset/sporadic AD has virtually identical pathology to inherited early-onset/familial AD (FAD), thus suggesting common pathogenic pathways for both forms of AD. To date, genetic studies have identified three genes that cause autosomal dominant, early-onset AD, amyloid precursor protein (“APP”), presenilin 1 (“PS1”), and presenilin 2 (“PS2”). A fourth gene, apolipoprotein E (“APOE or APO E”), is the strongest and most common genetic risk factor for AD, but does not necessarily cause it. All mutations associated with APP and PS proteins can lead to an increase in the production of Aβ peptides, specifically the more amyloidogenic form, Aβ42. In addition to genetic influences on amyloid plaque and intracellular tangle formation, environmental factors (e.g., cytokines, neurotoxins, etc.) may also play important roles in the development and progression of AD.

The disease is clinically characterized by progressive deterioration of memory and cognitive functions, leading to loss of autonomy and ultimately requiring full-time medical care. Besides the strong impact of AD on the patient and primary caregivers, there is an enormous burden on society and public health due to the high costs associated with care and treatment of dementia. Aside from drugs that temporarily relieve symptoms, no FDA-approved treatment exists for AD today.

Currently, the primary method of diagnosing AD in living patients involves taking detailed patient histories, administering memory and psychological tests, and ruling out other explanations for memory loss, including temporary (e.g., depression or vitamin B12 deficiency) or permanent (e.g., stroke) conditions. These clinical diagnostic methods, however, are not foolproof.

One obstacle to successful treatment is early diagnosis. Typically, clinical diagnostic procedures are only helpful after patients have begun displaying significant, abnormal memory loss or personality changes. By then, a patient has likely had AD for years.

Research has shown that cerebrospinal fluid (“CSF”) samples from AD patients contain higher than normal amounts of tau, which is released as neurons degenerate, and lower than normal amounts of beta amyloid, presumably because it is trapped in the brain in the form of amyloid plaques. Because these biomarkers are released into CSF, a lumbar puncture (or “spinal tap”) is required to obtain a sample for testing. However, these invasive tests are only administered after manifestation of cognitive decline. Methods, for identifying patients at risk of AD and detecting AD at an earlier stage, preferably using less invasive methods than lumber puncture, are needed.

### 3. SUMMARY

In various aspects, the disclosure provides methods for scoring a subject's risk for developing or already having AD and systems configured to generate an AD risk score for a subject. The methods typically comprise generating an AD risk score from a dataset associated with the subject that has quantitative data for at least 4 protein markers in one or more fluid samples from the subject e.g., blood samples such as plasma samples or serum samples, or cerebral spinal fluid (CSF) samples. In preferred embodiments, the samples are blood samples, more preferably plasma samples. As blood samples are easily and commonly obtained as part of routine healthcare screenings, the methods of the disclosure allow for non-invasive identification of subjects at risk of developing AD or having AD, at an early stage. AD risk scores are typically generated using AI-based algorithms. To the inventors' knowledge, the methods of the disclosure are the first non-invasive methods that enable accurate, reliable identification of subjects at risk for AD, even before the display of symptoms associated with AD, using a combination of at least 4 protein markers present in blood. By identifying subjects at risk of developing AD or already having AD at an early stage, immediate treatment (e.g., with an AD therapeutic or candidate AD therapeutic) can initiated. Thus, the methods and systems described herein represent a significant improvement over existing methods and systems in the field of AD prediction, diagnosis, and treatment.

Exemplary protein markers that can be used in the methods of the disclosure include tau peptide markers (e.g., p-tau 181, p-tau 217, p-tau 231, p-tau 235), amyloid peptide markers (e.g., Aβ-40, Aβ-42, the ratio of Aβ-40:Aβ-42, the ratio of Aβ-40:Aβ-42), neurodegeneration markers (e.g., neurofilament light (NFL), glial fibrillary acidic protein (GFAP)), metabolic disorder markers (e.g., HbA1c) and inflammation markers (e.g., soluble TREM 2 (sTREM-2)). The at least 4 protein markers typically include at least 3 of a tau peptide marker, an amyloid peptide marker, a neurodegeneration marker, a metabolic disorder marker, and an inflammation marker. For example, a dataset can include quantitative data for one or more tau peptide markers, one or more amyloid peptide markers, and one or more neurodegeneration markers. In some embodiments, the dataset further includes one or more genetic risk markers of AD (e.g., APO E4) and/or one or more other factors such as age and gender (male or female).

The step of generating an AD risk score is typically computer implemented. A computer implemented method can comprise executing, in a computer system having one or more processors coupled to a memory storing one or more computer readable instructions for execution by the one or more processors, the one or more computer readable instructions comprising instructions for generating an AD risk score for the subject from the dataset. Artificial intelligence (AI) based algorithms for generating AD risk scores can be used. Exemplary AI-based algorithms include those based on logistic regression, light GBM, Random Forest, and CatBoost machine learning models that have been trained using a set of patient records.

An AD risk score (or combination of AD risk scores) can be used to classify a subject into an AD risk category, for example a high, intermediate (sometimes referred to herein as a moderate or medium), or low risk category. The classification can be used to recommend follow-up testing. For example, a recommendation for a subject classified as high risk can be a recommendation for further testing for indicators of AD (e.g., during a neurologist visit), while a recommendation for subjects classified as medium risk or low risk can be a recommendation for re-testing after 1 or more years, e.g., 2-3 years for medium risk and 3-5 years for low risk.

In other aspects, the disclosure provides methods of producing AI-based algorithms for generating an AD risk score. These methods typically comprise executing, in a computer system having one or more processors coupled to a memory storing one or more computer readable instructions for execution by the one or more processors, the one or more computer readable instructions comprising instructions for (a) storing a dataset comprising a plurality of patient records (e.g., at least 100, at least 200, at least 300, at least 500, at least 1000, at least 5000, or more than at least 5000 patient records), each patient record having quantitative data for at least 4 protein markers in one or more fluid samples from the patient and data for one or more AD surrogate variables for the patient and (b) training a machine learning model with at least a portion of the patient records (e.g., at least 100, at least 200, at least 300, at least 500, at least 1000, at least 5000 or more than 5000 patient records), where the quantitative data for the at least 4 protein markers are used as input variables and the data for the one or more AD surrogate variables are used as output variables for training the machine learning model.

An AD surrogate variable is a factor associated with AD risk, for example brain amyloid load, brain tau load, brain neurodegeneration, or clinical diagnosis of mild cognitive impairment (MCI) or AD. Thus, for example, when an AD surrogate value is brain amyloid load, the patient record data can include amyloid PET centiloid data; when an AD surrogate value is brain tau load, the patient record data can include Tau PET SUVR data; when an AD surrogate variable is brain neurodegeneration, the patient record data can include clinical dementia rating data; and when an AD surrogate value is clinical diagnosis of MCI or AD, the patient record data can include data relating to patient diagnosis of MCI or AD. By using the data for the protein markers as input variables and the AD surrogate variables as output variables, the training produces an AI-based algorithm that correlates levels of the protein markers to the AD surrogate variables. Additional input variables, for example, age, gender, education level, genetic risk markers for AD (e.g., APO E4, Clusterin (CLU), Sortilin-related receptor-1 (SORL1), ATP-binding cassette subfamily A member 7 (ABCA7)), age at tau scan, and combinations thereof can also be included.

Exemplary machine learning models that can be used to produce an AI-based algorithm for generating an AD risk score include logistic regression, light GBM, Random Forest, and CatBoost models. Other machine learning models, such as linear discriminant analysis, Adaptive Boosting, Extreme Gradient Boosting, Extra Trees, Naïve-Bayes, K-Nearest neighbor, Gradient Boosting, and Support Vector models, can also be used.

The AI-based algorithms produced by the methods of the disclosure can be used, for example, in the methods for scoring a subject's risk for developing or already having AD described herein.

In further aspects, the disclosure provides systems configured to generate an AD risk score and systems configured to generate an AI-based algorithm for generating an AD risk score. The systems typically include one or more processors coupled to a memory storing one or more computer readable instructions for execution by the one or more processors.

Systems configured to generate an AD risk score can include instructions for generating one or more AD risk scores according to a method for generating an AD risk score as described herein. Such systems can include further instructions for generating a report based on the one or more AD risk scores. The report can include a classification of the subject's risk for developing or having AD. For example, the subject can be classified as having a high risk of having or developing AD, medium (or moderate) risk of developing AD, or low risk of developing AD. The report can further include a recommendation for further testing based on the classification. For example, a recommendation for a subject classified as having a high risk of having or developing AD can be for further testing of the subject for indicators of AD (for example, via a neurologist visit). A recommendation for a subject classified as having a medium risk of developing AD can be a recommendation for re-testing in the future, for example in approximately 1-2 years, e.g. 1 year or 2 years, while a recommendation for a subject classified as having a low risk can be a recommendation for re-testing in the future, for example in approximately 3-5 years, e.g., 3 years, 4 years, or 5 years.

Systems configured to produce an AI-based algorithm for generating an AD risk score can include instructions for generating an AI-based algorithm according to a method for produce an AI-based algorithm as described herein.

In some embodiments, a system configured to produce an AI-based algorithm for generating an AD risk score can further be configured to generate an AD risk score for a subject. For example, the system can have a training mode for producing an AI-based algorithm for generating an AD risk score, and an AD risk score generating mode for generating AD risk scores for subjects.

In further aspects, the disclosure provides tangible, non-transitory computer-readable media comprising instructions generating an AD risk score and/or instructions for producing an AI-based algorithm for generating an AD risk score.

Further exemplary features of the methods of the disclosure are described in Sections 5.2 and 5.5 and specific embodiments 1 to 241 in Section 7.1, infra.

Further exemplary features of protein markers that can be used in the methods of the disclosure are described in Section 5.3 and specific embodiments 104 to 143 in Section 7.1, infra.

Further exemplary features of genetic markers that can be used in the methods of the disclosure are described in Section 5.4 and specific embodiments 91 to 94 in Section 7.1, infra.

Further exemplary features of computer-based methods, systems, and tangible, non-transitory computer-readable media of the disclosure are described in Section 5.5 and specific embodiments 10 to 273 in Section 7.1, infra.

### 4. BRIEF DESCRIPTION OF THE FIGURES

FIGS. 1A-1L show data for the patient records used in Example 3 represented by CL12 amyloid status. FIG. 1A: count of patients having an amyloid PET centiloid value less than 12 (class 0) and 12 or greater (class 1); FIG. 1B: age distribution; FIG. 1C: gender (female=class 0; male=class 1) distribution; FIG. 1D: tau p181 plasma level distribution; FIG. 1E: NFL plasma level distribution; FIG. 1F: Aβ-42 plasma level distribution; FIG. 1G: Aβ-40 plasma level distribution; FIG. 1H: GFAP plasma level distribution; FIG. 1I: sTREM2 plasma level distribution; FIG. 1J: α-Synuclein plasma level distribution; FIG. 1K: TDP43 plasma level distribution; FIG. 1L: adiponectin plasma level distribution.

FIGS. 2A-2L show data for the patient records used in Example 3 represented by CL21 amyloid status. FIG. 2A: count of patients having an amyloid PET centiloid value less than 21 (class 0) and 21 or greater (class 1); FIG. 2B: age distribution; FIG. 2C: gender (female=class 0; male=class 1) distribution; FIG. 2D: tau p181 plasma level distribution; FIG. 2E: NFL plasma level distribution; FIG. 2F: Aβ-42 plasma level distribution; FIG. 2G: Aβ-40 plasma level distribution; FIG. 2H: GFAP plasma level distribution; FIG. 2I: sTREM2 plasma level distribution; FIG. 2J: α-Synuclein plasma level distribution; FIG. 2K: TDP43 plasma level distribution; FIG. 2L: adiponectin plasma level distribution.

FIGS. 3A-3L show data for the patient records used in Example 3 based represented by brain tau load status in the mesial temporal (“MT”) region. FIG. 3A: count of patients having a MK6240 Tau PET SUVR in the mesial temporal region s 1.181 (class 0) and >1.181 (class 1); FIG. 3B: age distribution; FIG. 3C: gender (female=class 0; male=class 1) distribution; FIG. 3D: tau p181 plasma level distribution; FIG. 3E: NFL plasma level distribution; FIG. 3F: Aβ-42 plasma level distribution; FIG. 3G: Aβ-40 plasma level distribution; FIG. 3H: GFAP plasma level distribution; FIG. 3I: sTREM2 plasma level distribution; FIG. 3J: α-Synuclein plasma level distribution; FIG. 3K: TDP43 plasma level distribution; FIG. 3L: adiponectin plasma level distribution.

FIGS. 4A-4L show data for the patient records used in Example 3 represented by brain tau load status in the temporal (“TJ”) region. FIG. 4A: count of patients having a MK6240 Tau PET SUVR in the temporal region s 1.216 (class 0) and >1.2161 (class 1); FIG. 4B: age distribution; FIG. 4C: gender (female=class 0; male=class 1) distribution; FIG. 4D: tau p181 plasma level distribution; FIG. 4E: NFL plasma level distribution; FIG. 4F: Aβ-42 plasma level distribution; FIG. 4G: Aβ-40 plasma level distribution; FIG. 4H: GFAP plasma level distribution; FIG. 4I: sTREM2 plasma level distribution; FIG. 4J: α-Synuclein plasma level distribution; FIG. 4K: TDP43 plasma level distribution; FIG. 4L: adiponectin plasma level distribution.

FIGS. 5A-5L show data for the patient records used in Example 3 represented by clinical dementia rating status. FIG. 5A: count of patients having a clinical dementia rating (CDR) of <0.5 (class 0) and ≥0.5 (class 1); FIG. 5B: age distribution; FIG. 5C: gender (female=class 0; male=class 1) distribution; FIG. 5D: tau p181 plasma level distribution; FIG. 5E: NFL plasma level distribution; FIG. 5F: Aβ-42 plasma level distribution; FIG. 5G: Aβ-40 plasma level distribution; FIG. 5H: GFAP plasma level distribution; FIG. 5I: sTREM2 plasma level distribution; FIG. 5J: α-Synuclein plasma level distribution; FIG. 5K: TDP43 plasma level distribution; FIG. 5L: adiponectin plasma level distribution.

FIGS. 6A-6L show data for the patient records used in Example 3 represented by clinical MCI and AD diagnosis status. FIG. 6A: count of patients having not having a clinical diagnosis of MCI or AD (class 0) and having a clinical diagnosis of MCI or AD (class 1); FIG. 6B: age distribution; FIG. 6C: gender (female=class 0; male=class 1) distribution; FIG. 6D: tau p181 plasma level distribution; FIG. 6E: NFL plasma level distribution; FIG. 6F: Aβ-42 plasma level distribution; FIG. 6G: Aβ-40 plasma level distribution; FIG. 6H: GFAP plasma level distribution; FIG. 6I: sTREM2 plasma level distribution; FIG. 6J: α-Synuclein plasma level distribution; FIG. 6K: TDP43 plasma level distribution; FIG. 6L: adiponectin plasma level distribution.

FIGS. 7A-7F show ROC curves of artificial intelligence-based algorithms for predicting whether a subject is likely to have an amyloid PET centiloid value less than 12 (CL12) (Example 3). FIG. 7A: CatBoost-based algorithm; FIG. 7B: Random Forest (RF)-based algorithm; FIG. 7C: Logistic Regression (LR)-based algorithm; FIG. 7D: Light GBM-based algorithm; FIG. 7E: Linear Discriminant Analysis (LDA)-based algorithm; FIG. 7F: Blender (from blending of CatBoost, RF, LR, Light GBM, and LDA-based algorithms).

FIGS. 8A-8F show ROC curves of artificial intelligence-based algorithms for predicting whether a subject is likely to have an amyloid PET centiloid value greater than or equal to 21 (CL21) (Example 3). FIG. 8A: CatBoost-based algorithm; FIG. 8B: Random Forest (RF)-based algorithm; FIG. 8C: Logistic Regression (LR)-based algorithm; FIG. 8D: Light GBM-based algorithm; FIG. 8E: Linear Discriminant Analysis (LDA)-based algorithm; FIG. 8F: Blender (from blending of CatBoost, RF, LR, Light GBM, and LDA-based algorithms).

FIGS. 9A-9F show ROC curves of artificial intelligence-based algorithms for predicting whether a subject is likely to have a brain tau load (MT) above a cutoff value (Example 3). FIG. 9A: CatBoost-based algorithm; FIG. 9B: Random Forest (RF)-based algorithm; FIG. 9C: Logistic Regression (LR)-based algorithm; FIG. 9D: Light GBM-based algorithm; FIG. 9E: Linear Discriminant Analysis (LDA)-based algorithm; FIG. 9F: Blender (from blending of CatBoost, RF, LR, Light GBM, and LDA-based algorithms).

FIGS. 10A-10F show ROC curves of artificial intelligence-based algorithms for predicting whether a subject is likely to have a brain tau load (TJ) above a cutoff value (Example 3). FIG. 10A: CatBoost-based algorithm; FIG. 10B: Random Forest (RF)-based algorithm; FIG. 10C: Logistic Regression (LR)-based algorithm; FIG. 10D: Light GBM-based algorithm; FIG. 10E: Linear Discriminant Analysis (LDA)-based algorithm; FIG. 10F: Blender (from blending of CatBoost, RF, LR, Light GBM, and LDA-based algorithms).

FIGS. 11A-11F show ROC curves of artificial intelligence-based algorithms for predicting whether a subject is likely to have a clinical dementia rating (CDR) greater than or equal to 0.5 (Example 3). FIG. 11A: CatBoost-based algorithm; FIG. 11B: Random Forest (RF)-based algorithm; FIG. 11C: Logistic Regression (LR)-based algorithm; FIG. 11D: Light GBM-based algorithm; FIG. 11E: Linear Discriminant Analysis (LDA)-based algorithm; FIG. 11F: Blender (from blending of CatBoost, RF, LR, Light GBM, and LDA-based algorithms).

FIGS. 12A-12F show ROC curves of artificial intelligence-based algorithms for predicting whether a subject is likely to have symptoms sufficient for a diagnosis of MCI or AD (Example 3). FIG. 12A: CatBoost-based algorithm; FIG. 12E: Random Forest (RF)-based algorithm; FIG. 12C: Logistic Regression (LR)-based algorithm; FIG. 12D: Light GBM-based algorithm; FIG. 12E: Linear Discriminant Analysis (LDA)-based algorithm; FIG. 12F: Blender (from blending of CatBoost, RF, LR, Light GBM, and LDA-based algorithms).

FIG. 13 shows a feature importance plot for a Random Forest-based algorithm for predicting whether a subject is likely to have an amyloid PET centiloid value greater less than 12 (CL12) (Example 3).

FIG. 14 shows a feature importance plot for a CatBoost-based algorithm for predicting whether a subject is likely to have an amyloid PET centiloid value greater than or equal to 21 (CL21) (Example 3).

FIG. 15 shows a feature importance plot for a CatBoost-based algorithm for predicting whether a subject is likely to have a brain tau load (MT) above a cutoff value (Example 3).

FIG. 16 shows a feature importance plot for a CatBoost-based algorithm for predicting whether a subject is likely to have a brain tau load (TJ) above a cutoff value (Example 3).

FIG. 17 shows a feature importance plot for a Light GBM-based algorithm for predicting whether a subject is likely to have a clinical dementia rating (CDR) greater than or equal to 0.5 (Example 3).

FIG. 18 shows a feature importance plot for a Logistic Regression-based algorithm for predicting whether a subject is likely to have symptoms sufficient for a diagnosis of MCI or AD (Example 3).

FIG. 19 shows an exemplary flowchart for classifying a subject as high, medium, or low risk for AD based on AD risk scores for surrogate markers of AD (Example 4).

FIG. 20 shows an exemplary PREFER-AD report (Example 4).

### 5. DETAILED DESCRIPTION

The present invention addresses the need in the art for systems for and methods of early and/or non-invasive detection of AD and/or non-invasive assessment of the risk of developing AD.

### 5.1. Definitions

Unless defined otherwise herein, all technical and scientific terms used herein have the same meaning as commonly understood by one of ordinary skill in the art. Various scientific dictionaries that include the terms included herein are well known and available to those in the art.

As used herein the term “protein” refers to a polymer of amino acid residues and the term “peptide” refers to a short protein or a segment of a protein, e.g. a protein or segment of 100 or fewer amino acids.

As used herein, the singular forms “a”, “an” and “the” include plural referents unless the content and context clearly dictates otherwise. Thus, for example, reference to “a protein marker” includes a combination of two protein markers, a combination of three protein markers, and the like.

Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

The terms “about”, “approximately” and the like is used throughout the specification in front of a number to show that the number is not necessarily exact (e.g., to account for fractions of the time periods recited (e.g., 360 days is approximately one year and one year and 11 months is approximately two years, etc.), variations in measurement accuracy and/or precision, timing, etc.). It should be understood that a disclosure of “about X” or “approximately X” where X is a number is also a disclosure of “X.” Thus, for example, a disclosure of an embodiment in which AD risk scoring is repeated after “about 2 years” is also a disclosure of an embodiment in the AD risk scoring is repeated after “2 years.”

The term “AD therapeutic” refers to an agent or combination of agents (e.g., small molecule drugs or biologics such as antibodies) useful for treating or ameliorating (e.g., slowing, reversing or abating) signs, symptoms or underlying etiology of AD. A “candidate AD therapeutic” is an agent or combination of agents (e.g., small molecule drugs or biologics such as antibodies) believed to be useful for treating or ameliorating (e.g., slowing, reversing or abating) signs, symptoms or underlying etiology of AD which does not yet have regulatory approval. Candidate AD therapeutics include agents and combinations of agents in clinical trials.

The term “artificial intelligence-based algorithm” refers to an algorithm that has been produced using artificial intelligence (AI), for example using one or more machine learning models. For example, an artificial intelligence-based algorithm can be an algorithm resulting from training a machine learning model using a plurality of patient records. Examples of machine learning models that can be used to produce an AI-based algorithm include, but are not limited to logistic regression, light GBM, Random Forest, CatBoost, linear discriminant analysis, Adaptive Boosting, Extreme Gradient Boosting, Extra Trees, Naïve-Bayes, K-Nearest neighbor, Gradient Boosting, and Support Vector models. Blending can be used to combine predictions from two or more base models, e.g., two or more types of the foregoing models.

### 5.2. Methods of the Disclosure

In certain aspects, the disclosure provides a method for scoring a subject's risk of developing or already having AD. Scoring the subject's AD risk score can comprise (a) determining the levels of at least 4 protein markers in one or more fluid samples from the subject and (b) combining the levels of at least 4 protein markers to generate an AD risk score for the subject, thereby scoring the subject's risk of developing or already having AD. In some embodiments, scoring the subject's AD risk score can comprise (a) determining the levels of at least 4 or at least 5 protein markers in one or more fluid samples from the subject and (b) combining the levels of at least 4 or at least 5 protein markers to generate an AD risk score for the subject, thereby scoring the subject's risk of developing or already having AD.

Brain amyloid load, brain tau load, brain neurodegeneration, and exhibition of symptoms sufficient for a diagnosis of mild cognitive impairment or AD are linked to AD risk. Thus, an AD risk score that predicts a subject's brain amyloid load, brain tau load, brain neurodegeneration, or whether the subject exhibits symptoms sufficient for a diagnosis of mild cognitive impairment or AD can be used as a surrogate to assess a subject's risk of developing or already having AD. A single AD risk score that predicts a subject's brain amyloid load, brain tau load, brain neurodegeneration, or whether the subject exhibits symptoms sufficient for a diagnosis of mild cognitive impairment or AD can be generated for a subject to score the subject's risk of developing or already having AD or, alternatively, multiple AD risk scores can be generated.

For example, a dataset comprising quantitative data for at least 4 protein markers can be used to generate two or more, three or more, four or more, five or more, or six or more individual AD risk scores that individually predict (i) the subject's brain amyloid load, (ii) the subject's brain tau load, (iii) brain neurodegeneration in the subject, or (iv) whether the subject exhibits symptoms sufficient for a diagnosis of mild cognitive impairment or AD.

In further aspects, the disclosure provides a method of analyzing a sample from a subject comprising the steps of (a) obtaining one or more fluid samples from a subject; (b) performing an antibody or antigen assay on the one or more fluid samples to measure the levels of at least 4 or at least 5 protein markers; (c) generating quantitative values of the at least 4 or at least 5 protein markers; (d) storing the quantitative values in a dataset associated with the subject; and (e) scoring the sample with an initial AD risk score comprising the levels of the at least 4 or at least 5 protein markers, thereby analyzing the sample from the subject.

The fluid samples are typically selected from blood, serum and cerebral spinal fluid (CSF). In some embodiments, the samples are blood samples. In some embodiments, the samples comprise a combination of blood and CSF samples. Preferably, the fluid samples comprise or consist of blood samples (e.g., whole blood samples, plasma samples or serum samples). Preferably, the risk score utilizes at least 3, at least 4 or at least 5 protein markers in blood and/or serum, and in some embodiments utilize 6, 7, 8, 9 or 10 protein markers in blood and/or serum. In some embodiments, the risk score utilizes at least 3, at least 4 or at least 5 protein markers in blood and/or serum, and in some embodiments utilize 6, 7, 8, 9 or 10 protein markers in blood samples which are plasma samples.

Typically, the protein markers comprise at least 4 of a tau peptide marker (e.g., as described in Section 5.3.2), an amyloid peptide marker (e.g., as described in Section 5.3.1), a neurodegeneration marker (e.g., as described in Section 5.3.3), a metabolic disorder marker (which can be a diabetes marker) (e.g., as described in Section 5.3.4), and an inflammation marker (e.g., as described in Section 5.3.5).

The protein markers are preferably detected in blood and/or serum and can be used in combination with one or more protein markers in CSF (including but not limited to one or more of tau, amyloid, and/or neurodegeneration markers) and/or one or more genetic markers, e.g., APO E4, Clusterin (CLU), Sortilin-related receptor-1 (SORL1), or ATP-binding cassette subfamily A member 7 (ABCA7), most preferably APO E4. In some embodiments, APO E4 status is not used. Other factors that can be incorporated into the risk score include the subject's family history of AD, the age, gender (male or female) and education of the subject (including combinations thereof), and the subject's performance on a cognitive assessment such as the Alzheimer's Initiative Preclinical Composite Cognitive test (“APCC”) (see, e.g., Langbaum et al., 2020, Alzheimer's Research & Therapy 12:66).

Optionally, the methods can comprise measuring the levels of the markers used in calculating the risk score, for example using antibody assays and/or antigen assays for protein markers and PCR or sequencing assays for nucleic acid markers, which in turn can optionally be preceded by obtaining the relevant fluid sample(s). Exemplary methods of detecting the protein markers are described in Section 5.3 and exemplary methods of detecting genetic markers are described in Section 5.4.

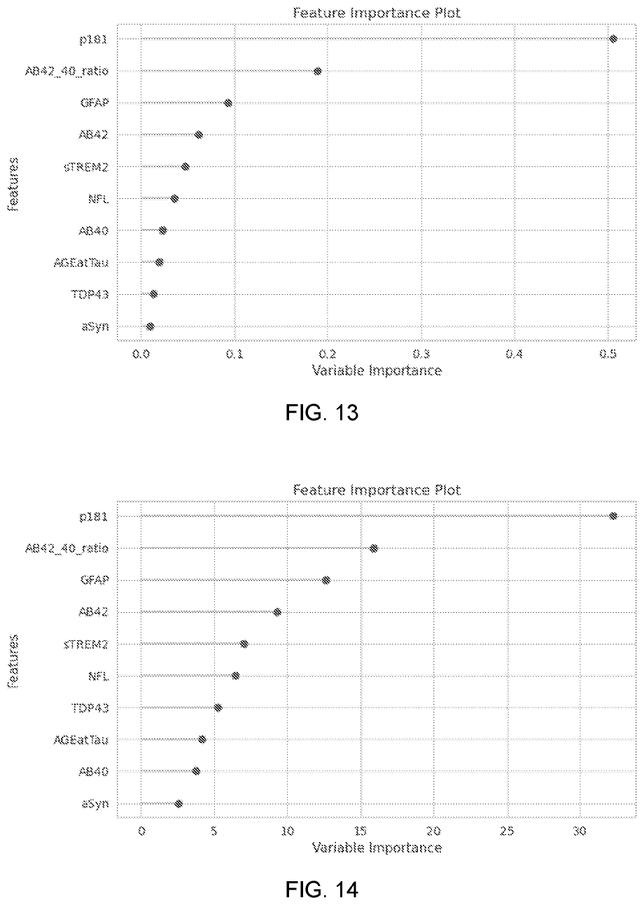

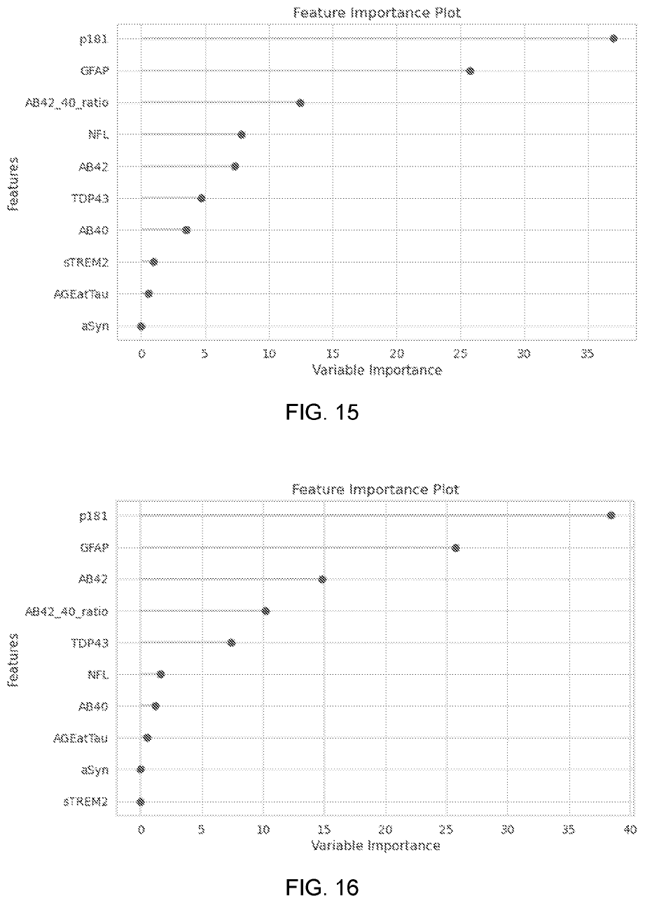

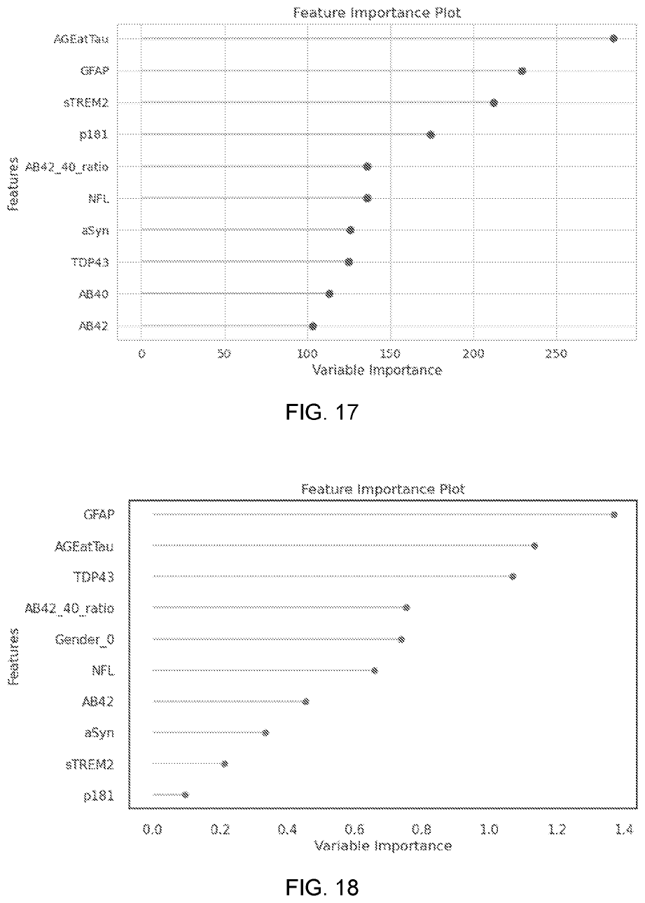

The levels of the protein markers utilized in the risk score and optionally additional factors (e.g., the subject's genetic marker status, the subject's family history of AD, the subject's date of birth or age, the subject's gender, the subject's education, the subject's performance on a cognitive assessment, the subject's prior risk scores, or any combination thereof) can be input into a dataset associated with the subject, which can then be utilized to perform the risk score calculation. The risk score calculation can be performed using a statistics- and/or artificial intelligence-based algorithm. In various embodiments, the levels of some (e.g., at least 2 or at least 3) of the protein markers are weighted equally and/or the levels of some (at least 2 or at least 3) of the protein markers are weighted differentially (e.g., a first peptide marker can be said to be weighted greater than a second peptide marker when the first peptide marker has a higher variable importance than the second peptide marker in a feature importance plot for an artificial intelligence-based algorithm, for example as show in FIGS. 13-18). In a particular embodiment, the levels of all the protein markers are weighted equally. In another particular embodiment, the levels of all the protein markers are weighted differentially. The risk score can be presented in the form of a percentage, multiplier value or absolute score. The risk score can also be presented as a prediction, for example a binary prediction that the subject is positive or negative for a risk factor for AD.

Alternatively, or in addition, a risk score can be binned into an AD risk category, for example high, moderate (or medium) or low. The risk category can then be presented, for example in a report. When multiple AD risk scores are generated, each AD risk score can be independently binned.

Calculation and the optional binning of a subject's risk score(s) can be performed by a computer. Accordingly, the present disclosure provides a computer implemented method for assessing a subject's risk for developing or already having AD, comprising, in a computer system having one or more processors coupled to a memory storing one or more computer readable instructions for execution by the one or more processors, the one or more computer readable instructions comprising instructions for (a) storing a dataset comprising a plurality of patient records, each patient record comprising quantitative data for at least 4 or at least 5 protein markers in one or more fluid samples from the subject and (b) generating an AD risk score for the subject using a weighted scoring system for the at least 4 or at least 5 protein markers, e.g., using a statistics- and/or artificial intelligence-based algorithm. The dataset can be stored on a local server or on a remote server, e.g., in the cloud.

The disclosure further provides a computer implemented method for assessing a subject's risk for developing or already having AD, the method comprising executing, in a computer system having one or more processors coupled to a memory storing one or more computer readable instructions for execution by the one or more processors, the one or more computer readable instructions comprising instructions for (a) storing a dataset associated with the subject, wherein said dataset comprises quantitative data for at least 4 protein markers in one or more fluid samples from the subject and (b) generating an AD risk score for the subject from the dataset, e.g., using an artificial intelligence-based algorithm. The dataset can be stored on a local server or on a remote server, e.g., in the cloud.

The risk score(s) generated according to a method of the disclosure can be provided, for example, as a percentage, multiplier value absolute score, or prediction (e.g., positive or negative) and the computer may further perform binning of the score as described herein. A notification may be provided to the user recommending further testing when the subject's risk score is indicative of a high risk for developing AD. A notification may be provided to the user recommending re-testing in the future when the subject's risk score(s) is/are indicative of a moderate or low risk for developing AD.

The risk score of a particular subject can be binned into at least two categories, reflecting the level of risk (e.g., the likelihood) of having or developing AD. Preferably, the risk score is binned into three categories, reflecting a low risk of developing AD, an intermediate risk of developing AD (also referred to herein as moderate or medium risk), and a high risk of developing AD (which may include subjects who have already developed AD, although the risk score of subjects who have already developed AD may be binned into a separate category).

When multiple risk scores are generated for a subject (e.g., two or more AD risk scores that individually predict (i) the subject's brain amyloid load, (ii) the subject's brain tau load, (iii) brain neurodegeneration in the subject, or (iv) whether the subject exhibits symptoms sufficient for a diagnosis of mild cognitive impairment or AD) each risk score can be individually binned as described herein. A notification may be provided to the user recommending further testing (e.g., via a neurologist visit) when any one of the subject's risk scores is indicative of a high risk for developing AD. A notification may be provided to the user recommending re-testing in the future when the subject's risk score(s) is/are indicative of a moderate or low risk for developing AD.

When one or more of a subject's risk scores are indicative of a high risk for having or developing AD, the subject can be classified as having a high risk of having or developing AD. When none of the subject's AD risk scores are indicative of a high risk for AD, but one or more of the subject's AD risk scores are indicative of a moderate (or medium) risk of developing AD, the subject can be classified as having a moderate (or medium) risk of developing AD. When none of the subject's AD risk scores are indicative of a high risk for AD and none of the subject's AD risk scores are indicative of a moderate (or medium) risk of AD, the subject can be classified as having a low risk of developing AD.

The present disclosure provides methods for monitoring the health of individuals by repeatedly scoring their risk of developing AD over several years. Screening of subject's AD risk by performing the methods described herein can begin at an early age, particularly subjects with known risk factors such as APO E4 status or familial history of AD. In various embodiments, the subject whose risk score is determined is 30-39 years of age, 40-49 years of age, 50-59 years of age, 60-69 years of age or 70-79 years of age. The risk score determination can be repeated after a year or more than a year, for example on an annual, biennial, triennial, quadrennial or quinquennial basis.

The frequency of scoring a subject's risk of AD might be informed by the most recent risk score or any changes to the risk score, with subjects at lower risk scores being evaluated at lesser frequency than subjects with higher risk scores or recent increases in risk score. For example, if a subject's risk score(s) is/are binned into a low risk category (and/or if a subject is classified as having a low risk of AD), their risk score may be reassessed in approximately 3-5 years, whereas a subject whose risk score(s) is/are binned into an intermediate category (and/or who is classified as having an intermediate risk of AD) may have their risk score(s) reassessed in approximately 1-2 years. If a subject has a high risk score, a marked increase in their risk score, or a risk score indicative of already having developed AD, then further testing might be warranted, for example PET amyloid and/or tau scans, amyloid scanning methods, lumbar puncture amyloid and/or tau procedures, structural MRI, neuropsychological testing and/or a combination thereof. The neuropsychological testing may comprise one or more memory and/or cognitive tests, for example the APCC. If it is determined that the subject has already developed AD, one or more approved AD therapeutics (e.g., aducanumab-avwa) and/or candidate AD therapeutics (e.g., a candidate AD therapeutic that is the subject of a clinical trial) can be administered to the subject, for example an amyloid disease modifying therapy, a tau therapy, a cholinesterase inhibitor, an NMDA receptor blocker, or a combination thereof. Accordingly, the present methods can be useful for identifying and enrolling patients into clinical trials of candidate AD therapeutics.

The risk score algorithm can be developed by analyzing protein marker levels (at least 3 or at least 4 in blood and/or serum and optionally one or more in CSF) from a plurality of subjects with AD and a plurality of cognitively normal individuals, optionally also from individuals with mild cognitive impairment (MCI) and/or one or more dementias other than AD (e.g., Lewy Body dementia or frontal lobe dementia). The analysis can include other factors, e.g., one or more of the genetic markers disclosed herein, the subject's family history of AD, the age/date of birth, gender and education of the subject, and the subject's performance on a cognitive assessment such as the Alzheimer's Initiative Preclinical Composite Cognitive test (“APCC”). Multivariate analysis, e.g., using statistical and/or artificial intelligence approaches, are applied to determine the correlation between combinations of protein markers and other factors on subjects' likelihood to develop AD in order to identify the top 5, 6, 7, 8, 9 or 10 components to utilize in the risk score algorithm. The analysis can be performed on datasets from at least 100, more preferably at least 200, and most preferably at least 300 individuals. In an exemplary embodiment, 50-60% of the individuals are cognitively normal, 15-25% of individuals are diagnosed with MCI, 10-20% of the individuals are diagnosed with AD, and 5% of individuals are diagnosed a dementia other than AD.

In some aspects of the disclosure, an AD risk score algorithm is developed using a machine learning model, for example a logistic regression model, a light GBM model, a Random Forest model, or a CatBoost model. Other machine learning models can also be used, for example linear discriminant analysis, Adaptive Boosting, Extreme Gradient Boosting, Extra Trees, Naïve-Bayes, K-Nearest neighbor, Gradient Boosting, and Support Vector models.

Protein marker levels can be used as input variables and one or more AD surrogate variables can be used as the output variables to determine the correlation between the protein marker levels and the one or more AD surrogate variables. Additional input variables, for example, age, gender, education level, genetic risk markers for AD (e.g., APO E4, Clusterin (CLU), Sortilin-related receptor-1 (SORL1), ATP-binding cassette subfamily A member 7 (ABCA7)), age at tau scan, and combinations thereof can also be included. AD surrogate variables are factors linked to AD risk, for example brain amyloid load, brain tau load, brain neurodegeneration, or clinical diagnosis of mild cognitive impairment (MCI) or AD. Thus, the machine learning model can be used to generate an algorithm that correlates the input variables with the one or more AD surrogate variables.

Methods for producing an AD risk score algorithm can comprise executing, in a computer system having one or more processors coupled to a memory storing one or more computer readable instructions for execution by the one or more processors, the one or more computer readable instructions comprising instructions for (a) storing a dataset comprising a plurality of patient records, each patient record comprising quantitative data for at least 4 protein markers in one or more fluid samples from the patient and data for one or more AD surrogate variables for the patient and (b) training a machine learning model with at least a portion of the patient records, where the quantitative data for the at least 4 protein markers are input variables and the data for the one or more AD surrogate variables are output variables for the machine learning model. An AI-based algorithm for generating an AD risk score produced by such methods can be used in the methods for scoring a subject's risk for developing or already having AD described herein.

### 5.3. Protein Markers

Generally, the protein markers comprise at least 3 of a tau peptide marker, an amyloid peptide marker, a neurodegeneration marker, a metabolic disorder marker (which can be a diabetes marker), and an inflammation marker. Typically, the protein markers comprise at least 4 of a tau peptide marker, an amyloid peptide marker, a neurodegeneration marker, a metabolic disorder marker (which can be a diabetes marker), and an inflammation marker. The term “peptide” refers to a short protein or a segment of a protein.

5.3.1. Amyloid Markers

Examples of amyloid peptide markers that can be used in determining the risk score are Aβ40, Aβ42, and the ratio between them (e.g., Aβ42:Aβ40 or Aβ40:Aβ42). Aβ40 is a 40 amino acid proteolytic product from the amyloid precursor protein (APP) that has gained attention as a biomarker correlating with Alzheimer disease (AD) onset, mild cognitive impairment, vascular dementia, and other cognitive disorders.

Beta-secretase cleavage of APP initially results in the production of an APP fragment that is further cleaved by gamma-secretase at residues 40-42 to generate two main forms of amyloid beta, Aβ40 and Aβ42. Amyloid beta (Aβ) peptides (including a shorter Aβ38 isoform) are produced by different cell types in the body, but the expression is particularly high in the brain. Accumulation of Aβ in the form of extracellular plaques is a neuropathological hallmark of AD and believed to play a central role in the neurodegenerative process. Aβ40 is the major amyloid component in these plaques and is thought to be an initiating factor of AD plaques.

In healthy and disease states Aβ40 is the most abundant form of the amyloid peptides in both cerebrospinal fluid (CSF) and plasma (10-20× higher than Aβ42). An inverse relationship exists between Aβ-42 levels in the brain and in the CSF: when Aβ-42 accumulates in amyloid plaques, less of it leaves the brain to enter the CSF and thus CSF Aβ-42 measurements in AD patients are generally lower than for healthy patients.

A combination of Aβ-42 and t-tau (total tau) in CSF can discriminate between patients with stable MCI and patients with progressive MCI into AD or other types of dementia with a sufficient sensitivity and specificity (Frankfort et al., 2008, Current clinical pharmacology, 3(2), 123-131). Regression analyses have shown that pathological CSF (with decreased Aβ-42 and increased tau levels) is a very strong predictor for the progression of MCI into AD.

An inverse association has been found between Aβ42/40 plasma ratios and fibrillary Aβ deposition as amyloid plaques in the brain as measured by amyloid PET scans (Fandos et al, 2017, Alzheimers Dement. 8, 179-187; Ovod et al, 2017, Alzheimers Dement. 13(8):841-849; Nakamura et al., 2018, Nature, 554(7691):249-254; Schindler et al., 2019, Neurology. 93(17): e1647-e1659).

Methods for detecting amyloid-β (Aβ) 42 and Aβ 40 disclosed in, e.g., WO2007140843A2, WO2011033046A1 and U.S. Pat. No. 8,425,905B2 can be utilized in the methods disclosed herein. Commercially available reagents can also be used, e.g., the Simoa™ Aβ40 and Simoa™ Aβ42 Advantage Kits from Quanterix.

5.3.2. Tau Markers

Examples of tau peptide markers that can be used in determining the risk score are phosphorylated tau peptides, e.g., p-tau 181 and p-tau 217. Additional phosphorylated tau peptide markers that can be used include p-tau 231 and p-tau 235. Tau is found in neurofibrillary tangles, which are insoluble twisted fibers found inside the brain's cells. These tangles consist primarily of tau protein, which forms part of microtubules that transport nutrients and other substances from one part of the nerve cell to another. In Alzheimer's disease (AD), however, the tau protein is abnormal and the microtubule structures collapse. Tau accumulation continues throughout the course of the disease. Beginning in the entorhinal cortex and hippocampus, tau continues to accumulate as AD progresses. The total amount of abnormal tau in the AD brain is linked to disease stage and severity. Tangles form when tau is misfolded to form a C-shape in the core of the tangle with a loose end sticking out randomly. Once a tangle has been started, more tau proteins are recruited to make it longer. Tangles form inside of neurons and interfere with the cellular machinery used to create and recycle proteins, which ultimately kills the cell. Several studies have demonstrated that plasma p-tau phosphorylated at threonine 181 (p-tau-181) increases in AD at mild cognitive impairment (MCI) and moderate stages (Tatebe et al., 2017, Mol. Neurodegener. 12:63; Mielke et al., 2018, Alzheimers Dement. 14:989-997). Levels of p-tau-181 in the blood can differentiate AD patients from other tauopathies at symptomatic stages of AD with accuracy (Janelidze et al., 2020, Nat. Med. 26:379-386; Thijssen et al., 2020, Nat. Med. 26:387-397).

Cerebral spinal fluid (CSF) tau phosphorylation levels on threonine 217 (p-tau-217) are closely associated with amyloidosis, improving identification of amyloidosis at the asymptomatic stage (Barthélemy et al., 2015, Alzheimers Dement. 11(7S_Part_19):870). CSF hyperphosphorylation of p-tau-T217 is more accurate than other sites, such as T181 (Barthélemy et al., 2020, J Exp Med 217 (11): e20200861; Barthélemy et al., 2020, Alzheimers Res. Ther. 12:26; Janelidze et al., 2020, Nat. Commun. 11:1683) and T205 (Barthélemy et al., 2020, Nat. Med. 26:398-407), to detect the presence of amyloid plaques. Changes in plasma p-tau-217 highly mirror specific modifications in CSF to detect phosphorylation changes in soluble tau and amyloidosis.

Tau-specific phospho-antibodies useful for detecting p-tau-181 and p-tau-217 are disclosed in, e.g., WO2019213612, EP2764022B1 and US20180282401A1 can be utilized in the methods disclosed herein. Commercially available reagents can also be used, e.g., the Simoa® pTau-181 Advantage Kit, Simoa® pTau-181 Advantage V2 Kit, or Simoa® pTau-231 Advantage Kit from Quanterix, the Tau (Phospho-Thr217) Antibody from SAB (Signalway Antibody), and the pTau-235 antibody RN235 (Sigma-Aldrich).

5.3.3. Neurodegeneration Markers

Examples of neurodegeneration markers that can be used in determining the risk score are neurofilament light (“NFL”) and glial fibrillary acidic protein (“GFAP”).

5.3.3.1. Neurofilament Light

Neurofilament light (NFL) is a 68 kDa cytoskeletal intermediate filament protein that is expressed in neurons. It associates with the 125 kDa Neurofilament medium (NFM) and the 200 kDa Neurofilament heavy (NFH) to form neurofilaments. These molecules are major components of the neuronal cytoskeleton and are believed to function primarily to provide structural support for the axon and to regulate axon diameter. Neurofilaments can be released in significant quantity following axonal damage or neuronal degeneration. NFL has been shown to associate with traumatic brain injury, multiple sclerosis, frontotemporal dementia and other neurodegenerative diseases and can be detected in the blood (Bacioglu et al., 2016, Neuron, 91(1):56-66) similarly to CSF (Preische et al., 2019, Nature medicine, 25(2):277-283). NFL changes are detected around the time of AD symptom onset, over a decade after abnormal AD amyloidosis (Bateman et al., 2012, N. Engl. J. Med. 367:795-804). Plasma NFL levels increase in response to amyloid-related neuronal injury in preclinical stages of Alzheimer's disease, and is related to tau-mediated neurodegeneration in symptomatic patients.

Methods for detecting NFL disclosed in, e.g., US20150268252A1 and EP3129780A1 can be utilized in the methods disclosed herein. Commercially available reagents can also be used, e.g., the Simoa® NF-Light Advantage Kit from Quanterix and Human NEFL ELISA Kit from Elabscience.

5.3.3.2. GFAP

Glial fibrillary acidic protein (GFAP) is a type III intermediate filament (IF) protein that is expressed by numerous cell types of the central nervous system (CNS).

GFAP is closely related to the other three non-epithelial type III IF family members, vimentin, desmin and peripherin, which are all involved in the structure and function of the cell's cytoskeleton. GFAP is thought to help to maintain astrocyte mechanical strength (Cullen et al., 2007, Brain Research. 1158:103-15) as well as the shape of cells, but its exact function remains poorly understood, despite the number of studies using it as a cell marker. The protein was named and first isolated and characterized in 1969 (Eng et al., 2000, Neurochemical Research. 25 (9-10): 1439-51). In AD, GFAP levels are increased in serum and correlates with cognitive impairment (Oeckl et al., 2019, Journal of Alzheimer's disease, 67(2):481-488).

Methods for detecting GFAP are disclosed in, e.g., US20060240480A1, WO2011160096A3, WO2018067474A1 and WO2010019553A2 and can be utilized in the methods disclosed herein. Commercially available reagents can also be used, e.g., the Biovendor Glial Fibrillary Acidic Protein Human ELISA (GFAP) and Invitrogen GFAP Human ProcartaPlex™ Simplex Kits.

5.3.4. Metabolic Disorder Markers

Examples of metabolic disorder markers, e.g., diabetes markers, that can be used in determining the risk score include HbA1c and adiponectin. Glycated hemoglobin (HbA1c) is a form of hemoglobin (Hb) that is chemically linked to a sugar. Most monosaccharides, including glucose, galactose and fructose, spontaneously (i.e., non-enzymatically) bond with hemoglobin, when present in the bloodstream of humans. The formation of the sugar-hemoglobin linkage indicates the presence of excessive sugar in the bloodstream, often indicative of diabetes. The process by which sugars attach to hemoglobin is called glycation. HbA1c is a measure of the beta-N−1-deoxy fructosyl component of hemoglobin. Adiponectin is involved in regulating glucose levels. High adiponectin levels correlate with a lower risk of type 2 diabetes.

Epidemiological studies indicate that diabetes significantly increases the risk of developing AD, suggesting that diabetes may play a causative role in the development of AD pathogenesis. Therefore, elucidating the molecular interactions between diabetes and AD is of critical significance because it might offer a novel approach to identifying mechanisms that may modulate the onset and progression of sporadic AD cases (Baglietto-Vargas et al., 2016, Neuroscience and biobehavioral reviews, 64:72-287).

Methods for detecting HBA1c are disclosed in, e.g., EP1624307A3 and US20120305395A1, and can be utilized in the methods disclosed herein. Commercially available reagents can also be used, e.g., GHbA1c ELISA Kit from Biomatik.

Methods for detecting adiponectin are disclosed in, e.g., U.S. Pat. No. 8,026,345, and can be utilized in the methods disclosed herein. Commercially available reagents can also be used, e.g., Adiponectin Human ELISA Kit from Invitrogen.

5.3.5. Inflammation Markers

Examples of inflammation markers that can be used in determining the risk score are C reactive protein (“CRP”), interleukin-6 (“IL-6”), tumor necrosis factor (“TNF”), soluble TREM 2 (“sTREM-2”), heat shock proteins, and YKL-40.

5.3.5.1. CRP

C-reactive protein (CRP) is an annular (ring-shaped), pentameric protein found in blood plasma, whose circulating concentrations rise in response to inflammation. It is an acute-phase protein of hepatic origin that increases following interleukin-6 secretion by macrophages and T cells. Its physiological role is to bind to lysophosphatidylcholine expressed on the surface of dead or dying cells (and some types of bacteria) in order to activate the complement system via C1q (Thompson D et al., 1999, Structure 7 (2): 169-77). CRP is synthesized by the liver in response to factors released by macrophages and fat cells (adipocytes). It is a member of the pentraxin family of proteins. C-reactive protein was the first pattern recognition receptor (PRR) to be identified (Mantovani et al., 2008, Journal of Clinical Immunology 28 (1): 1-13). Midlife elevations in CRP levels are associated with increased risk of AD development, though elevated CRP levels are not useful for prediction in the immediate prodrome years before AD becomes clinically manifest. However, for a subgroup of patients with AD, elevated CRP predicted increased dementia severity suggestive of a possible proinflammatory endophenotype in AD (O'Bryant et al., 2010, Journal of geriatric psychiatry and neurology, 23(1), 49-53).

Methods for detecting CRP are disclosed in, e.g., US20060246522A1 and can be utilized in the methods disclosed herein. Commercially available reagents can also be used, e.g., ELISA kits available from R&D Biosystems or Sigma Aldrich.

5.3.5.2. IL6

Interleukin 6 (IL-6 or IL6) is an interleukin that acts as both a pro-inflammatory cytokine and an anti-inflammatory myokine. In humans, it is encoded by the IL6 gene (Ferguson-Smith et al., 1988, Genomics. 2 (3): 203-8).

IL-6's role as an anti-inflammatory myokine is mediated through its inhibitory effects on TNFα and IL-1, and activation of IL-1ra and IL-10. There is a growing body of evidence which supports the hypothesis of faulty immune regulation and autoimmunity or inflammatory processes as viable mechanisms of the pathogenesis of Alzheimer's disease. Low levels of IL-6 are present in brain under physiological conditions. A dramatic increase in expression and secretion of IL-6 is observed during various neurological disorders including AD (Benveniste, 1998, Cytokine Growth Factor Rev., 9:259-275).

Methods for detecting IL6 are disclosed in, e.g., WO2011116872A1, U.S. Pat. No. 7,919,095B2 and U.S. Pat. No. 5,965,379A and can be utilized in the methods disclosed herein. Commercially available reagents can also be used, e.g., the Proteintech: AuthentiKine™ Human IL-6 ELISA Kit and the Sigma Aldrich Human IL-6 ELISA Kit.

5.3.5.3. TNF

Tumor necrosis factor (TNF) is a cell signaling protein (cytokine) involved in systemic inflammation and is one of the cytokines that make up the acute phase reaction. It is produced chiefly by activated macrophages, although it can be produced by many other cell types such as T helper cells, natural killer cells, neutrophils, mast cells, eosinophils, and neurons. TNF is a member of the TNF superfamily, consisting of various transmembrane proteins with a homologous TNF domain.

The primary role of TNF is in the regulation of immune cells. TNF, being an endogenous pyrogen, is able to induce fever, apoptotic cell death, cachexia, inflammation and to inhibit tumorigenesis, viral replication, and respond to sepsis via IL-1 and IL-6-producing cells. Dysregulation of TNF production has been implicated in a variety of human diseases including Alzheimer's disease (Swardfager et al., 2010, Biol Psychiatry. 68 (10): 930-941).

Methods for detecting TNF are disclosed in, e.g., U.S. Pat. No. 5,231,024A and can be utilized in the methods disclosed herein. Commercially available reagents can also be used, e.g., the Abcam: Human TNFα ELISA Kit (ab181421) and RayBiotech: Human TNF-α ELISA kit.

5.3.5.4. sTREM-2

TREM2 encodes a single-pass type I membrane protein that forms a receptor-signaling complex with the TYRO protein tyrosine kinase-binding protein (TYROBP) and thereby triggers the activation of immune responses in macrophages and dendritic cells (Paloneva et al., 2002, Am J Hum Genet. 71:656-62).

A proteolytic product of TREM2, referred to as soluble TREM2 (sTREM2), is abundant in the cerebrospinal fluid and its levels positively correlate with neuronal injury markers (Zhong et al., 2010, Nature Communications 10: Article 1365). Homozygous loss-of-function mutations in TREM2 cause Nasu-Hakola disease. Heterozygous rare variants, including those that cause Nasu-Hakola disease in the homozygous state, predispose to Alzheimer's disease, suggesting that the reduced function of TREM2 is key to the pathogenic effect of risk variants associated with Alzheimer's disease (Guerreiro et al., 2013, New England Journal of Medicine 368(2):117-127).

Methods for detecting sTREM-2 are disclosed in, e.g., WO2017062672A2 and can be utilized in the methods disclosed herein. Commercially available reagents can also be used, e.g., the Abcam Human TREM2 ELISA Kit (ab224881) and the Aviva systems biology: TREM2 ELISA Kit (Human) (OKBB01174).

5.3.5.5. Heat Shock Proteins

Heat shock proteins (HSPs) are a class of molecular chaperones that bind with nonnative proteins and assist them to acquire native structure and thus prevent misfolding and the aggregation process during the conditions of stress. Evidence suggests that HSPs are regulators of neurodegenerative processes correlated with protein misfolding in the brains of AD patients, with Hsp60, Hsp70 and Hsp90, are believed to be particularly important (see, e.g., Campanella et al., 2018, Int J Mol Sci. 19(9):2603 and Lu et al., 2014, BioMed Research International 2014: Article ID 435203).

Chaperonopathies are pathological conditions in which chaperones that are abnormal in composition/structure (e.g., because of mutations or post-translational modifications), quantitative levels, location, or function, play an either primary or auxiliary etiopathogenic role, and AD some HSPs co-localize with intracellular NFTs and Aβ plaques in the extracellular space (see, e.g., Campanella et al., 2018, Int J Mol Sci. 19(9):2603 and Wyttenbach & Arrigo, Madame Curie Bioscience Database, Landes Bioscience; 2000-2013).

Methods for detecting heat shock proteins are disclosed in, e.g., U.S. Pat. No. 5,447,843 and can be utilized in the methods disclosed herein. Commercially available reagents can also be used, e.g., the Enzo HSP70 High Sensitivity ELISA kit (ADI-EKS-715) or StressXpress® HSP70 Alpha ELISA kit (SKT-108), the Enzo HSP90a (human) ELISA kit (ADI-EKS-895) or StressXpress® HSP90 Alpha ELISA kit (SKT-107), and the StressXpress® HSP60 Alpha ELISA kit (SKT-110) or RayBio® Human HSP60 ELISA Kit (ELH-HSP60-1).

5.3.5.6. YKL40

YKL40 or YKL-40, also known as Chitinase-3-like protein 1 (CHI3L1), is a secreted glycoprotein that is approximately 40 kDa in size and is encoded by the CHI3L1 gene. The name YKL-40 is derived from the three N-terminal amino acids present on the secreted form and its molecular mass. YKL-40 is expressed and secreted by various cell-types including macrophages, chondrocytes, fibroblast-like synovial cells, vascular smooth muscle cells, and hepatic stellate cells (Canto et al., 2015, Brain, 138:918-931). The biological function of YKL-40 is unclear, and it is not known to have a specific receptor, however its pattern of expression is associated with pathogenic processes related to inflammation, extracellular tissue remodeling, fibrosis, asthma and solid carcinomas (Kazakova et al., 2009, Folia Medica. 51(1):5-14) and it is considered one of the most promising biomarkers of neuroinflammation in AD.

A meta-analysis confirmed elevated levels of YKL-40 in CSF and plasma of patients with AD dementia, although the association with AD was moderate compared with core AD biomarkers Aβ42, t-tau (total tau), and p-tau (phosphorylated tau or phospho-tau) (Olsson et al., 2016, Lancet Neurol. 15: 673-684).

Methods for detecting YKL-40 are disclosed in, e.g., US20130035290A1, US20140200184A1 and EP1804062A2 and can be utilized in the methods disclosed herein. Commercially available reagents can also be used, e.g., Abcam Human YKL-40 ELISA Kit (ab255719), 2. Clini Sciences Human CHI3L1/YKL-40 ELISA Kit (Sandwich ELISA), and Invitrogen CHI3L1/YKL-40 Human ELISA Kit.

5.3.6. Other Markers

The protein markers utilized in determining the risk score can further comprise one or more markers other than a tau peptide marker, an amyloid peptide marker, a neurodegeneration marker, a metabolic disease marker, and an inflammation marker (“other markers”), for example a frontotemporal lobe dementia (FTLD) marker (e.g., α-synuclein) or a Parkinson's Disease marker and/or a Lewy Body dementia marker (e.g., TDP-43), or other proteinopathy marker.

5.3.6.1. α-Synuclein

The human α-synuclein protein is made of 140 amino acids that, in humans, is encoded by the SNCA gene. It is abundant in the brain, while smaller amounts are found in the heart, muscle and other tissues. In the brain, α-synuclein is found mainly at the tips of neurons in specialized structures called presynaptic terminals. Within these structures, α-synuclein interacts with phospholipids and proteins (Sun et al., 2019, PNAS 116 (23): 11113-11115).

Although the function of α-synuclein is not well understood, studies suggest that it plays a role in restricting the mobility of synaptic vesicles, consequently attenuating synaptic vesicle recycling and neurotransmitter release (Larsen et al., 2006, Journal of Neuroscience 26 (46): 11915-22).

A 35-amino acid peptide fragment of α-synuclein is found in amyloid plaques and is known as the non-Aβ component (NAC) of Alzheimer's disease amyloid (U6da et al., 1993, PNAS 90 (23):11282-6 and Jensen et al., 1995, Biochem J 310 (1): 91-94). It has been suggested that this peptide is amyloidogenic and could promote the formation of β-amyloid in vivo (Bisaglia et al., 2006, Protein Science 15:1408-1416).

Methods for detecting α-synuclein are disclosed in, e.g., US20160077111A1 and EP1476758B2 and can be utilized in the methods disclosed herein. Commercially available reagents can also be used, e.g., the SNCA ELISA Kit and Abcam: Human α-synuclein ELISA Kit (ab260052).

5.3.6.2. TDP-43

The TAR DNA binding protein of 43 kDa (TDP43) is a highly conserved and ubiquitously expressed nuclear protein with roles in transcription and splicing regulation. It is also the major component of ubiquitin-positive cytoplasmic inclusions found in the brains of patients with frontotemporal lobar degeneration (FTLD) and amyotrophic lateral sclerosis (ALS).

In addition, TDP43-containing aggregates are found in a significant number of patients with Alzheimer's Disease (AD) and other neuromuscular disorders (Tremblay et al., 2011, Journal of neuropathology and experimental neurology, 70(9), 788-798).

Methods for detecting TDP43 are disclosed in, e.g., WO2016053610A1 and CA2853412A1 and can be utilized in the methods disclosed herein. Commercially available reagents can also be used, e.g., the Innoprot: TDP-43 Stress Granules Assay Cell Line.

### 5.4. Genetic Markers

5.4.1. Autosomal Dominant AD

Familial early-onset AD (EOAD) is usually caused by an autosomal dominant mutation in one of three genes: Presenilin 1 PSEN1 (chromosome 14), Presenilin 2 PSEN2 (chromosome 1), or Amyloid precursor protein APP (chromosome 21) (Skeehan et al., 2010, Genetics in medicine 12 (4 Suppl), S71-S82). A person with one of these fully penetrant mutations will contract the disease if they live long enough, usually developing symptoms before age 60. A very small percentage of AD cases arise in family clusters with early onset. Together, mutations in these genes explain 5-10% of the occurrence of early-onset AD. The identification of mutations in these genes has not only provided important insights in the molecular mechanisms and pathways involved in AD pathogenesis but also led to valuable targets currently used in diagnosis and drug development.

5.4.1.1. Amyloid Precursor Protein

Amyloid precursor protein (APP) is proteolytically processed by α-, β-, and γ-secretases following two pathways: the constitutive (nonamyloidogenic) or amyloidogenic pathway, leading to the production of different peptides.

In the amyloidogenic pathway, enriched in neurons, the subsequent proteolysis of APP by β-secretase and γ-secretase gives rise to a mixture of Aβ peptides with different lengths. The Aβ1-42 fragments are more aggregation-prone and are predominantly present in amyloid plaques in brains of AD patients.

A total of 39 APP mutations in 93 families are described, all of which affect proteolysis of APP in favor of Aβ1-42 (Cruts et al., 2012, Hum Mutat., 33:1340-1344). In addition, APP duplications have been identified in autosomal dominant early-onset families (Rovelet-Lecrux et al., 2006, Nat Genet., 38: 24-26).

APP mutations can be detected using. Athena Diagnostics: ADmark® APP DNA Sequencing Test and Duplication Test https://www.athenadiagnostics.com/view-full-catalog/a/admark-reg;-app-dna-sequencing-duplication-test

5.4.1.2. Presenilin 1 and 2

PSEN1 and PSEN2 are highly homologous genes. Mutations in PSEN1 are the most frequent cause of autosomal dominant AD known to date, whereas PSEN2 mutations are least frequent (Sherrington et al., 1995, Nature 375:754-760; St George-Hyslop et al., 1992, Nat. Genet., 2:330-334; Van Broeckhoven et al., 1992, Nat. Genet 2:335-339).

Both proteins are essential components of the γ-secretase complex, which catalyzes the cleavage of membrane proteins, including APP. Mutations in PSEN1 and PSEN2 impair the γ-secretase mediated cleavage of APP in Aβ fragments, resulting in an increased ratio of Aβ1-42 to Aβ1-40, either through an increased Aβ1-42 production or decreased Aβ1-40 production, or a combination of both (Cruts et al., 1998, Hum. Mutat. 11:183-190).

PSEN1 mutations cause the most severe forms of AD with complete penetrance, and the onset of disease can occur as early as 25 years of age (Cruts et al., 2012. Hum. Mutat. 33:1340-1344). The PSEN1 mutations have a wide variability of onset age (25-65 years), rate of progression, and disease severity. In comparison to PSEN1 mutations, PSEN2 mutation carriers show an older age of onset of disease (39-83 years), but the onset age is highly variable among PSEN2-affected family members (Sherrington et al., 1995, Nature 375:754-760; Sherrington et al., 1996, Hum. Mol. Genet. 5:985-988).

Mutations in the presenillin 1 gene can be detected using, e.g., the Athena Diagnostics: ADmark® PSEN1 DNA Sequencing Test and mutations in the presenillin 2 gene can be detected using, e.g., the Athena Diagnostics: ADmark® PSEN2 DNA Sequencing Test.

Various kits are available to detect mutations in APP, presenillin 1 and presenilin2, e.g., Athena Diagnostics: ADmark® Early Onset Alzheimer's Evaluation and the Invitae: Hereditary Alzheimer's Disease Panel.

5.4.2. ε4 Allele of Apolipoprotein E

The vast majority of people who develop AD have the late-onset form (LOAD), which has only one clearly established and robust genetic risk factor known as APOE or APO E (the gene that encodes the protein apolipoprotein E) (Saunders et al., 1993, Neurology; 43:1467-1472).

APOE encodes a polymorphic glycoprotein expressed in liver, brain, macrophages, and monocytes. APOE participates in transport of cholesterol and other lipids and is involved in neuronal growth, repair response to tissue injury, nerve regeneration, immunoregulation, and activation of lipolytic enzymes.

The APOE gene contains three major allelic variants at a single gene locus (ε2, ε3, and ε4), encoding for different isoforms (APO E2, APO E3, and APO E4, respectively) that differ in two sites of the amino acid sequence (Corder et al., 1993, Science 261:921-923).

The APOE ε4 allele increases risk in familial and sporadic early-onset and late-onset AD, but it is not sufficient to cause disease. The risk effect is estimated to be threefold for heterozygous carriers (APOE ε34) and 15-fold for ε4 homozygous carriers (APOE ε44), (Saunders et al., 1993, Neurology; 43:1467-1472) and has a dose-dependent effect on onset age. The APOE ε2 allele is thought to have a protective effect and to delay onset age (Farrer et al., 1997, JAMA; 278:1349-1356.)

Only 20-25% of the general population carries one or more ε4 alleles, where 40-65% of AD patients are ε4 carriers. The effect of the APOE ε4 allele accounts for 27.3% of the estimated disease heritability of 80% (Lambert et al., 2013, Nat. Genet; 45: 1452-1458).

The Athena Diagnostics ADmark® ApoE Genotype Analysis and Interpretation (Symptomatic) and the LabCorp: APOE Alzheimer's Risk tests can be used to identify a subject's APOE4 allele.

5.4.3. Additional Genetic Markers

Additional genetic factors have been identified for AD: Clusterin (CLU), Sortilin-related receptor-1 (SORL1), ATP-binding cassette subfamily A member 7 (ABCA7) (Van Clusterin (CLU) is a pleiotropic chaperone molecule that might be involved in AD pathogenesis through lipid transport, inflammation, and directly by influencing Aβ aggregation and clearance from the brain by endocytosis. A number of CLU variants exist and can have independent effects on the disease.

SORL1 was identified as a risk factor for late-onset AD through a candidate gene approach. Nonsense and missense mutations have been found in AD patients, including a common variant, which segregated with disease and affected APP processing in vitro.

ABCA7 was first identified as a risk gene for AD and is highly expressed in hippocampal neurons, one of the earliest affected brain regions of AD patients, and in microglia. An increased frequency of rare loss-of-function mutations in ABCA7 has been described in AD patients that may present with an autosomal dominant pattern of inheritance.

Methods for detecting genetic variations are known in the art, for example (i) sequencing methods, hybridization reactions between a target nucleic acid and allele-specific oligonucleotide (ASO) probes (see, e.g., European Patent Publications EP237362 and EP329311), (ii) allele specific amplification (see, e.g., U.S. Pat. Nos. 5,521,301; 5,639,611; and 5,981,176), (iii) quantitative RT-PCR methods (e.g., TaqMan assays; see, e.g., U.S. Pat. Nos. 5,210,015; 5,538,848; and 5,863,736), and (iv) various single base pair extension (SBPE) assays. Any of the foregoing methods and other known in the art can be used to detect mutations in CLU, SORL1 and ABCA7.

### 5.5. Computer Based Methods and Systems

5.5.1. Computer Based Methods and Systems for Assessing a Subject's Risk for Developing or Already Having AD

The present disclosure provides computer implemented methods for assessing a subject's risk for developing or already having AD, e.g., using the methods described in Section 5.2.

In some aspects, the methods can comprise executing, in a computer system having one or more processors coupled to a memory storing one or more computer readable instructions for execution by the one or more processors, the one or more computer readable instructions comprising instructions for (a) storing a dataset associated with the subject, wherein said dataset comprises quantitative data for at least 4 protein markers in one or more fluid samples from the subject, and (b) generating an AD risk score for the subject from the dataset, e.g., using a statistics- and/or artificial intelligence-based algorithm. The dataset can be stored on local server or on a remote server, e.g., in the cloud.

The computer-based methods can comprise generating a single AD risk score or, alternatively, multiple AD risk scores. For example, a single dataset can be used to generate two or more, three or more, four or more, five or more, or six or more individual AD risk scores that individually predict (i) the subject's brain amyloid load, (ii) the subject's brain tau load, (iii) brain neurodegeneration in the subject, or (iv) whether the subject exhibits symptoms sufficient for a diagnosis of mild cognitive impairment or AD. In some embodiments, the methods comprise generating one or more AD risk score that predict the subject's brain amyloid load, one or more AD risk score that predict the subject's brain tau load, one or more AD risk score that predict brain neurodegeneration in the subject, and one or more AD risk score that predict whether the subject exhibits symptoms sufficient for a diagnosis of mild cognitive impairment or AD. The predictions can be reported, for example, as a binary prediction (e.g., positive or negative). For example, when an AD risk score predicts that a subject is likely to have a brain amyloid load (at the time the fluid samples are obtained) at or above a cutoff value (e.g., a celtiloid value of 21), the risk score can be reported as “positive.” Conversely, when an AD risk score predicts that a subject's brain amyloid load is likely to be below the cutoff value, the risk score can be reported as “negative.” Similar binary predictions can be reported for AD risk scores that predict whether a subject is likely to have a brain tau load above a cutoff value, whether a subject is likely to have brain neurodegeneration, and whether a subject is likely to have symptoms sufficient for a diagnosis of mild cognitive impairment or AD.

In some aspects, the methods can comprise, in a computer system having one or more processors coupled to a memory storing one or more computer readable instructions for execution by the one or more processors, the one or more computer readable instructions comprising instructions for (a) storing a dataset comprising a plurality of patient records, each patient record comprising quantitative data for at least 4 or at least 5 protein markers in one or more fluid samples from the subject and (b) generating an AD risk score for the subject using a weighted scoring system for the at least 4 or at least 5 protein markers, e.g., using a statistics- and/or artificial intelligence-based algorithm. The dataset can be stored on local server or on a remote server, e.g., in the cloud.

The present disclosure further provides a system configured to generate an AD risk score according to any one of the computer implemented methods disclosed herein. The system typically comprises one or more processors coupled to a memory storing one or more computer readable instructions for execution by the one or more processors.

In some embodiments, the one or more computer readable instructions can comprise instructions for (a) storing a dataset associated with a subject, wherein said dataset comprises quantitative data for at least 4 protein markers in one or more fluid samples from the subject (e.g., blood samples such as plasma samples) and the protein markers comprise at least 3 of a tau peptide marker, an amyloid peptide marker, a neurodegeneration marker, a metabolic marker, and an inflammation marker; and (b) generating an AD risk score for the subject from the dataset.

In other embodiments, the one or more computer readable instructions can comprise instructions for (a) storing a dataset comprising a plurality of patient records, each patient record comprising quantitative data for at least 4 or at least 5 protein markers in one or more fluid samples from the subject, wherein: (i) the fluid samples are selected from blood, serum and cerebral spinal fluid (CSF); and/or (ii) the protein markers comprise at least 4 of a tau peptide marker, an amyloid peptide marker, a neurodegeneration marker, a diabetes and/or metabolic marker, and an inflammation marker; and (b) generating an AD risk score for the subject using a weighted scoring system for the at least 4 or at least 5 protein markers.

The AD risk score can be generated using a statistics- and/or artificial intelligence-based algorithm.

The systems and methods disclosed herein can be incorporated into a software tool accessed by medical practitioners to evaluate or analyze a subject's risk of developing (or already having developed) AD. In addition, medical practitioners may use the software tool to monitor subjects previously identified as having a moderate or high risk of developing AD. The software tool may also be used to identify subjects in need of an AD therapeutic and/or who might be recruited for a clinical trial of a candidate AD therapeutic.

For example, the software tool may be incorporated at least partially into a computer system used by a medical practitioner or other user. The computer system may receive marker data obtained from the subject (e.g., data concerning the levels of at least 4 or at least 5 protein markers in one or more fluid markers from a subject) as well as other relevant information (e.g., the subject's genetic marker status, the subject's family history of AD, the subject's date of birth or age, the subject's gender, the subject's education, the subject's performance on a cognitive assessment, the subject's prior risk scores, or any combination thereof). For example, the data may be input by the medical practitioner or may be received over a network, such as the Internet, from another source capable of accessing and providing such data, such as a medical lab, or a combination thereof. The data may be transmitted via a network or other system for communicating the data, directly into the computer system, or a combination thereof. The software tool may use the data to generate an AD risk score and may use the risk score, alone or in combination with other information concerning the subject, to provide recommendations for further testing or monitoring a subject. For subjects with a high risk score, the medical practitioner may provide further inputs to the computer system to select possible treatment options.

Alternatively, the software tool may be provided as part of a web-based service or other service, e.g., a service provided by an entity that is separate from the medical practitioner. The service provider may, for example, operate the web-based service and may provide a web portal or other web-based application (e.g., run on a server or other computer system operated by the service provider) that is accessible to medical practitioners or other users via a network or other methods of communicating data between computer systems.

For example, the data from a subject may be provided to the service provider, and the service provider may generate an AD risk score of the subject. Then, the web-based service may transmit an AD risk score (and optionally information and/or recommendations based on the subject's risk score and other relevant information) to the medical practitioner's computer system or display an AD risk score (and optionally information and/or recommendations based on the subject's risk score and other relevant information) to the medical practitioner. The medical practitioner may provide further inputs, e.g., to select possible treatment options or make other adjustments to the computational analysis (e.g., by entering other relevant information such as the subject's genetic marker status, the subject's family history of AD, the subject's date of birth or age, the subject's gender, the subject's education, the subject's performance on a cognitive assessment, the subject's prior risk scores, or any combination thereof), and the inputs may be transmitted to the computer system operated by the service provider (e.g., via the web portal).

One or more of the steps described herein may be performed by one or more human operators (e.g., a neurologist or other medical practitioner, the subject an employee of the service provider providing the web-based service or other service provided by a third party (e.g., a medical laboratory), other user, etc.), or one or more computer systems used by such human operator(s), such as a desktop or portable computer, a workstation, a server, a personal digital assistant, etc. The computer system(s) may be connected via a network or other method of communicating data.

Reports may also be generated using a combination of any of the features set forth herein. More broadly, any aspect set forth in any embodiment may be used with any other embodiment set forth herein.

5.5.2. Computer Based Methods and Systems for Producing an AI-Based Algorithm for Generating an AD Risk Score

The present disclosure provides computer implemented methods for produce an AI-based algorithm for generating an AD risk score. The AI-based algorithms for generating an AD risk score of the disclosure can be used to generate an AD risk score for a subject, e.g., using the methods described in Section 5.2 or 5.5.1.

In some aspects, the methods comprise executing, in a computer system having one or more processors coupled to a memory storing one or more computer readable instructions for execution by the one or more processors, the one or more computer readable instructions comprising instructions for (a) storing a dataset comprising a plurality of patient records, each patient record comprising quantitative data for at least 4 protein markers in one or more fluid samples from the patient and data for one or more AD surrogate variables for the patient and (b) training a machine learning model with at least a portion of the patient records to produce an algorithm that correlates quantitative data for the at least 4 protein markers to the one or more AD surrogate variables. Thus, when training the machine learning model, the quantitative data for at least 4 protein markers in the dataset can be used as the input variables, while the data for the one or more AD surrogate variables in the dataset can be used as the output variables. The patient records can also include additional information that can be used as input variables, for example, age, gender, education level, genetic risk markers for AD (e.g., APO E4, Clusterin (CLU), Sortilin-related receptor-1 (SORL1), ATP-binding cassette subfamily A member 7 (ABCA7)), and age at which the patient received a tau scan (age at tau scan).

An AD surrogate variable is a factor linked to AD risk, for example brain amyloid load, brain tau load, brain neurodegeneration, or clinical diagnosis of mild cognitive impairment (MCI) or AD. Thus, for example, an algorithm designed with brain amyloid load as an AD surrogate variable can be used to predict a subject's brain amyloid load and, by extension, the subject's risk for developing or already having AD. An AD surrogate variable can be associated with a high, medium, or low risk of developing AD. Exemplary features of AD surrogate variables are described in further detail in Sections 5.5.2.1 to 5.5.2.4.

The plurality of patient records can include at least 100 patient records, at least 200 patient records, at least 300 patient records, at least 500 patient records, at least 1000 patient records, at least 5000 patient records, or more. The number of patient records used to train the machine learning model can be at least 100, at least 200, at least 300, at least 500, at least 1000, at least 5000, or more than 5000. A portion of a given plurality of patient records can be used to train the machine learning model (training set), while another portion can be used to test the produced algorithm (testing set). For example, 70%-75% of the patient records may be used as the training set, while 25%-30% of the patient records may be used as the testing set.

The data in each individual patient record is preferably obtained at approximately the same time. For example, quantitative data for the protein markers is preferably can be obtained from the same blood or CSF draw. In some instances, there may be a small period of time between obtaining different data in a patient record. For example, protein biomarkers may be determined from a blood draw obtained prior to an amyloid or tau PET scan. Preferably, all data in an individual patient record is data obtained within a period of 12 weeks or less, e.g., 10 weeks, 8 weeks, 6 weeks, 4 weeks, 2 weeks, or 1 week.

The methods can further comprise a step of tuning hyperparameters of the model to optimize the hyperparameters.

The methods can further comprise a step of retraining the model with updated patient records, e.g., patient records comprising quantitative data for the protein markers and one or more AD surrogate variables obtained six months, one year, or more than one year after the initial quantitative data for the protein markers and one or more AD surrogate variables included were obtained.

Exemplary machine learning models that can be used include logistic regression, light GBM, Random Forest, and CatBoost models. Other machine learning models can also be used, for example linear discriminant analysis, Adaptive Boosting, Extreme Gradient Boosting, Extra Trees, Naïve-Bayes, K-Nearest neighbor, Gradient Boosting, and Support Vector models. Blending can be used to combine predictions from two or more base models, e.g., two or more types of models identified in this paragraph.

Systems configured to produce an AI-based algorithm for generating an AD risk score can further include instructions for scoring a subject's risk for developing or already having AD using an AI-based algorithm produced by the system. Such systems can have two modes, a training mode for producing an AI-based algorithm for generating an AD risk score, and an AD risk score generating mode. The training mode can be used to generate an AI-based algorithm, and the AD risk score generating mode can be used to generate AD risk scores from subject datasets. Thus, once the system's training mode is used to generate an AI-based algorithm, the system can be used in AD risk score generating mode to score subject datasets.

In further aspects, the disclosure provides tangible, non-transitory computer-readable media that comprise instructions for one or more of the computer implemented methods described herein. Examples of non-transitory computer media include internal disks (e.g., hard drives) and removable disks (e.g., flash drives, DVDs, CDROMs, etc.)

5.5.2.1. Brain Amyloid Load

An AI-based AD risk score algorithm for predicting a subject's brain amyloid load and, by extension, the subject's risk for developing or already having AD, can be generated by using brain amyloid load as an AD surrogate variable. For example, in the plurality of patient records, patient data for brain amyloid load can include data for patient amyloid PET centiloid values (e.g., from PET brain imaging with [¹³F]flutemetamol or [¹⁸F]florbetapir). Thus, for example, training the machine learning model with quantitative data for the at least 4 protein markers as input variables and PET centiloid value data as output variable can produce an algorithm that can be used to predict, from a given subject's protein marker levels, whether the subject is likely to have a PET centiloid value above or below a cutoff value. Additional input variables, for example, age, gender, education level, genetic risk markers for AD (e.g., APO E4, Clusterin (CLU), Sortilin-related receptor-1 (SORL1), ATP-binding cassette subfamily A member 7 (ABCA7)), age at tau scan, and combinations thereof can also be included.

In some embodiments, the cutoff value is 12 (where a value less than 12 can predict a low risk of developing AD) or 21 (where a value greater than or equal to 21 can predict a high risk of having or developing AD. See, Salvado et al., 2019, Alzheimer's Research & Therapy, 11(1):1-12; Amadoru et al., 2020, Alzheimer's Research & Therapy 12(1):1-8. The use of two AD risk score algorithms with different cutoff values can be used to classify a subject as having a low, medium, or high risk of having or developing AD. For example, using a first AD risk score algorithm that predicts the likelihood that a subject has a PET centiloid value less than 12 (representing a low risk) and a second AD risk score algorithm that predicts the likelihood that a subject has a PET centiloid value greater than or equal to 21 (representing a high risk), a prediction of the likelihood of the subject having a PET centiloid value between 12 and 21 (representing a medium risk) can be made.

Other measures of brain amyloid load can also be used. For example, full brain amyloid standardized uptake value ratio (SUVR) or a volume of interest (VOI)-based amyloid standardized uptake value ratio (SUVR) or centiloid values, or AmyloidIQ data can be used.

In some embodiments, a Random Forest or CatBoost machine learning model is used to generate an AD risk score algorithm when an AD surrogate variable is brain amyloid load.

5.5.2.2. Brain Tau Load

An AI-based AD risk score algorithm for predicting a subject's brain tau load and, by extension, the subject's risk for developing or already having AD, can be generated by using brain tau load as an AD surrogate variable. For example, in the plurality of patient records, patient data for brain tau load can include data for patient tau PET standardized uptake value ratio (SUVR) values (e.g., from PET brain imaging with [¹⁸F] MK6240, Flortaucipir, RO948, Genentech Tau Probe (GTP) 1, PI-2620 or other PET tracer, for example a volume of interest (VOI) such as the mesial temporal region or temporal region). Thus, for example, training the machine learning model with quantitative data for the at least 4 protein markers as input variables and PET SUVR value data as output variable can produce an algorithm that can be used to predict, from a given subject's protein marker levels, whether the subject is likely to have a PET SUVR value above or below a cutoff value. Additional input variables, for example, age, gender, education level, genetic risk markers for AD (e.g., APO E4, Clusterin (CLU), Sortilin-related receptor-1 (SORL1), ATP-binding cassette subfamily A member 7 (ABCA7)), age at tau scan, and combinations thereof can also be included.

In some embodiments, the cutoff value is the 95^{th} percentile SUVR in healthy subjects (e.g., according to the methods described in Doré et al., 2021, European Journal of Nuclear Medicine and Molecular Imaging 48(7):2225-32). Brain tau PET SUVR above this cutoff value can be considered a high risk AD surrogate variable, such that a subject who is predicted to have a brain tau PET SUVR value above the cutoff can be classified as having a high risk for having or developing AD.

Mesial Temporal (“MT”) region is in some embodiments defined as the region comprising entorhinal cortex, hippocampus, para-hippocampus, and amygdala. Temporal region (“TJ”) can in some embodiments be defined as the temporal composite region described in Jack et al., 2018, Brain 141(5):1517-1528, the contents of which are incorporated herein by reference.

Measures of brain tau load other than SUVR in mesial temporal or temporal regions can also be used. For example, full brain tau standardized uptake value ratio (SUVR), another volume of interest (VOI)-based tau standardized uptake value ratio (SUVR), or the aforementioned brain regions measured using another standardized method for measuring brain Tau load (e.g., TauIQ) can be used.

In some embodiments, a CatBoost machine learning model is used to generate an AD risk score algorithm when an AD surrogate variable is brain tau load.

5.5.2.3. Brain Neurodegeneration

An AI-based AD risk score algorithm for predicting neurodegeneration in a subject and, by extension, the subject's risk for developing or already having AD, can be generated by using a neurodegeneration AD surrogate variable. For example, in the plurality of patient records, patient data for neurodegeneration can include data for patient clinical dementia rating (CDR) scores (Hughes et al., 1982, Br J Psychiatry 140:566-72). Thus, for example, training the machine learning model with quantitative data for the at least 4 protein markers as input variables and CDR score data as output variable can produce an algorithm that can be used to predict, from a given subject's protein marker levels, whether the subject is likely to have a CDR score indicative of neurodegeneration. Additional input variables, for example, age, gender, education level, genetic risk markers for AD (e.g., APO E4, Clusterin (CLU), Sortilin-related receptor-1 (SORL1), ATP-binding cassette subfamily A member 7 (ABCA7)), age at tau scan, and combinations thereof can also be included. In some embodiments, the CDR score indicative of neurodegeneration is 0.5. A CDR score at or above 0.5 can be considered a high risk AD surrogate variable, such that a subject who is predicted to have a CDR score at or above 0.5 can be classified as having a high risk for having or developing AD.

Measures of brain neurodegeneration other than CDR can also be used. For example, mini-mental state examination (MMSE), Montreal Cognitive Assessment (MOCA), Alzheimer's Disease Assessment Scale-Cognitive section (ADAS-Cog), Delis-Kaplan Executive Function System (D-KEFS), Addenbrookes Cognitive Assessment (ACE-R), reduced cortical thickness and grey and white matter hyperintensities can be used.

In some embodiments, a Light GBM machine learning model is used to generate an AD risk score algorithm when an AD surrogate variable is brain neurodegeneration.

5.5.2.4. Clinical Diagnosis

An AI-based AD risk score algorithm for predicting the likelihood of a subject exhibiting symptoms sufficient for a diagnosis of MCI or AD and, by extension, the subject's risk for developing or already having AD, can be generated by using clinical diagnosis of MCI or AD as an AD surrogate variable. For example, in the plurality of patient records, patient data for clinical diagnosis can include data for patient diagnosis status for MCI or AD. Thus, for example, training the machine learning model with quantitative data for the at least 4 protein markers as input variables and clinical diagnosis data as output variable can produce an algorithm that can be used to predict, from a given subject's protein marker levels, whether the subject is likely to have symptoms sufficient for a diagnosis of mild cognitive impairment or AD. Additional input variables, for example, age, gender, education level, genetic risk markers for AD (e.g., APO E4, Clusterin (CLU), Sortilin-related receptor-1 (SORL1), ATP-binding cassette subfamily A member 7 (ABCA7)), age at tau scan, and combinations thereof can also be included.

A clinical diagnosis AD surrogate variable can be considered a high risk AD surrogate variable, such that a subject who is predicted to have symptoms indicative of MCI or AD can be classified as having a high risk for having or developing AD.

In some embodiments, a Logistic Regression machine learning model is used to generate an AD risk score algorithm when an AD surrogate variable is clinical diagnosis of MCI or AD.

Various modifications and variations can be made in the disclosed systems and processes without departing from the scope of the disclosure. Other embodiments will be apparent to those skilled in the art from consideration of the specification and practice of the disclosure disclosed herein. It is intended that the specification and Examples below be considered as exemplary only.

### 6. EXAMPLES

### 6.1. Example 1: Development of PREFER-AD Predictive Fluid Biomarker Panel for Early Alzheimer's Disease Prognosis

6.1.1. Materials & Methods

A Predictive Fluid Biomarker Panel for Early Alzheimer's Disease Prognosis (PREFER-AD) is developed to predict the probability of older adults to advance to Alzheimer's Disease. Data from the Australian Imaging, Biomarker & Lifestyle Study of Ageing (AIBL) and other relevant studies are analyzed for fluid (plasma, whole serum and/or cerebral spinal fluid) biomarker status, which are related to observed progression to cognitive impairment in correlation with neurological diagnoses and relevant imaging studies (e.g., MRI, PET). The data utilized are derived from ≥350 subjects with approximately the following characteristics:
approximately 55% cognitively normal individuals
approximately 20% individuals diagnosed with Mild Cognitive Impairment
approximately 15% individuals diagnosed with Alzheimer's Disease
approximately 5% individuals diagnosed with Frontal Temporal Dementia or dementia of unknown cause, and
approximately 5% individuals with diagnoses pending/unknown.

The following data are associated with each subject:
Demographics (age, sex)
Clinical diagnosis
APOE status (if available)
Amyloid status
Neuropsychological assessments
Date of blood collection
[18F] MK-6240 PET scan
[18F] NAV-4694 PET scan

Fluid biomarkers that are analyzed for each subject include some or all of the following markers listed in Table 1.

TABLE 1

| Fluid Biomarker | Marker Category |
|---|---|
| p-tau 181 | Tau peptide |
| p-tau 217 | Tau peptide |
| Aβ - 40 | Amyloid peptide |
| Aβ - 42 | Amyloid peptide |
| Aβ - 40/42 ratio | Ratio of amyloid peptide |
| Neurofilament Light (NFL) | Neurodegeneration |
| HbA1c | Metabolic disorder and/or diabetes |
| C-Reactive Protein (CRP) | Inflammation marker |
| Interleukin-6 (IL-6) | Inflammation marker |
| Tumor Necrosis factor (TNF) | Inflammation marker |
| sTREM-2 | Inflammation marker |
| Heat shock protein | Inflammation marker |
| TDP-43 | Frontotemporal lobe |
| | dementia (FTLD) |
| α-Synuclein | Parkinson's disease and/or Lewy |
| | Body dementia |
| YKL-40 | Inflammation |
| Glial fibrillary acidic protein (GFAP) | Neurodegeneration |

These fluid biomarker analytes are assessed using advanced Elisa assays, such as are available on the Quanterix SIMOA® platform (or comparable research-oriented platforms) and/or where possible on clinically approved in vitro diagnostic analyzer platforms (e.g., Roche Cobas, Siemens Healthineers Centaur).

To generate the risk score, both classical statistical methods such as the partial least squares (PLS) regression and machine learning methods such as Random Forest are used.

In the case of PLS, the risk score is derived from the association between the measures of the fluid biomarker panel and the disease phenotypic measures such as the clinical diagnosis, MK-6240 Tau PET load, and neuropsychological assessments. The PLS is an iterative regression technique that performs least square regression on a smaller set of latent vectors derived from the predictors (fluid biomarker values) that maximizes the correlation between the outcome and the predictors. Variable Importance for Projection (VIP) will be used to identify the highly influential predictors (fluid biomarkers) by setting a cut-off at 0.8 for the VIP value.

To generate the risk score using the Random Forest regression model, an outcome variable is selected from the disease phenotypic measures as in the PLS method. Random Forest is a method that employs an ensemble of decision trees each trained with a different sample selected by bootstrapping sampling technique. By employing this technique, Random Forest reduces the model variance. It also employs a feature selection technique called ‘feature bagging’ by selecting only a random subset of features at each tree split to reduce the correlation between the decision trees in the ensemble. Feature importance metric from the Random Forest regressor will be used to identify the most influential biomarkers in the fluid biomarker panel.

All of the prediction models evaluated are trained employing k-fold cross validation technique to reduce sample bias and over fitting and would be tested with a left-out testing sample or an independent testing sample where possible. Performance of the combined risk score (average) from the all the prediction models is also evaluated, to investigate if the combination of multiple unrelated prediction models can provide a superior prediction compared to a single model.

6.1.2. Results

A mid to high-single digit number of fluid biomarkers are identified that collectively reliably predict the risk of developing Alzheimer's Disease. A weighted composite score based on these fluid biomarkers provides a risk profile, suggesting a course of management for the subject. This weighted composite score is scaled in a range of 1 to 10, where:
0-5 indicates no or very low risk of development of AD resulting in a re-test in e.g. 5 years,
>5-<8 indicates some risk of developing AD resulting in a re-test in e.g. 2 years, and
≥8-10 indicates considerable risk for development of/progression to/presence of AD needing immediate specialist attention.

### 6.2. Example 2: Case Studies of the Use of PREFER-AD

6.2.1. Case Study 1: Use of PREFER-AD to Predict AD Risk in Subject with Familial History

A 38 year-old male positive for APOE4 and a family (an aunt and great uncle) history of Alzheimer's Disease requests a risk assessment from his primary care physician (PCP). After running the PREFER-AD fluid biomarkers test, the PCP indicates an intermediate risk of progression to AD based on a score of 6. The PCP recommends a repeat assessment after two (2) years, or if the subject begins to exhibit subjective memory complaints.

6.2.2. Case Study 2: Use of PREFER-AD to Predict AD Risk in Subject During Standard Health Screening

A 55 year-old female executive reports subjective memory complaints at her annual health assessment. Her physician performs a PREFER-AD test and the subject receives a score of 2. She is encouraged to revisit any concerns after 4-5 years.

### 6.3. Example 3: Development of PREFER-AD Predictive Fluid Biomarker Panel for Early Alzheimer's Disease Prognosis Using Plasma Markers

A Predictive Fluid Biomarker Panel for Early Alzheimer's Disease Prognosis (PREFER-AD) was developed to assess the risk of adults having or advancing to Alzheimer's Disease.

6.3.1. Materials & Methods

6.3.1.1. Study Design

There is no known single variable that defines a subject's risk of AD. Hence, a set of surrogate variables were identified that are linked to AD risk and, using machine learning, each of these surrogate risk variables was modeled with plasma biomarkers as input variables. Age and gender were also included in the analysis as input variables.

6.3.1.2. Outcome (Surrogate) Variables

The identified set of surrogate variables can be categorized based on the category of the biomarker as follows:

TABLE 2

| | SURROGATE | AD RISK |
|---|---|---|
| CATEGORY | VARIABLE | CATEGORY |
| BRAIN | Amyloid PET Centiloid | Low |
| AMYLOID | Value less than 12 | |
| LOAD | Salvadó et al. 2019, | |
| | Alzheimer's Research & | |
| | Therapy 11(1): 1-12) | |
| | Amyloid PET Centiloid | Medium |
| | Value between | |
| | 12 and 21 (CL12) | |
| | Amyloid PET Centiloid | High |
| | Value greater than or | |
| | equal to 21 (CL21) | |
| | (Amadoru et al, 2020, | |
| | Alzheimer's Research & | |
| | Therapy 12(1): 1-8) | |
| BRAIN TAU | MK6240 Tau | High |
| LOAD | PET SUVR in | |
| | Mesial Temporal | |
| | (“MT”) Region >1.181 | |
| | MK6240 Tau | High |
| | PET SUVR in | |
| | Temporal (“TJ”) | |
| | Region >1.216 | |
| BRAIN | Clinical Dementia | High |
| NEURODEGENERATION | Rating (Sum of Boxes) | |
| (IMAGING AND | greater than or | |
| NEUROPSYCHOLOGICAL | equal to 0.5 | |
| ASSESSMENTS) | | |
| CLINICAL DIAGNOSIS | Diagnosis of MCI or AD | High |

Mesial Temporal (“MT”) region for brain Tau load was defined as the region comprising entorhinal cortex, hippocampus, para-hippocampus, and amygdala. Temporal region (“TJ”) is the temporal composite region described in Jack et al., 2018, Brain 141(5):1517-1528.

Processing methods and Centiloid values calculations for the Brain Amyloid PET images were as described in Bourgeat et al., 2018, Neurolmage 183:387-393. MK6240 Tau images were processed using the methods described in Doré et al., 2021, European Journal of Nuclear Medicine and Molecular Imaging 48(7):2225-32, with the cut-off values calculated based on the 95^{th} percentile of the amyloid negative participants in each composite volume of interest.

6.3.1.3. Blood Assay (Input) Variables

The blood plasma assay variables used as input variables for prediction of risk of AD were categorized by the pathology they represent, as shown in Table 3:

TABLE 3

| Plasma | | Measurement |
|---|---|---|
| Biomarker | Marker Category | Platform |
| p-tau 181 | Tau peptide | Quanterix |
| | | SIMOA ® |
| Aβ - 40 | Amyloid peptide | Quanterix |
| | | SIMOA ® |
| Aβ - 42 | Amyloid peptide | Quanterix |
| | | SIMOA ® |
| Neurofilament Light | Neurodegeneration | Quanterix |
| (NFL) | | SIMOA ® |
| Glial fibrillary | Neuroinflammation/ | Quanterix |
| acidic protein (GFAP) | Neurodegeneration | SIMOA ® |
| sTREM-2 | Inflammation marker | ELISA |
| α-Synuclein | Other proteinopathies | Quanterix |
| | (Parkinson's disease and/ | SIMOA ® |
| | or Lewy Body dementia) | |
| Adiponectin | Other proteinopathies/ | Quanterix |
| | metabolic marker | SIMOA ® |
| TDP-43 | Other proteinopathies | Quanterix |
| | (Frontotemporal | SIMOA ® |
| | lobe dementia (FTLD)) | |

6.3.1.1. Patient Records

The data utilized in this Example were derived from 363 subjects with approximately the following characteristics:
approximately 58% cognitively normal individuals
approximately 22% individuals diagnosed with Mild Cognitive Impairment
approximately 20% individuals diagnosed with Alzheimer's Disease

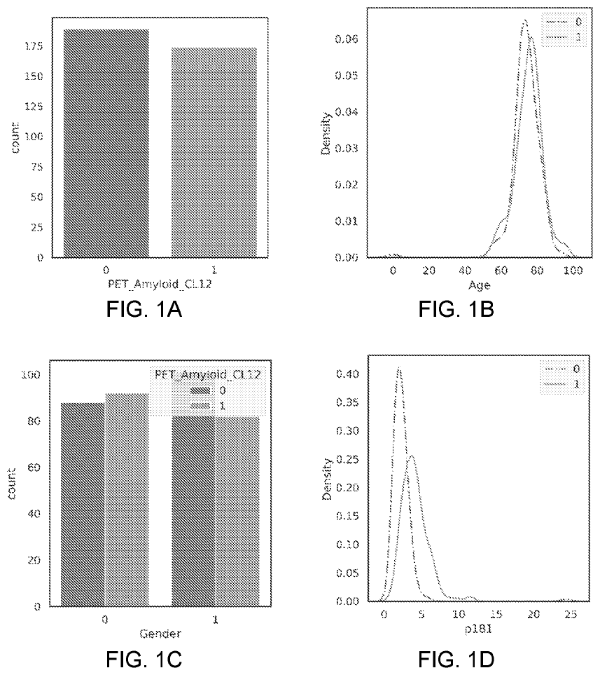

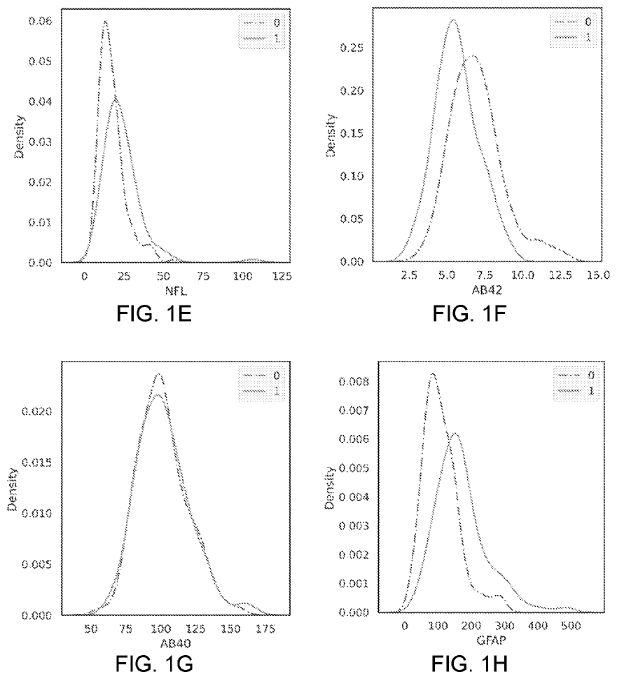

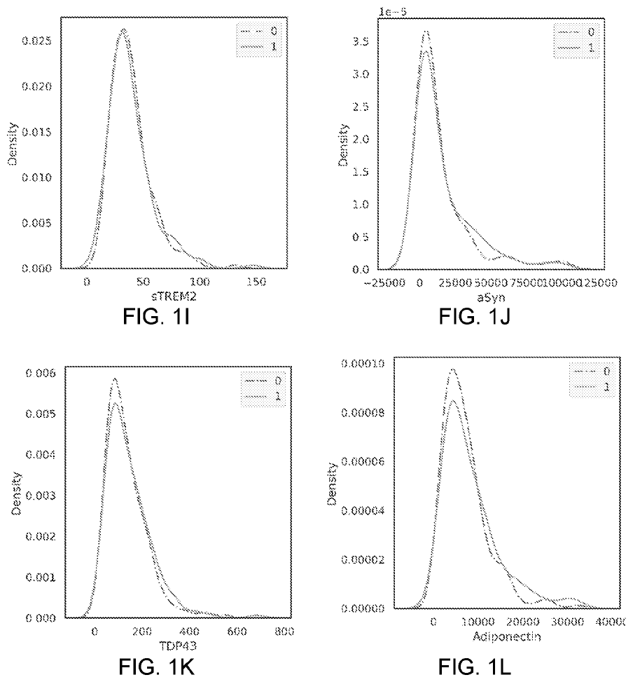

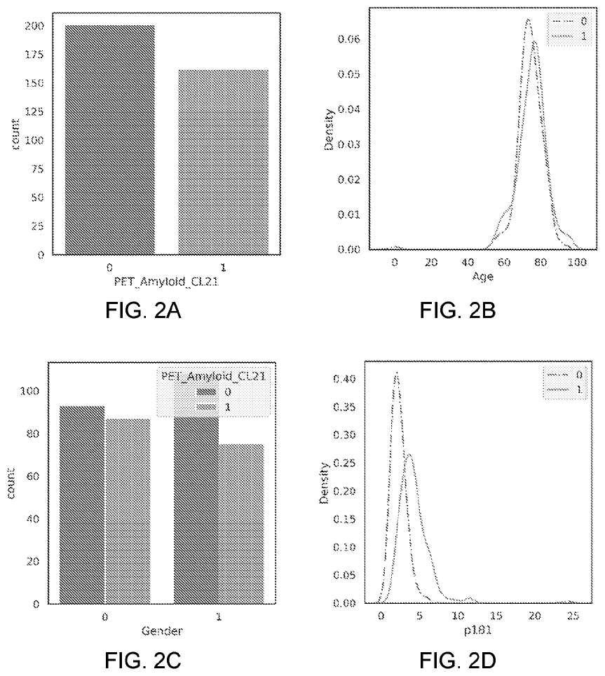

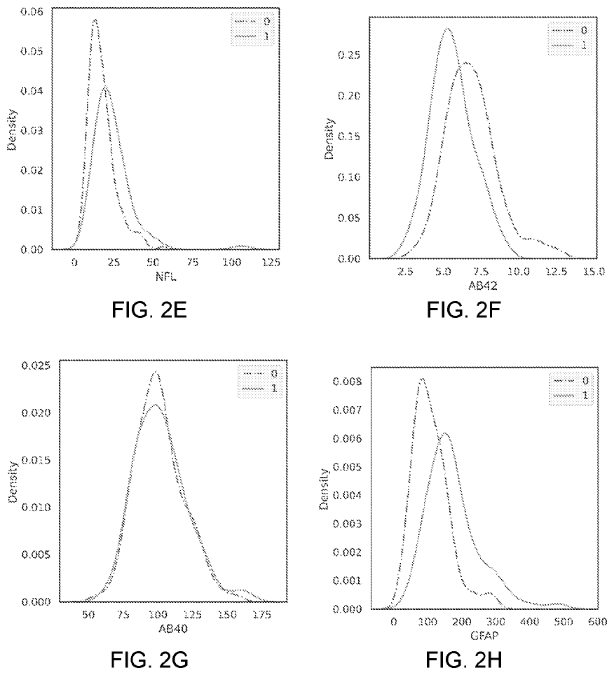

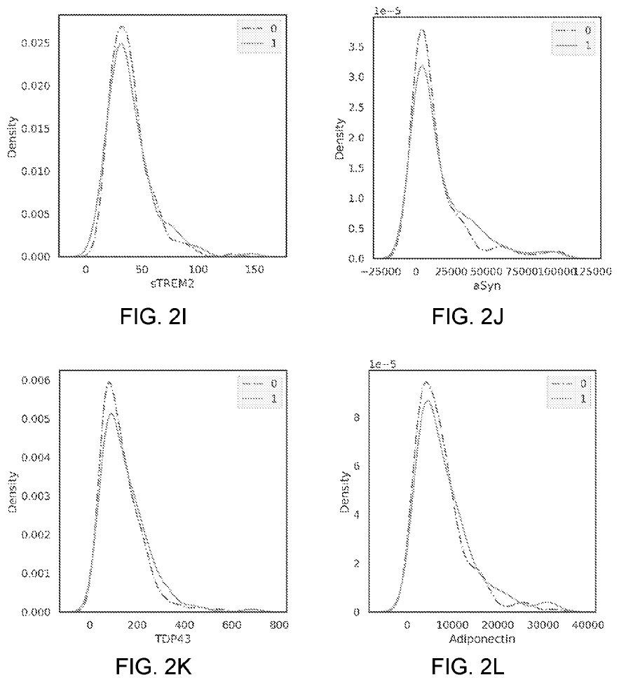

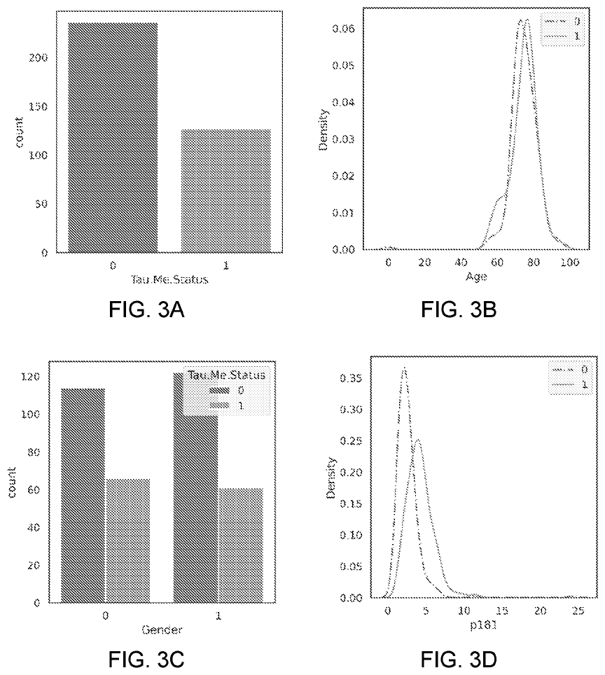

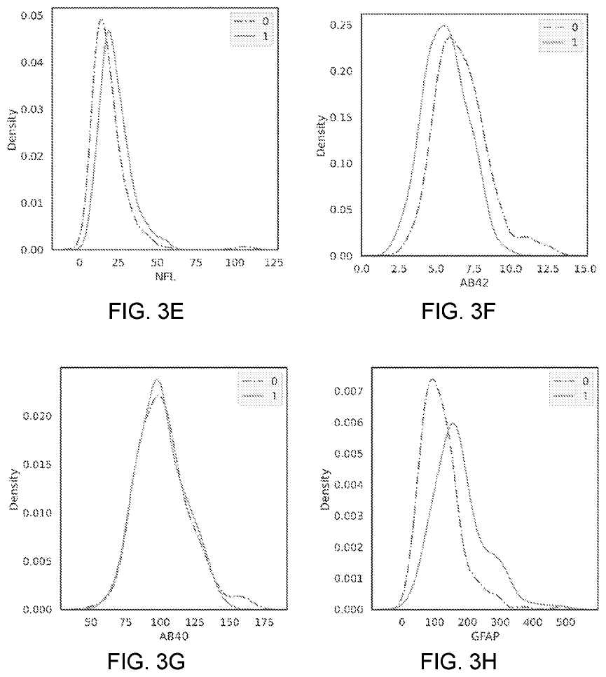

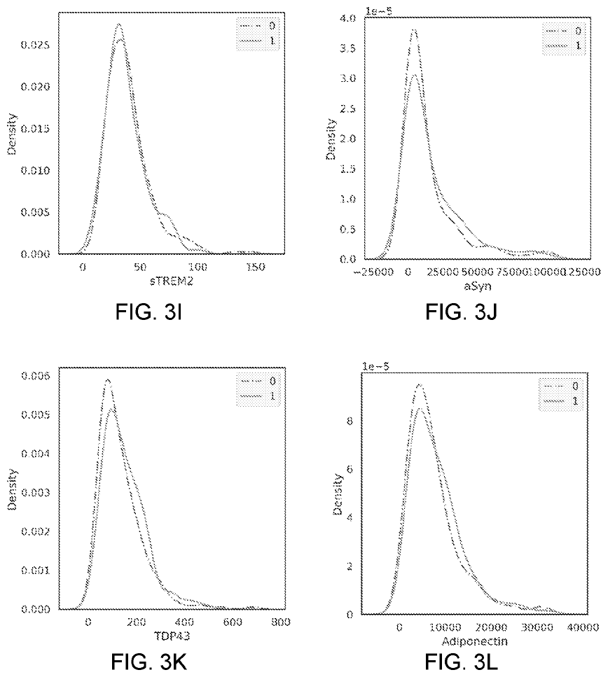

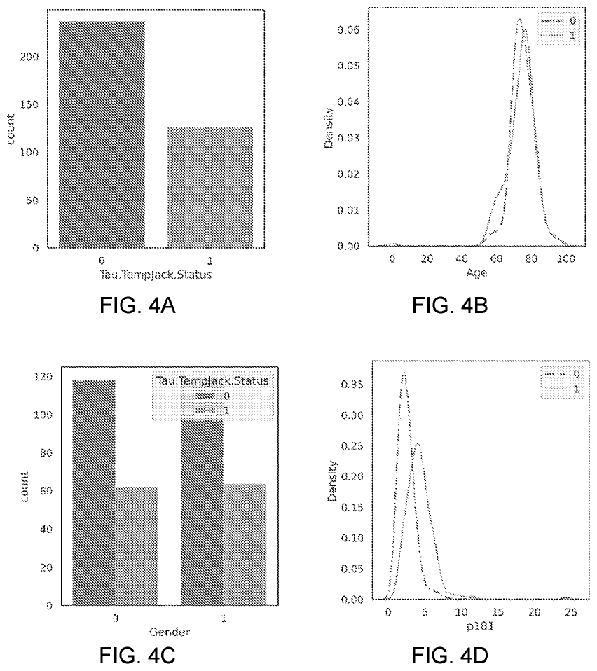

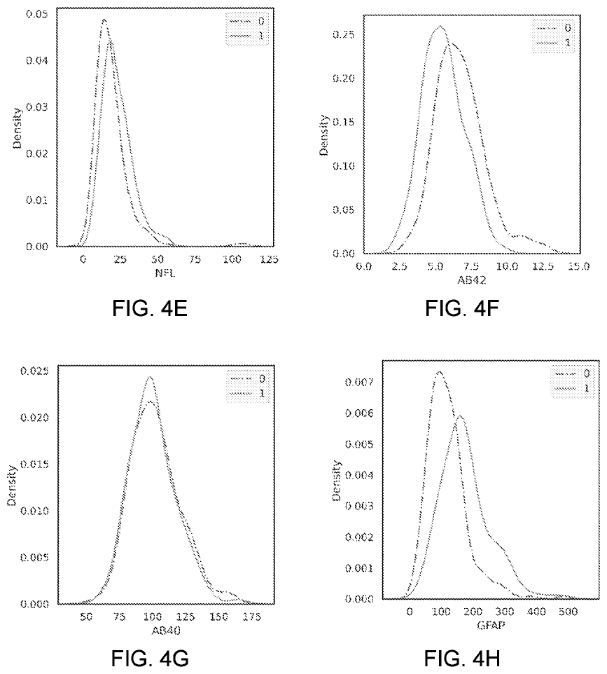

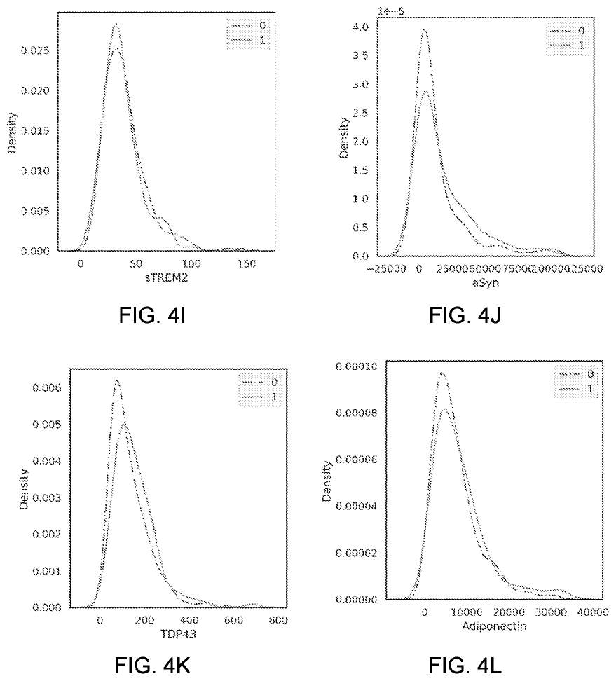

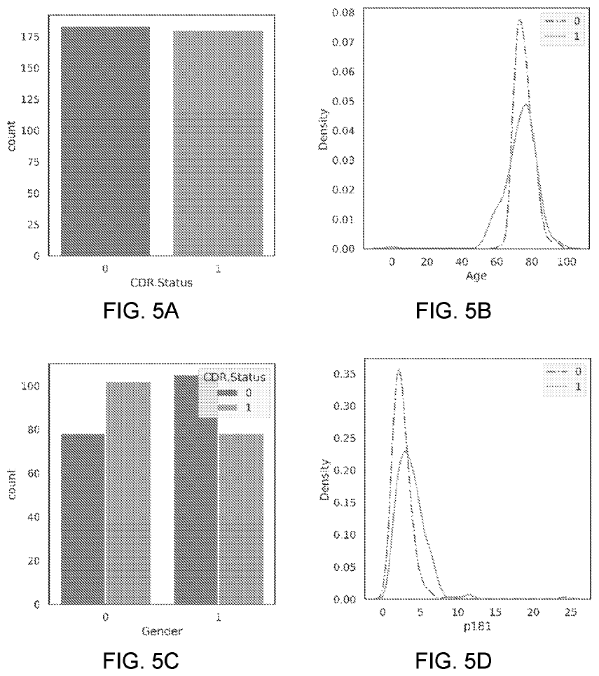

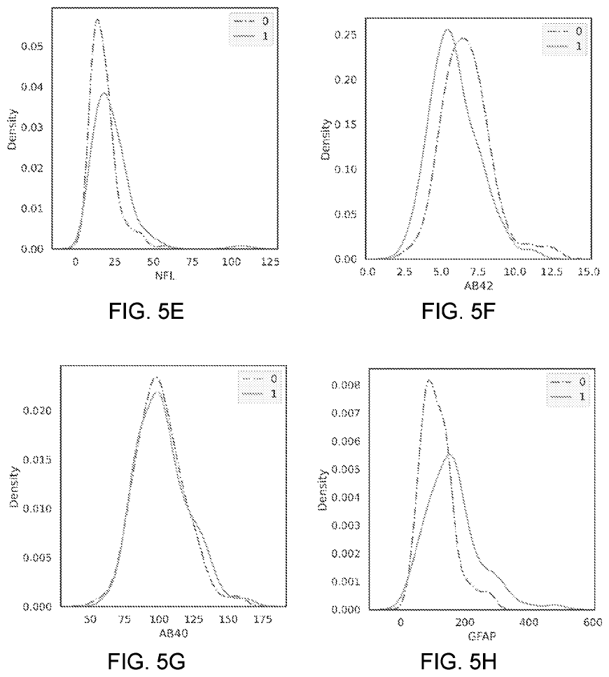

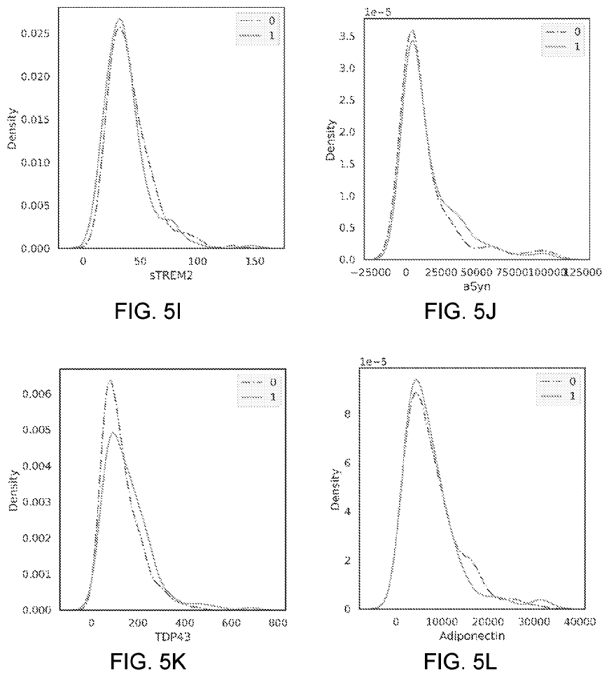

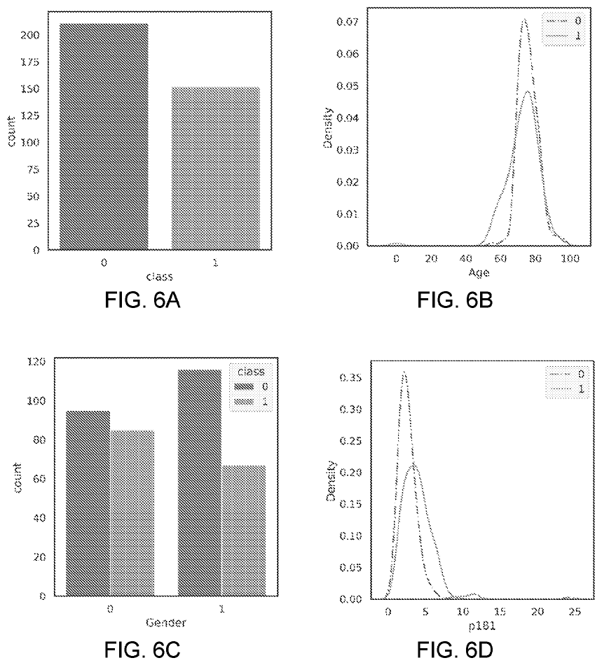

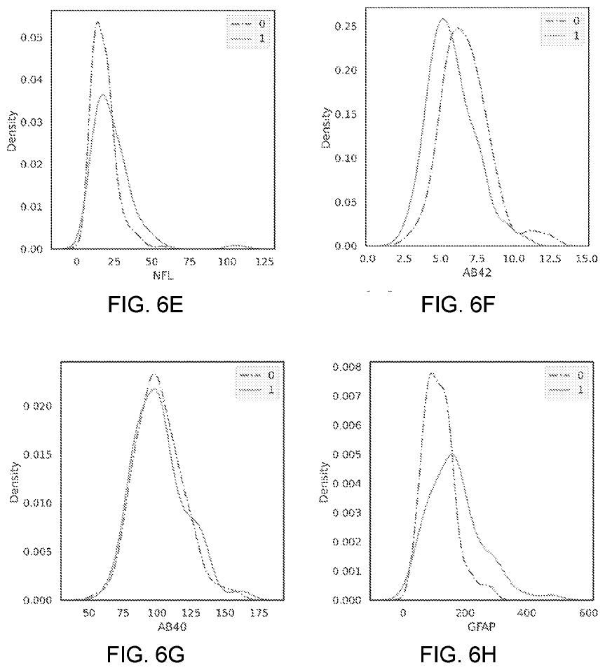

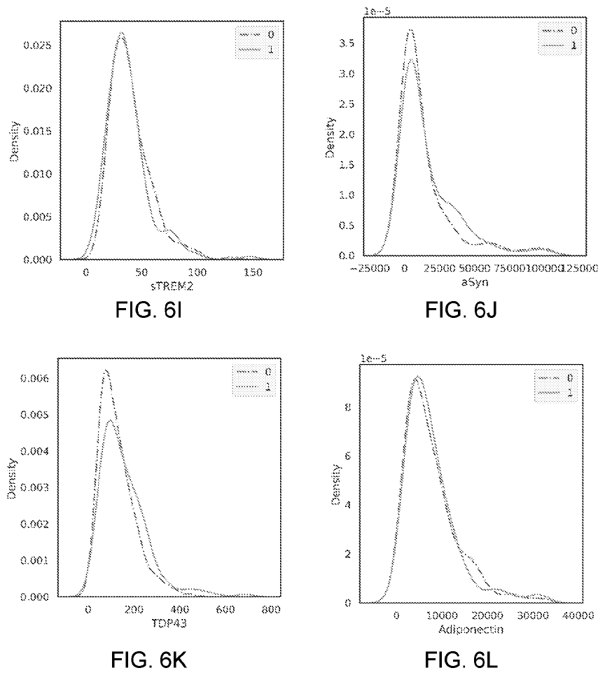

Descriptive statistics for the patient record data used in this Example are shown in FIGS. 1A-6L.

6.3.1.2. Machine Learning Analysis

For each of the surrogate variables, a collection of machine learning models (including CatBoost, Random Forest, Logistic Regression, Light GBM, Linear Discriminant Analysis) was employed to identify the best performing model. Blending of the foregoing models was also used. Since the number of cases in each outcome class were not equal the Area Under the Receiver Operating Characteristic Curve (AUCROC) and the Average Precision was used to compare performances.

10-fold cross-validation with 70% of the patient records were used to train the parameters and to find the optimal hyper-parameters for each model evaluated. 30% of the patient records were used for testing the models. Due to the limited number of samples available in the complete dataset, the best performing model was selected based on the average performance in the testing set.

6.3.2. Results

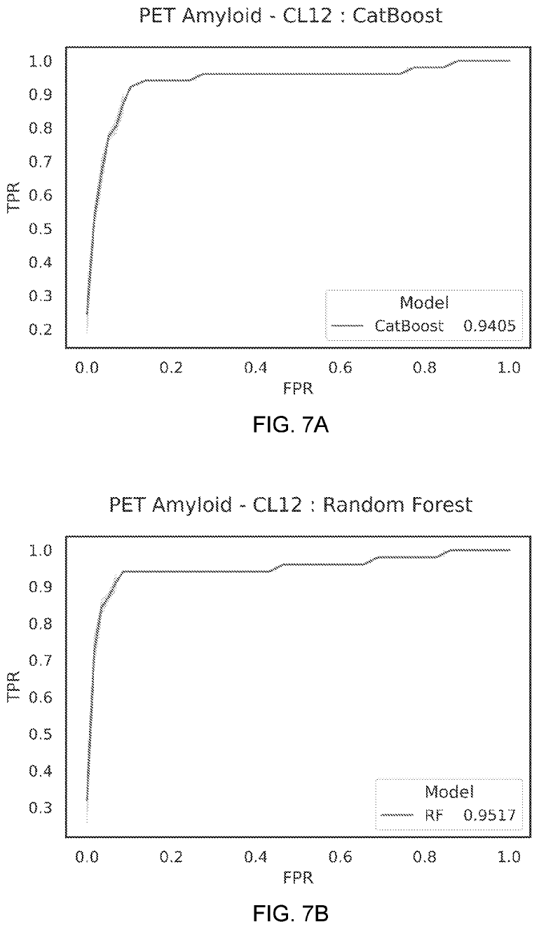

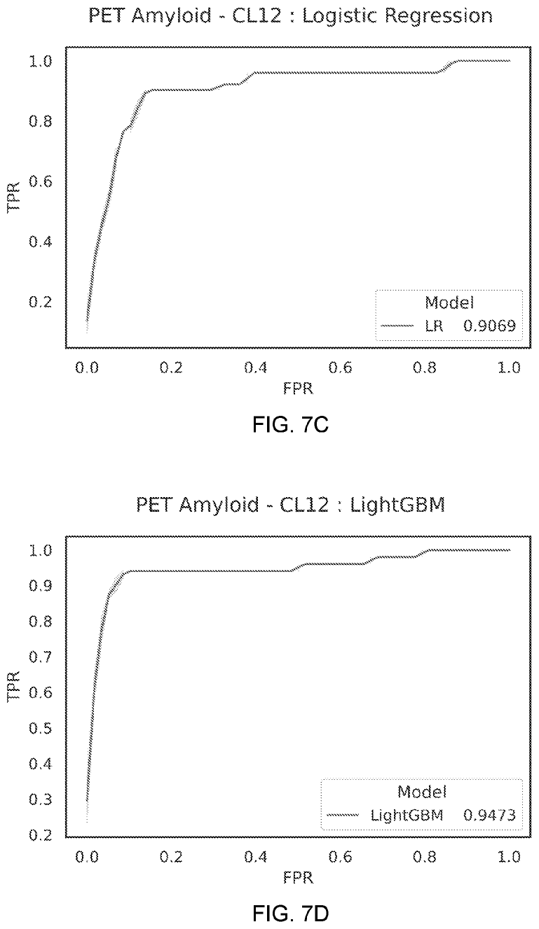

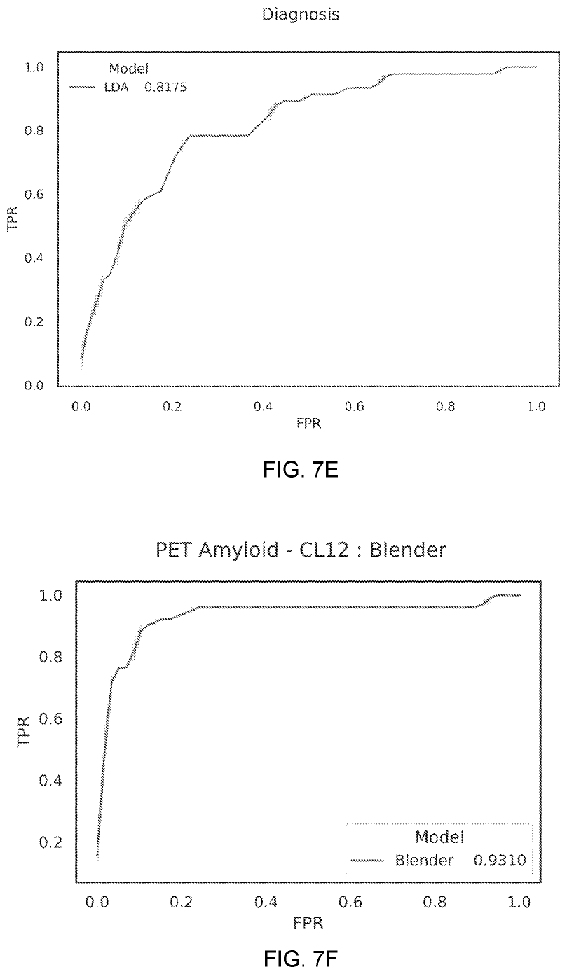

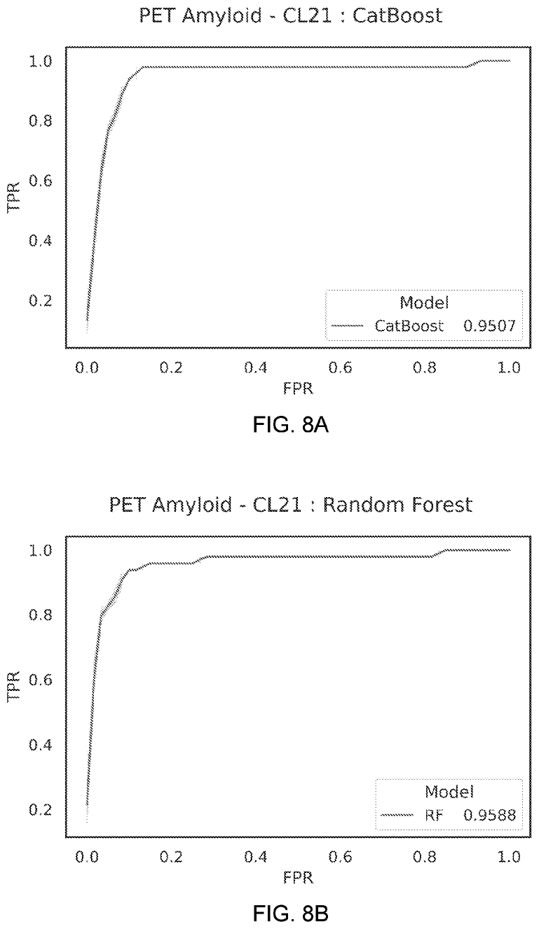

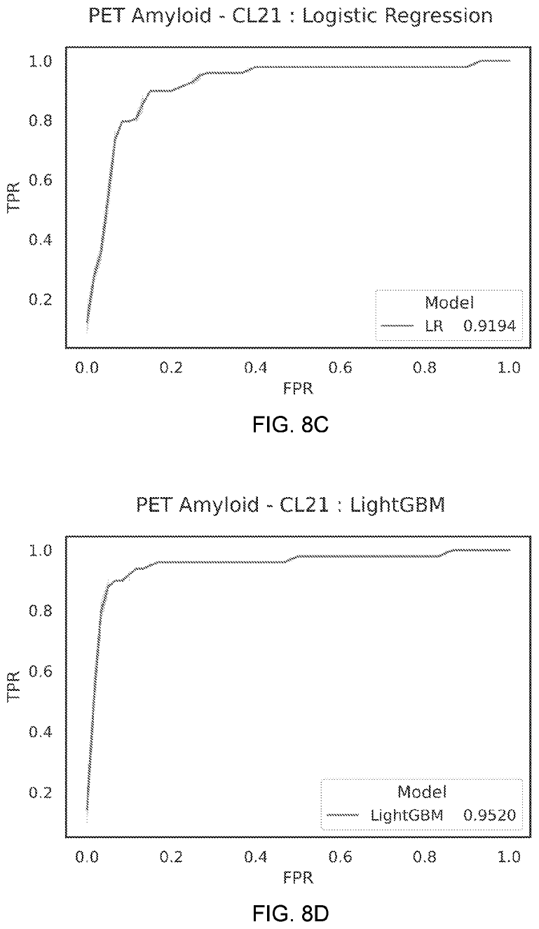

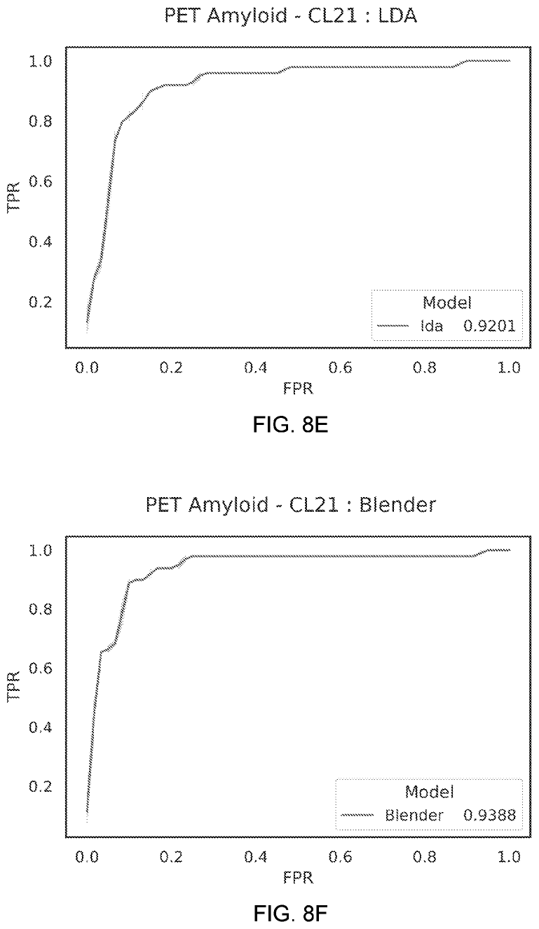

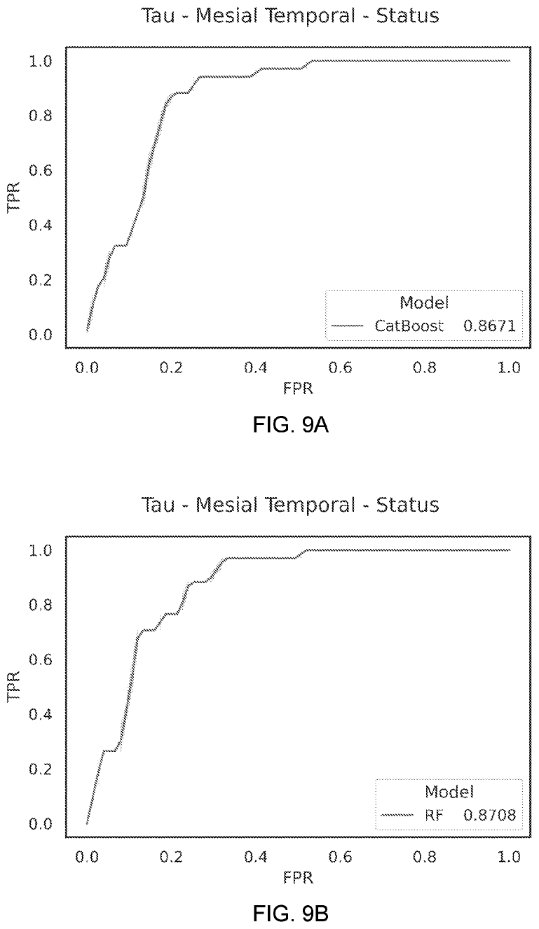

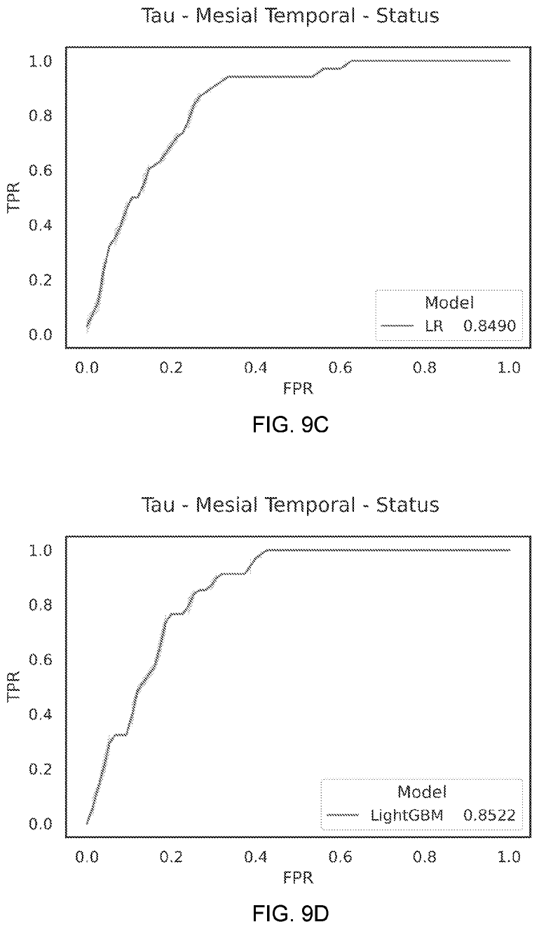

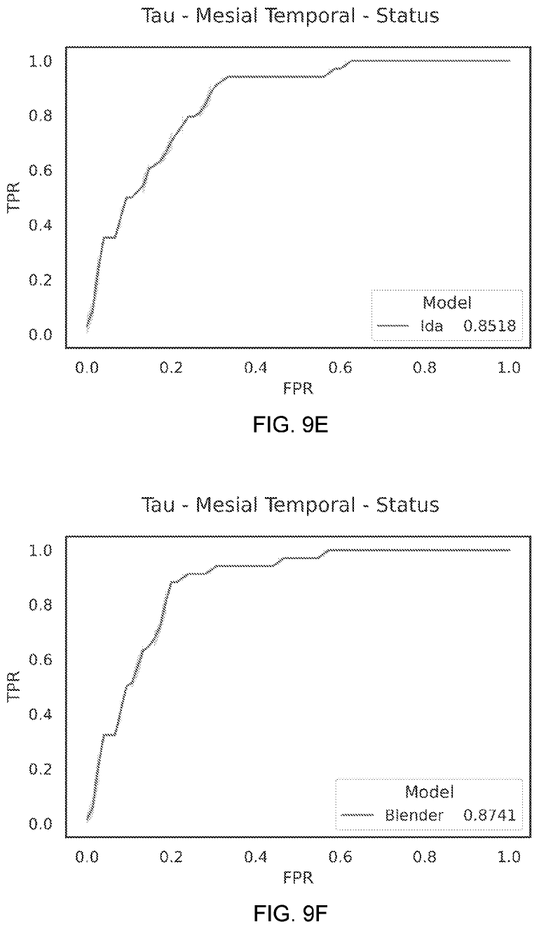

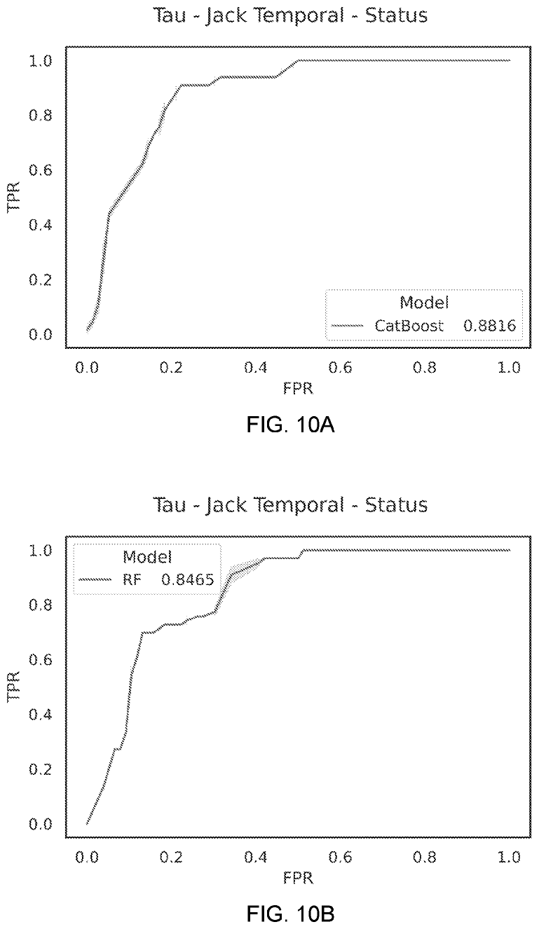

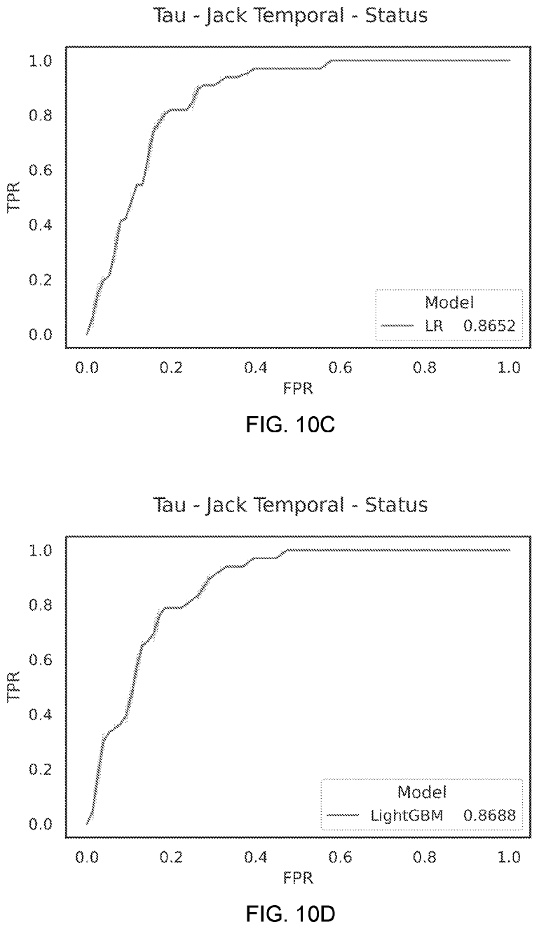

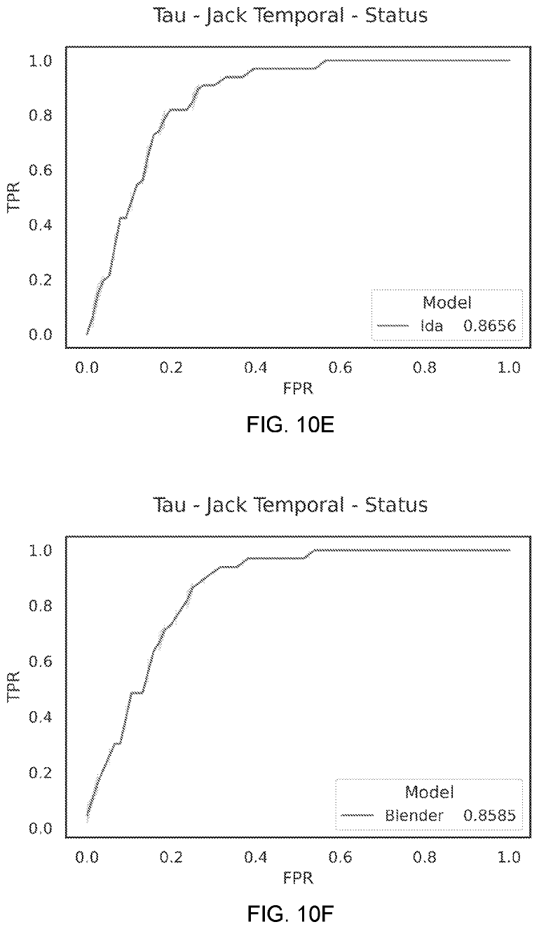

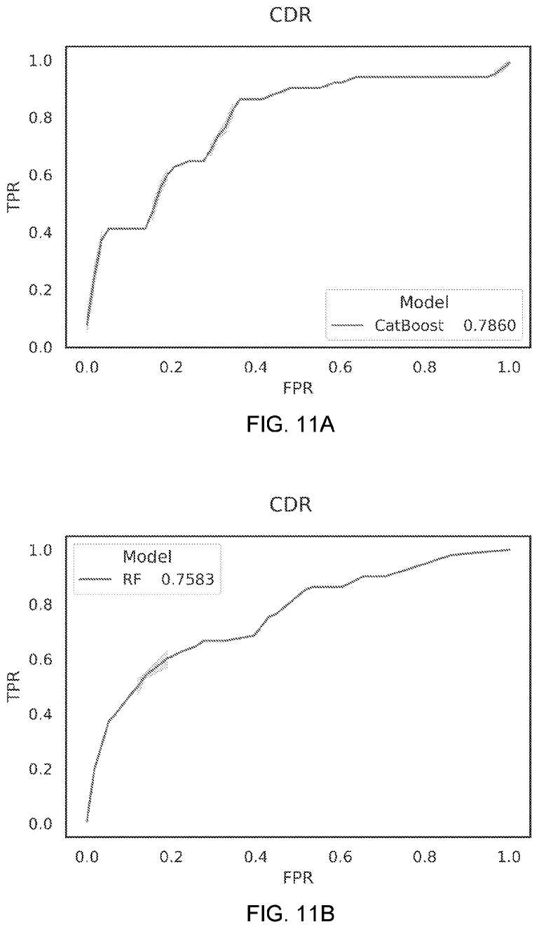

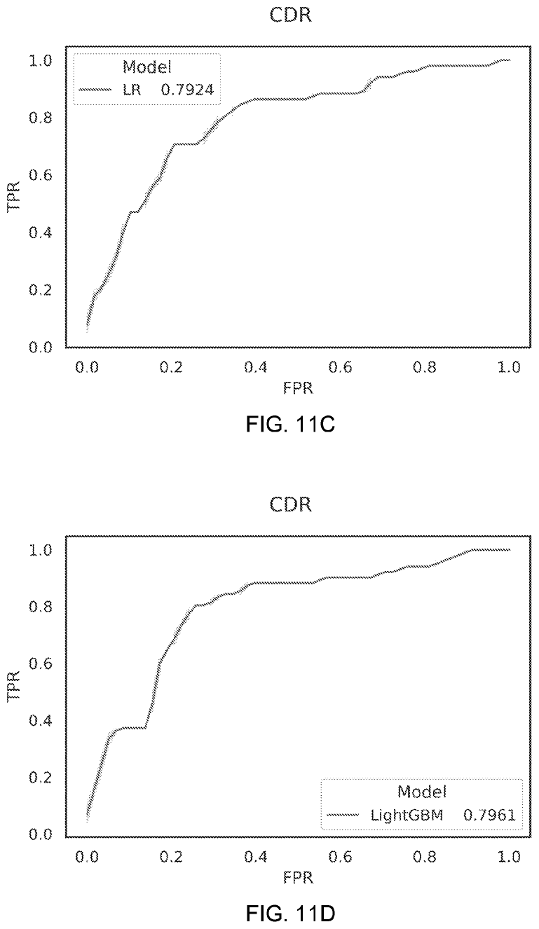

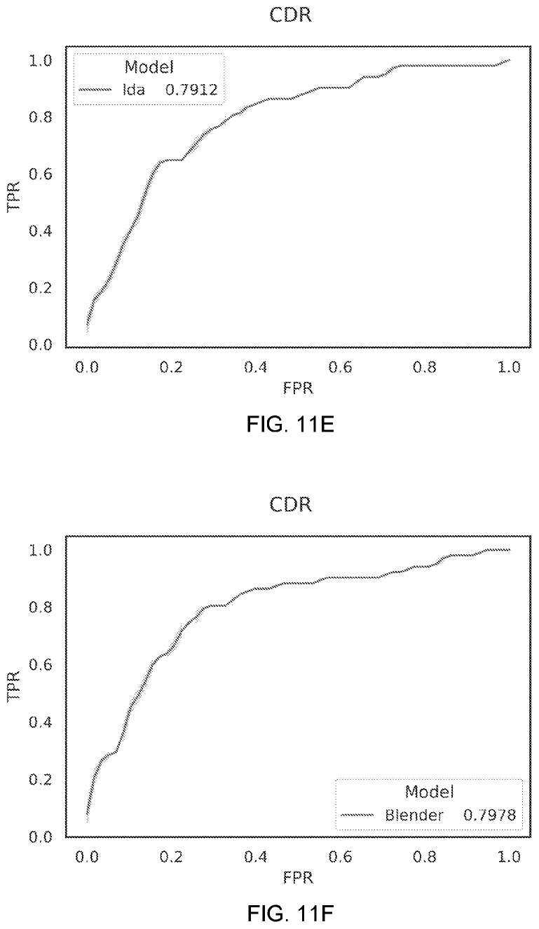

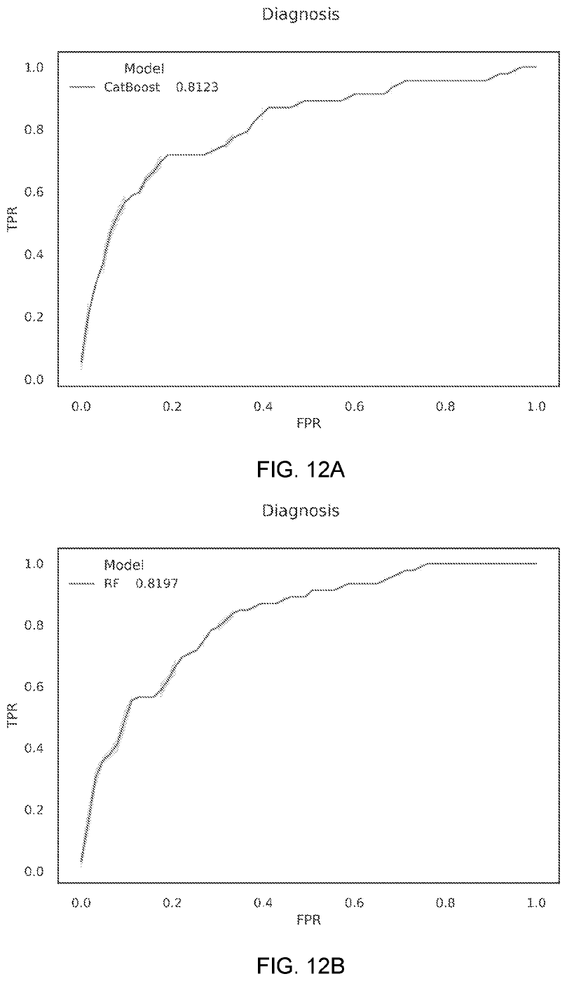

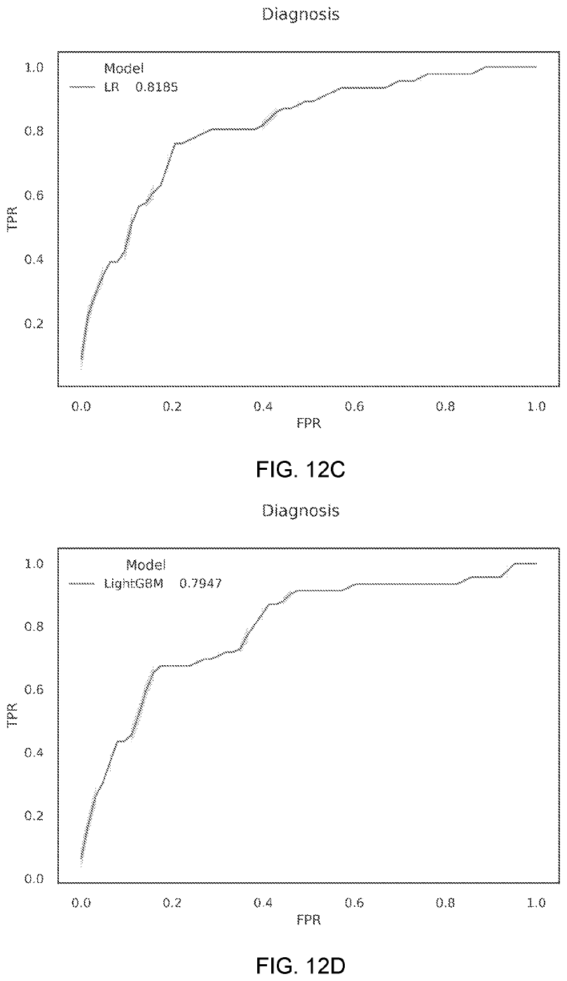

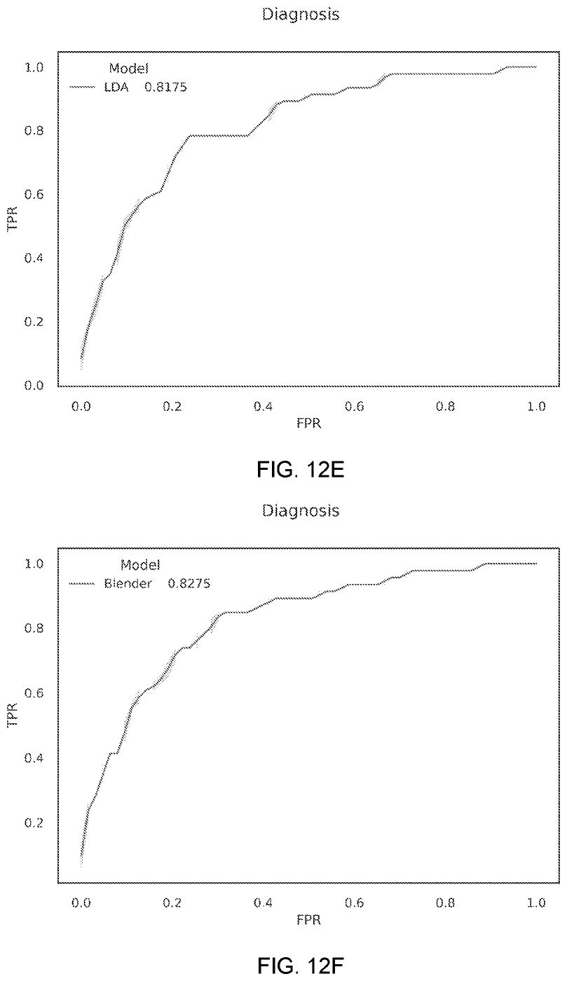

Receiver operating characteristic (ROC) curves for each surrogate variable with the test data are shown in FIGS. 7A-12F. The top performing model for each surrogate variable was selected based on which ROC curve most closely approached point (0,1). Feature importance plots for the top performing model for each surrogate variable are shown in FIGS. 13-18. Performance of the top performing trained models is summarized in Table 4.

TABLE 4

| | | | MOST |
|---|---|---|---|
| | | | INFLUENTIAL |
| | | TOP | PLASMA |
| SURROGATE | PERFORMANCE | PERFORMING | FEATURES |
| VARIABLE | (AUC) | MODEL | (TOP 5) |
| AMYLOID | 0.95 | Random | p181, AB42, |
| (CL 12) | | Forest | AB40, GFAP, |
| | | | sTREM2 |
| AMYLOID | 0.95 | CatBoost | p181, AB42, |
| (CL 21) | | | AB40, GFAP, |
| | | | sTREM2 |
| TAU (MT) | 0.86 | CatBoost | p181, GFAP, |
| | | | AB40, AB42, |
| | | | NFL |
| TAU (TJ) | 0.88 | CatBoost | p181, GFAP, |
| | | | AB42, AB40, |
| | | | TDP43 |
| CDR | 0.80 | LightGBM | GFAP, sTREM2, |
| | | | p181, AB42, |
| | | | AB40 |
| DIAGNOSIS | 0.82 | Logistic | GFAP, TDP43, |
| | | Regression | AB42, AB40, |
| | | | NFL |

As shown in Table 4, plasma Tau, Aβ-40, Aβ-42, GFAP, sTREM2, NFL, and TDP43 were generally the most influential plasma features in the models.

### 6.4. Example 4: Use of PREFER-AD of Example 3

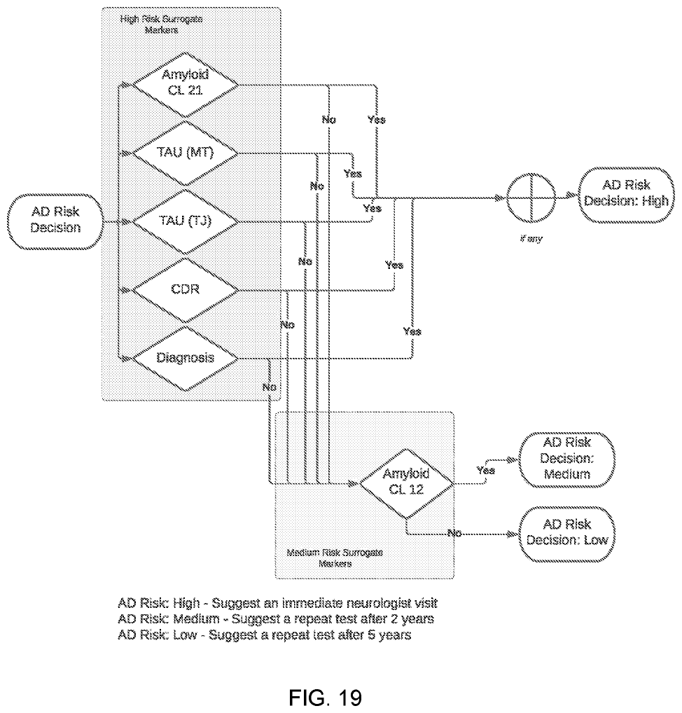

The AI-based algorithms described in Example 3 are used to assess a subject's risk for developing or already having AD from the subject's plasma markers. Age and gender is also used in the analysis. Individual AD risk scores for each surrogate variable described in Example 3 are generated using the algorithms described in Example 3, and the risk scores are used to classify the subject as high, medium, or low risk for AD according to the flow chart shown in FIG. 19.

In the flow chart, if one or more of the algorithms for the high risk surrogate variables (amyloid CL21, tau (MT), tau (TJ), CDR, diagnosis) predict that the subject is positive for one or more of the high risk surrogate variables, the subject is classified as having a high risk of having or developing AD. If none of the algorithms for the high risk variables predict that the subject is positive for a high risk variable, but the algorithm for the medium risk surrogate variable (amyloid CL12) predicts that the subject is positive for the medium risk surrogate variable, the subject is classified as medium risk. If none of the algorithms for the high risk variables predict that the subject is positive for a high risk variable and the algorithm for the medium risk surrogate variable does not predict that the subject is positive for the medium risk surrogate variable, the subject is classified as low risk. Different follow-up actions are recommended for subjects classified as high, medium, or low risk. A subject classified as high risk is referred for an immediate neurologist visit, while a repeat test after two years is recommended for a subject classified as medium risk, and a repeat test after five years is recommended for a subject classified as low risk.

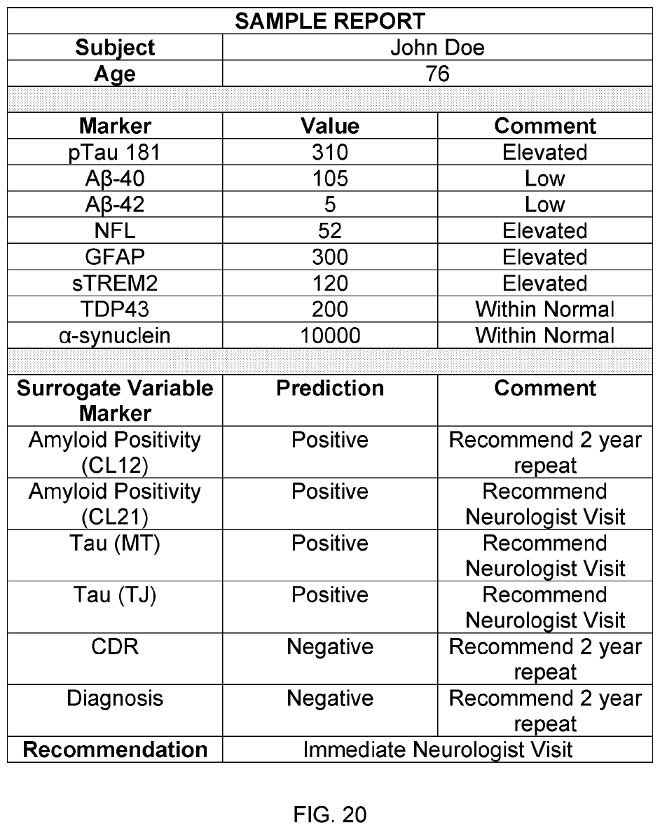

FIG. 20 shows a sample report for a 76 year old male subject. The sample report shows that the algorithms predict that the subject is positive for the amyloid (CL12 and CL21) and Tau (MT and TJ) AD surrogate variables, and negative for the CDR and diagnosis AD surrogate variables. As the subject is predicted to be positive for one or more of the high risk variables, the recommendation on the sample report is that the subject immediately visit a neurologist so that further testing for indicators of AD can be performed.

### 7. SPECIFIC EMBODIMENTS

### 7.1. Specific Embodiments: Group 1

Various aspects of the present disclosure are described in the embodiments set forth in the following numbered paragraphs, where reference to a previous numbered embodiment refers to a previous numbered embodiment in this Section 7.1.

1. A method for scoring a subject's risk for developing or already having Alzheimer's disease (AD), comprising:
(a) receiving a dataset associated with the subject, wherein said dataset comprises quantitative data for at least 4 protein markers in one or more fluid samples from the subject, optionally wherein:
   (i) the fluid samples are selected from blood and cerebral spinal fluid (CSF); and/or
   (ii) the protein markers comprise at least 3 of a tau peptide marker, an amyloid peptide marker, a neurodegeneration marker, a metabolic disorder marker, and an inflammation marker;
(b) generating an AD risk score from said dataset, thereby scoring the subject's risk for developing or already having AD.

2. The method of embodiment 1, wherein the dataset is obtained by a method comprising:
(a) obtaining said one or more fluid samples from the subject;
(b) performing an antibody or antigen assay on the one or more fluid samples to measure the levels of the at least 4 protein markers; and
(c) quantitating the at least 4 protein markers.

3. A method of analyzing a sample from a subject comprising the steps of:
(a) obtaining one or more fluid samples from a subject, optionally wherein the fluid samples are selected from blood and cerebral spinal fluid (CSF);
(b) performing an antibody or antigen assay on the one or more fluid samples to measure the levels of at least 4 protein markers, optionally wherein the protein markers comprise at least 3 of a tau peptide marker, an amyloid peptide marker, a neurodegeneration marker, a metabolic disorder marker, and an inflammation marker;
(c) generating quantitative values of the at least 4 protein markers;
(d) storing the quantitative values in a dataset associated with the subject; and
(e) generating an AD risk score from the dataset, thereby analyzing the sample from the subject.

4. The method of embodiment 3, which further comprises
(g) repeating steps (a) through (d) after at least 1 year;
(h) storing the quantitative values generated in step (g) in a subsequent dataset associated with the subject; and
(i) generating a subsequent AD risk score from the subsequent dataset.

5. A method of identifying a subject in need of AD testing, comprising:
(a) performing the method of embodiment 4 on fluid samples from a subject;
(b) determining if there is a change between the AD risk score and the subsequent AD risk score indicative of an increased risk for AD; and
(c) conducting further testing of the subject for indicators of AD.

6. The method of embodiment 5, wherein the further testing comprises PET amyloid and/or tau scans, amyloid scanning methods, lumbar puncture amyloid and/or tau procedures, structural MRI, neuropsychological testing or a combination thereof.

7. The method of embodiment 6, wherein the neuropsychological testing comprises one or more memory tests.

8. The method of embodiment 6 or embodiment 7, wherein the neuropsychological testing comprises conducting a cognitive test.

9. The method of embodiment 8, wherein the cognitive test is the Alzheimer's Initiative Preclinical Composite Cognitive test (“APCC”).

10. The method of any one of embodiments 1 to 9, wherein the step of generating an AD risk score method is computer implemented.

11. A computer implemented method for assessing a subject's risk for developing or already having AD, the method comprising executing, in a computer system having one or more processors coupled to a memory storing one or more computer readable instructions for execution by the one or more processors, the one or more computer readable instructions comprising instructions for:
(a) storing a dataset associated with the subject, wherein said dataset comprises quantitative data for at least 4 protein markers in one or more fluid samples from the subject, wherein:
   (i) the fluid samples are selected from blood and cerebral spinal fluid (CSF); and/or
   (ii) the protein markers comprise at least 3 of a tau peptide marker, an amyloid peptide marker, a neurodegeneration marker, a metabolic disorder marker, and an inflammation marker; and
(b) generating an AD risk score for the subject from the dataset, thereby scoring the subject's risk for developing or already having AD.

12. The method of embodiment 10 or embodiment 11, wherein the AD risk score is generated using a statistics- and/or artificial intelligence-based algorithm.

13. The method of embodiment 12, wherein the AD risk score is a generated using an artificial intelligence-based algorithm.

14. The method of embodiment 13, wherein the artificial intelligence-based algorithm is a logistic regression-based algorithm.

15. The method of embodiment 13, wherein the artificial intelligence-based algorithm is a light GBM-based algorithm.

16. The method of embodiment 13, wherein the artificial intelligence-based algorithm is a Random Forest-based algorithm.

17. The method of embodiment 13, wherein the artificial intelligence-based algorithm is a CatBoost-based algorithm.

18. The method of embodiment 13, wherein the artificial intelligence-based algorithm is a linear discriminant analysis-based algorithm.

19. The method of embodiment 13, wherein the artificial intelligence-based algorithm is an Adaptive Boosting-based algorithm.

20. The method of embodiment 13, wherein the artificial intelligence-based algorithm is an Extreme Gradient Boosting-based algorithm.

21. The method of embodiment 13, wherein the artificial intelligence-based algorithm is an Extra Trees-based algorithm.

22. The method of embodiment 13, wherein the artificial intelligence-based algorithm is a Naïve-Bayes-based algorithm.

23. The method of embodiment 13, wherein the artificial intelligence-based algorithm is a K-Nearest neighbor-based algorithm.

24. The method of embodiment 13, wherein the artificial intelligence-based algorithm is a Gradient Boosting-based algorithm.

25. The method of embodiment 13, wherein the artificial intelligence-based algorithm is a Support Vector-based algorithm.

26. The method of any one of embodiments 13 to 25, wherein the AD risk score predicts (i) the subject's brain amyloid load, (ii) the subject's brain tau load, (iii) brain neurodegeneration in the subject, or (iv) whether the subject exhibits symptoms sufficient for a diagnosis of mild cognitive impairment or AD.

27. The method of any one of embodiments 13 to 25, which comprises generating two or more AD risk scores from the dataset that individually predict (i) the subject's brain amyloid load, (ii) the subject's brain tau load, (iii) brain neurodegeneration in the subject, or (iv) whether the subject exhibits symptoms sufficient for a diagnosis of mild cognitive impairment or AD.

28. The method of any one of embodiments 13 to 25, which comprises generating three or more AD risk scores from the dataset that individually predict (i) the subject's brain amyloid load, (ii) the subject's brain tau load, (iii) brain neurodegeneration in the subject, or (iv) whether the subject exhibits symptoms sufficient for a diagnosis of mild cognitive impairment or AD.

29. The method of any one of embodiments 13 to 25, which comprises generating four or more AD risk scores from the dataset that individually predict (i) the subject's brain amyloid load, (ii) the subject's brain tau load, (iii) brain neurodegeneration in the subject, or (iv) whether the subject exhibits symptoms sufficient for a diagnosis of mild cognitive impairment or AD.

30. The method of any one of embodiments 13 to 25, which comprises generating five or more AD risk scores from the dataset that individually predict (i) the subject's brain amyloid load, (ii) the subject's brain tau load, (iii) brain neurodegeneration in the subject, or (iv) whether the subject exhibits symptoms sufficient for a diagnosis of mild cognitive impairment or AD.

31. The method of any one of embodiments 13 to 25, which comprises generating six or more AD risk scores from the dataset that individually predict (i) the subject's brain amyloid load, (ii) the subject's brain tau load, (iii) brain neurodegeneration in the subject, or (iv) whether the subject exhibits symptoms sufficient for a diagnosis of mild cognitive impairment or AD.

32. The method of any one of embodiments 26 to 31, which comprises generating an AD risk score from the dataset that predicts the subject's brain amyloid load.

33. The method of embodiment 32, which comprises generating an AD risk score from the dataset that predicts whether the subject is likely to have an amyloid PET centiloid value above or below a cutoff value, optionally wherein the AD risk score is a generated using a Random Forest-based algorithm or CatBoost-based algorithm.

34. The method of embodiment 32 or embodiment 33, which comprises generating an AD risk score from the dataset that predicts whether the subject is likely to have an amyloid PET centiloid value of less than 12, optionally wherein the AD risk score is a generated using a Random Forest-based algorithm.

35. The method of any one of embodiments 32 to 34, which comprises generating an AD risk score from the dataset that predicts whether the subject is likely to have an amyloid PET centiloid value of greater than or equal to 21, optionally wherein the AD risk score is a generated using a CatBoost-based algorithm.

36. The method of any one of embodiments 32 to 35, which comprises generating an AD risk score from the dataset that predicts whether the subject is likely to have a full brain amyloid standardized uptake value ratio (SUVR) above a cutoff value.

37. The method of any one of embodiments 32 to 36, which comprises generating an AD risk score from the dataset that predicts whether the subject is likely to have a volume of interest (VOI)-based amyloid standardized uptake value ratio (SUVR) or centiloid value above a cutoff value.

38. The method of any one of embodiments 26 to 37, which comprises generating an AD risk score from the dataset that predicts the subject's brain tau load.

39. The method of embodiment 38, which comprises generating an AD risk score from the dataset that predicts whether the subject is likely to have a tau load which is greater than a cutoff value, optionally wherein the cutoff value is based on a standardized measure of brain tau load.

40. The method of embodiment 38 or embodiment 39, which comprises generating an AD risk score from the dataset that predicts whether the subject is likely to have a Tau PET standardized uptake value ratio (SUVR) in the subject's mesial temporal region which is greater than a cutoff value, optionally wherein the AD risk score is a generated using a CatBoost-based algorithm.

41. The method of any one of embodiments 38 to 40, which comprises generating an AD risk score from the dataset that predicts whether the subject is likely to have a Tau PET standardized uptake value ratio (SUVR) in the subject's mesial temporal region which is greater than the 95^{th} percentile SUVR in healthy subjects, optionally wherein the AD risk score is a generated using a CatBoost-based algorithm.

42. The method of any one of embodiments 38 to 41, which comprises generating an AD risk score from the dataset that predicts whether the subject is likely to have a Tau PET standardized uptake value ratio (SUVR) in the subject's temporal region which is greater than a cutoff value, optionally wherein the AD risk score is a generated using a CatBoost-based algorithm.

43. The method of any one of embodiments 38 to 42, which comprises generating an AD risk score from the dataset that predicts whether the subject is likely to have a Tau PET standardized uptake value ratio (SUVR) in the subject's temporal region which is greater than the 95^{th} percentile SUVR in healthy subjects, optionally wherein the AD risk score is a generated using a CatBoost-based algorithm.

44. The method of any one of embodiments 40 to 43, wherein the Tau PET standardized uptake value ratio (SUVR) is a MK6240, Flortaucipir, RO948, Genentech Tau Probe (GTP) 1, or PI-2620 Tau PET standardized uptake value ratio (SUVR).

45. The method of embodiment 44, wherein the Tau PET standardized uptake value ratio (SUVR) is a MK6240 Tau PET standardized uptake value ratio (SUVR).

46. The method of any one of embodiments 38 to 45, which comprises generating an AD risk score from the dataset that predicts the subject's full brain tau load.

47. The method of any one of embodiments 38 to 46, which comprises generating an AD risk score from the dataset that predicts whether the subject is likely to have a volume of interest (VOI)-based tau standardized uptake value ratio (SUVR) above a cutoff value.

48. The method of any one of embodiments 26 to 47, which comprises generating an AD risk score from the dataset that predicts brain neurodegeneration in the subject.

49. The method of embodiment 48, which comprises generating an AD risk score from the dataset that predicts whether the subject is likely to have a clinical dementia rating indicative of brain neurodegeneration, optionally wherein the AD risk score is a generated using a Light GBM-based algorithm.

50. The method of embodiment 49, which comprises generating an AD risk score from the dataset that predicts whether the subject is likely to have a clinical dementia rating greater than or equal to 0.5, optionally wherein the AD risk score is a generated using a Light GBM-based algorithm.

51. The method of any one of embodiments 48 to 50, which comprises generating an AD risk score from the dataset that predicts whether the subject is likely to have a cognitive assessment test score indicative of brain neurodegeneration.

52. The method of embodiment 51, wherein the cognitive assessment is the mini-mental state examination (MMSE).

53. The method of embodiment 51, wherein the cognitive assessment is the Montreal Cognitive Assessment (MOCA).

54. The method of embodiment 51, wherein the cognitive assessment is the Disease Assessment Scale-Cognitive section (ADAS-Cog).

55. The method of embodiment 51, wherein the cognitive assessment is the Delis-Kaplan Executive Function System (D-KEFS) test.

56. The method of embodiment 51, wherein the cognitive assessment is the Addenbrookes Cognitive Assessment (ACE-R).

57. The method of any one of embodiments 48 to 56, which comprises generating an AD risk score from the dataset that predicts whether the subject is likely to have a physical measure of brain neurodegeneration.

58. The method of embodiment 57, wherein the physical measure is reduced cortical thickness indicative of brain neurodegeneration.

59. The method of embodiment 57, wherein the physical measure is loss of functional connectivity indicative of brain neurodegeneration.

60. The method of embodiment 57, wherein the physical measure is white matter hyperintensities indicative of brain neurodegeneration.

61. The method of any one of embodiments 26 to 60, which comprises generating an AD risk score from the dataset that predicts whether the subject is likely to have symptoms sufficient for a diagnosis of mild cognitive impairment or AD, optionally wherein the AD risk score is a generated using a CatBoost-based algorithm.

62. The method of any one of embodiments 26 to 61, which comprises generating (i) an AD risk score that predicts whether the subject is likely to have an amyloid PET centiloid value of less than a cutoff value, which is optionally 12; (ii) an AD risk score that predicts whether the subject is likely to have an amyloid PET centiloid value greater than or equal to a second cutoff value, which is optionally 21; (iii) an AD risk score that predicts whether the subject is likely to have a Tau PET standardized uptake value ratio (SUVR) in the subject's mesial temporal region which is greater than the 95^{th} percentile SUVR in healthy subjects; (iv) an AD risk score that predicts whether the subject is likely to have a Tau PET standardized uptake value ratio (SUVR) in the subject's temporal region which is greater than the 95^{th} percentile SUVR in healthy subjects; (v) an AD risk score that predicts whether the subject is likely to have a clinical dementia rating greater than or equal to 0.5; and (vi) an AD risk score that predicts whether the subject is likely to have symptoms sufficient for a diagnosis of mild cognitive impairment or AD.

63. The method of embodiment 62, which comprises generating an AD risk score that predicts whether the subject is likely to have an amyloid PET centiloid value of less than a cutoff value which is 12.

64. The method of embodiment 62 or embodiment 63, which comprises generating an AD risk score that predicts whether the subject is likely to have an amyloid PET centiloid value greater than or equal to a second cutoff value which is 21.

65. The method of any one of embodiments 10 to 64, which further comprises classifying the subject, based on the subject's AD risk score(s), into one of at least a first risk category and a second risk category, and optionally a third risk category.

66. The method of embodiment 65, wherein the first risk category indicates that the subject is at a low risk of developing AD.

67. The method of embodiment 66, which further comprises re-testing the subject for AD in approximately 1-5 years if the subject's AD risk score(s) indicate that the subject is at low risk of developing AD.

68. The method of embodiment 67, which further comprises re-testing the subject for AD in approximately 3-5 years if the subject's AD risk score(s) indicate that the subject is at low risk of developing AD.

69. The method of embodiment 66, which further comprises re-testing the subject for AD in approximately 1 year if the subject's AD risk score(s) indicate that the subject is at low risk of developing AD.

70. The method of any one of embodiments 65 to 69, wherein the second risk category indicates that the subject has AD or is at elevated risk of developing AD.

71. The method of embodiment 70, wherein the second risk category indicates that the subject has AD or is at high risk of developing AD.

72. The method of embodiment 71, which further comprises conducting further testing of the subject for indicators of AD if the subject has an AD risk score(s) indicating that the subject has AD or is at high risk of developing AD.

73. The method of embodiment 72, wherein the further testing comprises PET amyloid and/or tau scans, amyloid scanning methods, lumbar puncture amyloid and/or tau procedures, structural MRI, functional MRI, neuroinflammation scanning, diffuse tensor imaging, neuropsychological testing and/or a combination thereof.

74. The method of embodiment 73, wherein the neuropsychological testing comprises one or more memory tests.

75. The method of embodiment 73 or embodiment 74, wherein the neuropsychological testing comprises conducting a cognitive test.

76. The method of embodiment 75, wherein the cognitive test is the Alzheimer's Initiative Preclinical Composite Cognitive test (“APCC”).

77. The method of any one of embodiments 72 to 76, which further comprises generating, in a computerized system, a report recommending administering one or more AD therapeutics to the subject if the subject has an AD risk score(s) indicating that the subject has AD or is at high risk of developing AD.

78. The method of any one of embodiments 72 to 77, which further comprises administering one or more AD therapeutics to the subject if the subject has an AD risk score(s) indicating that the subject has AD or is at high risk of developing AD.

79. The method of embodiment 77 or embodiment 78, wherein the one or more AD therapeutics comprise an amyloid disease modifying therapy, a tau therapy, a cholinesterase inhibitor, an NMDA receptor blocker, or a combination thereof.

80. The method of embodiment 79, wherein the one or more AD therapeutics comprise aducanumab-avwa.

81. The method of any one of embodiments 72 to 80, which further comprises enrolling the subject in a clinical trial for a candidate AD therapeutic if the subject has an AD risk score(s) indicating that the subject has AD or is at high risk of developing AD.

82. The method of embodiment 81, which further comprises administering the candidate AD therapeutic to the subject.

83. The method of any one of embodiments 77 to 82, which comprises determining that the subject has AD.

84. The method of any one of embodiments 77 to 82, which comprises determining that the subject has a high risk of AD.

85. The method of any one of embodiments 65 to 84, which comprises classifying the subject, based on the subject's AD risk score(s), into one of at least a first risk category a second risk category, and a third risk category.

86. The method of embodiment 85, wherein the third risk category indicates that the subject is at moderate risk of developing AD.

87. The method of embodiment 86, which further comprises re-testing the subject for AD in approximately 1-2 years if the subject's AD risk score(s) indicate that the subject is at moderate risk of developing AD.

88. The method of any one of embodiments 63 to 87, further comprising generating, in a computerized system, a report comprising a representation of the risk category into which the has been classified.

89. The method of any one of embodiments 1 to 88, wherein the dataset further comprises the subject's family history of AD.

90. The method of any one of embodiments 1 to 89, wherein the dataset further comprises the age, gender or education of the subject, or any combination thereof.

91. The method of any one of embodiments 1 to 90, wherein the data set further comprises one or more genetic risk markers of AD.

92. The method of embodiment 91, wherein the one or more genetic risk markers of AD comprise APO E4, Clusterin (CLU), Sortilin-related receptor-1 (SORL1), ATP-binding cassette subfamily A member 7 (ABCA7), or a combination thereof.

93. The method of embodiment 92, wherein the genetic risk markers of AD comprise APO E4.

94. The method of any one of embodiments 1 to 92, wherein the data set does not comprise an APO E4 genetic risk marker of AD.

95. The method of any one of embodiments 1 to 94, wherein the dataset further comprises the results of a cognitive assessment.

96. The method of embodiment 95, wherein the cognitive assessment is the Alzheimer's Initiative Preclinical Composite Cognitive test (“APCC”).

97. The method of any one of embodiments 1 to 96, wherein the AD risk score(s) is/are provided as a percentage, multiplier value or absolute score.

98. The method of any one of embodiments 1 to 96, wherein when the AD risk score(s) provide a prediction (e.g., a prediction of whether the subject is likely to have a brain amyloid load above a cutoff value, a brain tau load above a cutoff level, neurodegeneration, symptoms sufficient for a diagnosis of mild cognitive impairment or AD, or a combination thereof), such AD risk score(s) is/are provided as a binary prediction (e.g., positive or negative).

99. The method of any one of embodiments 1 to 98 wherein the step of generating an AD risk score is computer implemented, and wherein the method further comprises providing a notification to the user recommending further testing when the subject has an AD risk score(s) indicative of a high risk for developing AD.

100. The method of any one of embodiments 1 to 99, wherein the step of generating an AD risk score is computer implemented, and wherein the method further comprises providing a notification to the user recommending a neurologic consultation when the subject has an AD risk score(s) indicative of a high risk for developing AD.

101. A method for monitoring the AD status of a subject with one or more AD risk factors, comprising:
(a) performing the method of any one of embodiments 1 to 100 on one or more fluid samples from the subject and assigning the subject a first AD risk score at a first time point;
(b) performing the method of any one of embodiments 1 to 100 on one or more fluid samples from the subject and assigning the subject a second AD risk score at a second time point;
(c) comparing the first AD risk score and the second AD risk score to determine if the subject's AD risk score has increased, thereby monitoring the AD status of the subject.

102. The method of embodiment 101, which further comprises:
(d) performing the method of any one of embodiments 1 to 100 on one or more fluid samples from the subject and assigning the subject a third AD risk score at a third time point;
(e) comparing the third AD risk score and the first AD risk score and/or second AD risk score to determine if the subject's AD risk score has increased and/or the rate of change of the subject's AD risk score, thereby continuing to monitor the AD status of the subject.

103. The method of embodiment 102, which further comprises:
(f) performing the method of any one of embodiments 1 to 100 on one or more fluid samples from the subject and assigning the subject a fourth AD risk score at a fourth time point;
(g) comparing the fourth AD risk score and the first AD risk score and/or second AD risk score and/or third AD risk score to determine if the subject's AD risk score has increased and/or the rate of change of the subject's AD risk score,
thereby continuing to monitor the AD status of the subject.

104. The method of any one of embodiments 1 to 103, wherein the protein markers comprise at least 5 protein markers.

105. The method of any one of embodiments 1 to 104, wherein the protein markers comprise one or more tau peptide markers.

106. The method of embodiment 105, wherein the one or more tau peptide markers comprise one or more phosphorylated tau peptide markers.

107. The method of embodiment 105 or embodiment 106, wherein the one or more tau peptide markers comprise p-tau 217.

108. The method of any one of embodiments 105 to 107, wherein the one or more tau peptide markers comprise p-tau 181.

109. The method of any one of embodiments 105 to 108, wherein the one or more tau peptide markers comprise p-tau 231.

110. The method of any one of embodiments 105 to 108, wherein the one or more tau peptide markers comprise p-tau 235.

111. The method of any one of embodiments 1 to 110, wherein the protein markers comprise one or more amyloid peptide markers.

112. The method of embodiment 111, wherein the one or more amyloid peptide markers comprise Aβ-40.

113. The method of embodiment 111 or embodiment 112, wherein the one or more amyloid peptide markers comprise Aβ-42.

114. The method of any one of embodiments 110 to 113, wherein the one or more amyloid peptide markers comprise the ratio of Aβ-40:Aβ-42.

115. The method of any one of embodiments 110 to 114, wherein the one or more amyloid peptide markers comprise the ratio of Aβ-42:Aβ-40.

116. The method of any one of embodiments 1 to 115, wherein the protein markers comprise one or more neurodegeneration markers.

117. The method of embodiment 116, wherein the one or more neurodegeneration markers comprise neurofilament light (“NFL”).

118. The method of embodiment 116 or embodiment 117, wherein the one or more neurodegeneration markers comprise glial fibrillary acidic protein (“GFAP”).

119. The method of any one of embodiments 1 to 118, wherein the protein markers comprise one or more metabolic disorder markers.

120. The method of embodiment 119, wherein the one or more metabolic disorder markers comprise HbA1c.

121. The method of any one of embodiments 1 to 120, wherein the protein markers comprise one or more inflammation markers.

122. The method of embodiment 121, wherein the one or more inflammation markers comprise C reactive protein (“CRP”).

123. The method of embodiment 121 or embodiment 122, wherein the one or more inflammation markers comprise interleukin-6 (“IL-6”).

124. The method of any one of embodiments 121 to 123, wherein the one or more inflammation markers comprise tumor necrosis factor (“TNF”).

125. The method of any one of embodiments 121 to 124, wherein the one or more inflammation markers comprise soluble TREM 2 (“sTREM-2”).

126. The method of any one of embodiments 121 to 125, wherein the one or more inflammation markers comprise a heat shock protein.

127. The method of any one of embodiments 121 to 126, wherein the one or more inflammation markers comprise YKL-40.

128. The method of any one of embodiments 1 to 127, wherein the protein markers further comprise one or more markers other than a tau peptide marker, an amyloid peptide marker, a neurodegeneration marker, a metabolic disease marker, and an inflammation marker (“other markers”), said other markers optionally comprising a proteinopathy marker, e.g., a frontotemporal lobe dementia (FTLD) marker, a Parkinson's Disease marker, a Lewy Body dementia marker, or a combination thereof.

129. The method of embodiment 128, wherein the one or more other markers comprise α-synuclein.

130. The method of embodiment 128 or embodiment 129, wherein the one or more other markers comprise TDP-43.

131. The method of any one of embodiments 1 to 130, wherein the one or more markers comprise one or more amyloid markers, one or more tau markers, and one or more neurodegeneration markers.

132. The method of embodiment 131, wherein the one or more amyloid markers comprise Aβ-40.

133. The method of embodiment 131 or embodiment 132, wherein the one or more amyloid markers comprise Aβ-42.

134. The method of any one of embodiments 131 to 133, wherein the one or more tau markers comprise p-tau 217, p-tau 181, p-tau 231, or p-tau 235.

135. The method of any one of embodiments 131 to 134, wherein the one or more tau markers comprise p-tau 217.

136. The method of any one of embodiments 131 to 134, wherein the one or more tau markers comprise p-tau 181.

137. The method of any one of embodiments 131 to 134, wherein the one or more tau markers comprise p-tau 231.

138. The method of any one of embodiments 131 to 134, wherein the one or more tau markers comprise p-tau 235.

139. The method of any one of embodiments 131 to 138, wherein the one or more neurodegeneration markers comprise NFL.

140. The method of any one of embodiments 131 to 139, wherein the one or more neurodegeneration markers comprise GFAP.

141. The method of any one of embodiments 131 to 140, wherein the one or more markers further comprise sTREM-2.

142. The method of any one of embodiments 131 to 141, wherein the one or more markers further comprise TDP-43.

143. The method of any one of embodiments 131 to 142, wherein the one or more markers further comprise α-synuclein.

144. The method of any one of embodiments 1 to 143, wherein the protein markers comprise at least 4 blood markers comprising at least 3 of a tau peptide marker, an amyloid peptide marker, a neurodegeneration marker, a metabolic disorder marker (which is optionally a diabetes marker), and an inflammation marker, optionally in combination with one or more CSF protein markers.

145. The method of any one of embodiments 1 to 143, wherein the protein markers comprise at least 5 blood markers comprising at least 3 of a tau peptide marker, an amyloid peptide marker, a neurodegeneration marker, a metabolic disorder marker (which is optionally a diabetes marker), and an inflammation marker, optionally in combination with one or more CSF protein markers.

146. The method of any one of embodiments 1 to 143, wherein the protein markers comprise at least 6 blood markers comprising at least 3 of a tau peptide marker, an amyloid peptide marker, a neurodegeneration marker, a metabolic disorder marker (which is optionally a diabetes marker), and an inflammation marker, optionally in combination with one or more CSF protein markers.

147. The method of any one of embodiments 1 to 143, wherein the protein markers comprise at least 7 blood markers comprising at least 3 of a tau peptide marker, an amyloid peptide marker, a neurodegeneration marker, a metabolic disorder marker (which is optionally a diabetes marker), and an inflammation marker, optionally in combination with one or more CSF protein markers.

148. The method of any one of embodiments 1 to 143, wherein the protein markers comprise at least 8 blood markers comprising at least 3 of a tau peptide marker, an amyloid peptide marker, a neurodegeneration marker, a metabolic disorder marker (which is optionally a diabetes marker), and an inflammation marker, optionally in combination with one or more CSF protein markers.

149. The method of any one of embodiments 1 to 143, wherein the protein markers comprise at least 9 blood markers comprising at least 3 of a tau peptide marker, an amyloid peptide marker, a neurodegeneration marker, a metabolic disorder marker (which is optionally a diabetes marker), and an inflammation marker, optionally in combination with one or more CSF protein markers.

150. The method of any one of embodiments 1 to 143, wherein the protein markers comprise at least 10 blood markers comprising at least 3 of a tau peptide marker, an amyloid peptide marker, a neurodegeneration marker, a metabolic disorder marker (which is optionally a diabetes marker), and an inflammation marker, optionally in combination with one or more CSF protein markers.

151. The method of any one of embodiments 1 to 150, wherein the fluid samples are blood samples.

152. The method of any one of embodiments 1 to 150, wherein the fluid samples are samples are a combination of blood samples and CSF samples.

153. The method of any one of embodiments 1 to 150, wherein the fluid samples are CSF samples.

154. The method of embodiment 151 or embodiment 152, wherein the blood samples are plasma samples.

155. The method of any one of embodiments 1 to 154, wherein the subject is 30-39 years of age.

156. The method of embodiment 155, which further comprises repeating steps (a) and (b) of embodiment 1 after one or more years.

157. The method of embodiment 156, wherein steps (a) and (b) of embodiment 1 are repeated on an annual, biennial, triennial, quadrennial or quinquennial basis.

158. The method of any one of embodiments 1 to 152, wherein the subject is 40-49 years of age.

159. The method of embodiment 158, which further comprises repeating steps (a) and (b) of embodiment 1 after one or more years.

160. The method of embodiment 159, wherein steps (a) and (b) of embodiment 1 are repeated on an annual, biennial, triennial, quadrennial or quinquennial basis.

161. The method of any one of embodiments 1 to 152, wherein the subject is 50-59 years of age.

162. The method of embodiment 161, which further comprises repeating steps (a) and (b) of embodiment 1 after one or more years.

163. The method of embodiment 162, wherein steps (a) and (b) of embodiment 1 are repeated on an annual, biennial, triennial, quadrennial or quinquennial basis.

164. The method of any one of embodiments 1 to 152, wherein the subject is 60-69 years of age.

165. The method of embodiment 164, which further comprises repeating steps (a) and (b) of embodiment 1 after one or more years.

166. The method of embodiment 165, wherein steps (a) and (b) of embodiment 1 are repeated on an annual, biennial, triennial, quadrennial or quinquennial basis.

167. The method of any one of embodiments 1 to 152, wherein the subject is 70-79 years of age.

168. The method of embodiment 167, which further comprises repeating steps (a) and (b) of embodiment 1 after one or more years.

169. The method of embodiment 168, wherein steps (a) and (b) of embodiment 1 are repeated on an annual, biennial, triennial, quadrennial or quinquennial basis.

170. A method of producing an artificial intelligence-based algorithm for generating an AD risk score for a subject, the method comprising executing, in a computer system having one or more processors coupled to a memory storing one or more computer readable instructions for execution by the one or more processors, the one or more computer readable instructions comprising instructions for:
(a) storing a dataset comprising a plurality of patient records, each patient record comprising quantitative data for at least 4 protein markers in one or more fluid samples from the patient and data for one or more AD surrogate variables for the patient, wherein:
   (i) the fluid samples are selected from blood and cerebral spinal fluid (CSF); and/or
   (ii) the protein markers comprise at least 3 of a tau peptide marker, an amyloid peptide marker, a neurodegeneration marker, a metabolic disorder marker, and an inflammation marker; and
(b) training a machine learning model with at least a portion of the patient records, wherein the quantitative data for the at least 4 protein markers are input variables and the data for the AD surrogate variable are output variables for the machine learning model, thereby providing an artificial intelligence-based algorithm for generating an AD risk score.

171. The method of embodiment 170, wherein the artificial intelligence-based algorithm weights the at least 4 protein markers differentially.

172. The method of embodiment 170 or embodiment 171, wherein the one or more AD surrogate variables comprise brain amyloid load.

173. The method of embodiment 172, wherein the patient data for brain amyloid load comprise standardized brain amyloid load data (e.g., PET centiloid data, PET SUVR data, or AmyloidIQ data).

174. The method of embodiment 172, wherein the patient data for brain amyloid load comprise amyloid PET centiloid data.

175. The method of any one of embodiments 172 to 174, wherein the at least 4 protein markers comprise one or more tau peptide markers, one or more amyloid peptide markers, one or more neurodegeneration markers, and one or more neuroinflammation markers.

176. The method of embodiment 175, wherein the one or more tau peptide markers comprise one or more phosphorylated tau peptide markers (e.g., p-tau 181), the one or more amyloid peptide markers comprise Aβ-40, Aβ-42, Aβ-42:Aβ-40 ratio, Aβ-40:Aβ-42 ratio, or a combination thereof, the one or more neurodegeneration markers comprise GFAP, and the one or more neuroinflammation markers comprise sTREM-2.

177. The method of embodiment 175 or embodiment 176, wherein the artificial intelligence-based algorithm weights one or more tau peptide markers (e.g., p-tau 181) greater than one or more amyloid peptide markers (e.g., Aβ-42:Aβ-40 ratio), and wherein the artificial intelligence-based algorithm weights one or more amyloid peptide markers (e.g., Aβ-42:Aβ-40 ratio) greater than one or more neurodegeneration markers (e.g., GFAP) and one or more neuroinflammation markers (e.g., sTREM-2).

178. The method of any one of embodiments 170 to 177, wherein the one or more AD surrogate variables comprise brain tau load.

179. The method of embodiment 178, wherein the patient data for brain tau load comprise standardized brain tau load data (e.g., PET SUVR data or TauIQ data).

180. The method of embodiment 178, wherein the patient data for brain tau load comprise Tau PET SUVR data.

181. The method of embodiment 179 or 180, wherein the Tau PET SUVR data comprise Tau PET SUVR data for the mesial temporal region of the brain.

182. The method of embodiment 180 or embodiment 181, wherein the Tau PET SUVR data comprise Tau PET SUVR data for the temporal region of the brain.

183. The method of any one of embodiments 180 to 182, wherein the Tau SUVR data is a MK6240, Flortaucipir, RO948, Genentech Tau Probe (GTP) 1, or PI-2620 Tau PET SUVR data.

184. The method of embodiment 183, wherein the Tau PET SUVR data is MK6240 Tau PET SUVR data.

185. The method of any one of embodiments 178 to 184, wherein the at least 4 protein markers comprise one or more tau peptide markers, one or more amyloid peptide markers, one or more neurodegeneration markers and, optionally, one or more proteinopathy markers.

186. The method of embodiment 185, wherein the one or more tau peptide markers comprise one or more phosphorylated tau peptide markers (e.g., p-tau 181), the one or more amyloid peptide markers comprise Aβ-40, Aβ-42, Aβ-42:Aβ-40 ratio, Aβ-40:Aβ-42 ratio, or a combination thereof, the one or more neurodegeneration markers comprise GFAP and/or NFL, and the one or more proteinopathy markers comprise TDP43.

187. The method of embodiment 185 or embodiment 186, wherein the artificial intelligence-based algorithm weights one or more of the one or more tau peptide markers (e.g., p-tau 181) greater than one or more neurodegeneration markers (e.g., GFAP), and wherein the artificial intelligence-based algorithm weights one or more neurodegeneration markers (e.g., GFAP) greater than one or more amyloid peptide markers (e.g., Aβ-42:Aβ-40 ratio).

188. The method of embodiment 185 or embodiment 186, wherein the artificial intelligence-based algorithm weights one or more tau peptide markers (e.g., p-tau 181) greater than one or more amyloid peptide markers (e.g., Aβ-42:Aβ-40 ratio), and wherein the artificial intelligence-based algorithm weights one or more amyloid peptide markers (e.g., Aβ-42:Aβ-40 ratio) greater than one or more neurodegeneration markers (e.g., GFAP).

189. The method of any one of embodiments 170 to 188, wherein the one or more AD surrogate variables comprise brain neurodegeneration.

190. The method of embodiment 189, wherein the patient data for brain neurodegeneration comprise clinical dementia rating data.

191. The method of embodiment 189 or embodiment 190, wherein the at least 4 protein markers comprise one or more tau peptide markers, one or more amyloid peptide markers, one or more neurodegeneration markers, and one or more inflammation markers.

192. The method of embodiment 191, wherein the one or more tau peptide markers comprise one or more phosphorylated tau peptide markers (e.g., p-tau 181), the one or more amyloid peptide markers comprise Aβ-40, Aβ-42, Aβ-42:Aβ-40 ratio, Aβ-40:Aβ-42 ratio, or a combination thereof, the one or more neurodegeneration markers comprise GFAP, and the one or more inflammation markers comprise sTREM-2.

193. The method of embodiment 191 or embodiment 192, wherein the artificial intelligence-based algorithm weights one or more tau peptide markers (e.g., p-tau 181) greater than one or more neurodegeneration markers (e.g., GFAP), and wherein the artificial intelligence-based algorithm weights one or more neurodegeneration markers (e.g., GFAP) greater than one or more amyloid markers (e.g., Aβ-42:Aβ-40 ratio) and one or more inflammation markers (e.g., sTREM-2).

194. The method of embodiment 191 or embodiment 192, wherein the artificial intelligence-based algorithm weights one or more neurodegeneration markers (e.g., GFAP) greater than one or more inflammation markers (e.g., sTREM-2), and wherein the artificial intelligence-based algorithm weights one or more inflammation markers (e.g., sTREM-2) greater than one or more tau peptide markers (e.g., p-tau 181) one or more amyloid peptide markers (e.g., Aβ-42:Aβ-40 ratio).

195. The method of any one of embodiments 170 to 194, wherein the one or more AD surrogate variables comprise clinical diagnosis of mild-cognitive impairment or AD.

196. The method of embodiment 195, wherein the patient data for clinical diagnosis of mild-cognitive impairment data comprise affirmative or negative diagnosis of mild-cognitive impairment or AD.

197. The method of embodiment 195 or embodiment 196, wherein the at least 4 protein markers comprise one or more tau peptide markers, one or more amyloid peptide markers, one or more neurodegeneration markers and, optionally, one or more proteinopathy markers.

198. The method of embodiment 185, wherein the one or more tau peptide markers comprise one or more phosphorylated tau peptide markers (e.g., p-tau 181), the one or more amyloid peptide markers comprise Aβ-40, Aβ-42, Aβ-42:Aβ-40 ratio, Aβ-40:Aβ-42 ratio, or a combination thereof, the one or more neurodegeneration markers comprise GFAP and/or NFL, and the one or more proteinopathy markers comprise TDP43.

199. The method of embodiment 197 or embodiment 198, wherein the artificial intelligence-based algorithm weights one or more tau peptide markers (e.g., p-tau 181) greater than one or more neurodegeneration markers (e.g., GFAP), and wherein the artificial intelligence-based algorithm weights one or more neurodegeneration markers (e.g., GFAP) greater than one or more amyloid peptide markers (e.g., Aβ-42:Aβ-40 ratio).

200. The method of embodiment 197 or embodiment 198, wherein the artificial intelligence-based algorithm weights one or more neurodegeneration markers (e.g., GFAP) greater than one or more proteinopathy markers (e.g., TDP43), and wherein the artificial intelligence-based algorithm weights one or more proteinopathy markers (e.g., TDP43) greater than one or more amyloid peptide markers (e.g., Aβ-42:Aβ-40 ratio) and greater than one or more inflammation markers (e.g., NFL).

201. The method of any one of embodiments 170 to 200, wherein the protein markers comprise the protein markers described in any one of embodiments 104 to 150.

202. The method of any one of embodiments 170 to 201, wherein each patient record further comprises the age of the patient.

203. The method of any one of embodiments 170 to 202, wherein each patient record further comprises the age of the patient at a tau PET scan.

204. The method of any one of embodiments 170 to 203, wherein each patient record further comprises the gender of the patient.

205. The method of any one of embodiments 170 to 204, wherein each patient record further comprises the education of the patient.

206. The method of any one of embodiments 170 to 205, wherein each patient record further comprises data for one or more genetic risk markers of AD.

207. The method of embodiment 206, wherein the one or more genetic risk markers of AD comprise APO E4, Clusterin (CLU), Sortilin-related receptor-1 (SORL1), ATP-binding cassette subfamily A member 7 (ABCA7), or a combination thereof.

208. The method of embodiment 207, wherein the genetic risk markers of AD comprise APO E4.

209. The method of any one of embodiments 170 to 207, wherein the patient records do not comprise an APO E4 genetic risk marker of AD.

210. The method of any one of embodiments 170 to 209, wherein the fluid samples are blood samples.

211. The method of any one of embodiments 170 to 209, wherein the fluid samples comprise a combination blood samples and CSF samples.

212. The method of any one of embodiments 170 to 209, wherein the fluid samples are CSF samples.

213. The method of embodiment 210 or embodiment 211, wherein the blood samples are plasma samples.

214. The method of any one of embodiments 170 to 213, which further comprises retraining the machine learning model with updated patient records.

215. The method of any one of embodiments 170 to 214, wherein the plurality of patient records comprises at least 100 patient records.

216. The method of any one of embodiments 170 to 214, wherein the plurality of patient records comprises at least 200 patient records.

217. The method of any one of embodiments 170 to 214, wherein the plurality of patient records comprises at least 300 patient records.

218. The method of any one of embodiments 170 to 214, wherein the plurality of patient records comprises at least 500 patient records.

219. The method of any one of embodiments 170 to 214, wherein the plurality of patient records comprises at least 1000 patient records.

220. The method of any one of embodiments 170 to 214, wherein the plurality of patient records comprises at least 5000 patient records.

221. The method of any one of embodiments 170 to 220, step (b) comprises training the machine learning model with at least 100 patient records.

222. The method of any one of embodiments 170 to 220, step (b) comprises training the machine learning model with at least 200 patient records.

223. The method of any one of embodiments 170 to 220, step (b) comprises training the machine learning model with at least 300 patient records.

224. The method of any one of embodiments 170 to 220, step (b) comprises training the machine learning model with at least 500 patient records.

225. The method of any one of embodiments 170 to 220, step (b) comprises training the machine learning model with at least 1000 patient records.

226. The method of any one of embodiments 170 to 220, step (b) comprises training the machine learning model with at least 5000 patient records.

227. The method of any one of embodiments 170 to 226, wherein step (b) further comprises testing the machine learning model with patient records not used to train the machine learning model.

228. The method of any one of embodiments 170 to 227, wherein the machine learning model is a logistic regression model.

229. The method of any one of embodiments 170 to 227, wherein the machine learning model is a light GBM model.

230. The method of any one of embodiments 170 to 227, wherein the machine learning model is a Random Forest model.

231. The method of any one of embodiments 170 to 227, wherein the machine learning model is a CatBoost model.

232. The method of any one of embodiments 170 to 227, wherein the machine learning model is a linear discriminant analysis model.

233. The method of any one of embodiments 170 to 227, wherein the machine learning model is an Adaptive Boosting model.

234. The method of any one of embodiments 170 to 227, wherein the machine learning model is an Extreme Gradient Boosting model.

235. The method of any one of embodiments 170 to 227, wherein the machine learning model is an Extra Trees model.

236. The method of any one of embodiments 170 to 227, wherein the machine learning model is a Naïve-Bayes model.

237. The method of any one of embodiments 170 to 227, wherein the machine learning model is a K-Nearest neighbor model.

238. The method of any one of embodiments 170 to 227, wherein the machine learning model is a Gradient Boosting model.

239. The method of any one of embodiments 170 to 227, wherein the machine learning model is a Support Vector model.

240. A method for scoring a subject's risk for developing or already having Alzheimer's disease (AD), comprising
(a) receiving a dataset associated with the subject, wherein said dataset comprises quantitative data for at least 4 protein markers in one or more fluid samples from the subject, optionally wherein:
   (i) the fluid samples are selected from blood and cerebral spinal fluid (CSF); and/or
   (ii) the protein markers comprise at least 3 of a tau peptide marker, an amyloid peptide marker, a neurodegeneration marker, a metabolic disorder marker, and an inflammation marker;
(b) generating an AD risk score from said dataset using an artificial intelligence-based algorithm produced by the method of any one of embodiments 170 to 239, thereby scoring the subject's risk for developing or already having AD.

241. The method of any one of embodiments 1 to 169, wherein the step of generating an AD risk score from the dataset comprises generating an AD risk score from said dataset using an artificial intelligence-based algorithm produced by the method of any one of embodiments 170 to 239.

242. A computer implemented method for assessing a subject's risk for developing or already having AD, the method comprising executing, in a computer system having one or more processors coupled to a memory storing one or more computer readable instructions for execution by the one or more processors, the one or more computer readable instructions comprising instructions for:
(a) storing a dataset associated with the subject, wherein said dataset comprises quantitative data for at least 4 protein markers in one or more fluid samples from the subject, wherein:
   (i) the fluid samples are selected from blood and cerebral spinal fluid (CSF); and/or
   (ii) the protein markers comprise at least 3 of a tau peptide marker, an amyloid peptide marker, a neurodegeneration marker, a metabolic disorder marker, and an inflammation marker; and
(b) generating an AD risk score for the subject from the dataset using the artificial intelligence-based algorithm produced by the method of any one of embodiments 170 to 239, thereby scoring the subject's risk for developing or already having AD.

243. A system configured to generate an AD risk score according to any one of the methods of any one of embodiments 10 to 169 and 240 to 242.

244. The system of embodiment 243, which comprises one or more processors coupled to a memory storing one or more computer readable instructions for execution by the one or more processors.

245. The system of embodiment 244, wherein the one or more computer readable instructions comprise instructions for:
(a) storing a dataset associated with the subject, wherein said dataset comprises quantitative data for at least 4 protein markers in one or more fluid samples from the subject, wherein:
   (i) the fluid samples are selected from blood and cerebral spinal fluid (CSF); and/or
   (ii) the protein markers comprise at least 3 of a tau peptide marker, an amyloid peptide marker, a neurodegeneration marker, a metabolic marker, and an inflammation marker; and
(b) generating an AD risk score for the subject from the dataset.

246. The system of embodiment 245, wherein the AD risk score is generated using a statistics- and/or artificial intelligence-based algorithm.

247. The system of embodiment 246, wherein the AD risk score is generated using the artificial intelligence-based algorithm produced by the method of any one of embodiments 170 to 239.

248. The system of any one of embodiments 244 to 247, wherein the computer readable instructions comprise instructions for generating a report for the subject.

249. The system of embodiment 248, wherein the report includes the subject's AD risk score(s).

250. The system of embodiment 248 or embodiment 249, wherein when the AD risk score(s) provide a prediction (e.g., a prediction of whether the subject is likely to have a brain amyloid load above a cutoff value, a brain tau load above a cutoff level, neurodegeneration, symptoms sufficient for a diagnosis of mild cognitive impairment or AD, or a combination thereof), such AD risk score(s) is/are provided in the report as a binary prediction (e.g., positive or negative).

251. The system of any one of embodiments 248 to 250, wherein the report includes one or more recommendations for the subject from the subject's AD risk score(s).

252. The system of any one of embodiments 248 to 251, wherein the computer readable instructions further comprise instructions for communicating the report to an external user.

253. The system of embodiment 252, wherein the external user is a medical practitioner (e.g., neurologist or primary care physician) or medical laboratory.

254. The system of any one of embodiments 248 to 253, wherein the computer readable instructions further comprise instructions for communicating the report to a data storage device.

255. The system of embodiment 254, wherein the data storage device is a local data storage device.

256. The system of embodiment 254, wherein the data storage device is a non-local data storage device (e.g., cloud storage).

257. The system of any one of embodiments 244 to 256, wherein the computer readable instructions further comprise instructions for classifying the subject's risk of having or developing AD.

258. The system of embodiment 257, wherein the instructions for classifying the subject's risk of having AD comprising instructions for classifying the subject into one of at least a first risk category and a second risk category, and optionally a third risk category for having or developing AD.

259. The system of embodiment 258, wherein first risk category is a low risk category, the second risk category is a high risk category and the third risk category is a medium (or moderate) risk category.

260. The system of any one of embodiments 257 to 259, when depending from any one of embodiments 248 to 255, wherein the report includes the subject's classification.

261. The system of embodiment 260, wherein the report includes a recommendation for the subject based on the subject's classification.

262. The system of embodiment 261, wherein the recommendation is a recommendation for further testing of the subject for indicators of AD if the subject is classified as having a high risk of having or developing AD.

263. The system of embodiment 261 or embodiment 262, wherein the recommendation is a recommendation for re-testing the subject for AD in approximately 1-5 years if the subject is classified as having a low risk of developing AD.

264. The system of embodiment 261 or embodiment 262, wherein the recommendation is a recommendation for re-testing the subject for AD in approximately 3-5 years if the subject is classified as having a low risk of developing AD.

265. The system of any one of embodiments 261 to 264, wherein the recommendation is a recommendation for re-testing the subject for AD in approximately 1-2 years if the subject is classified as having a medium risk of developing AD.

266. A system configured to produce an artificial intelligence-based algorithm for generating an AD risk score according to any one of embodiments 170 to 239.

267. The system of embodiment 266, which comprises one or more processors coupled to a memory storing one or more computer readable instructions for execution by the one or more processors.

268. The system of embodiment 267, wherein the one or more computer readable instructions comprise instructions for:
(a) storing a dataset comprising a plurality of patient records, each patient record comprising quantitative data for at least 4 protein markers in one or more fluid samples from the patient and data for one or more AD surrogate variables for the patient, wherein:
   (i) the fluid samples are selected from blood and cerebral spinal fluid (CSF); and/or
   (ii) the protein markers comprise at least 3 of a tau peptide marker, an amyloid peptide marker, a neurodegeneration marker, a metabolic disorder marker, and an inflammation marker; and
(b) training a machine learning model with at least a portion of the patient records, wherein the quantitative data for the at least 4 protein markers are input variables and the data for the AD surrogate variable are output variables for the machine learning model.

269. The system of any one of embodiments 267 to 268, wherein the computer readable instructions comprise instructions for a training mode and instructions for an AD risk score generating mode for scoring a subject's risk for developing or already having Alzheimer's disease (AD), wherein the training mode comprises instructions for producing an artificial intelligence-based algorithm for generating an AD risk score according to the method of any one of embodiments 170 to 239 and the AD risk score generating mode comprises instructions for scoring a subject's risk for developing or already having Alzheimer's disease (AD) according to the method of any one of embodiments 10 to 169 and 240 to 242.

270. The system of any one of embodiments 266 to 269, which comprises or further comprises the features of the system of any one of embodiments 243 to 265.

271. A system for generating an AD risk score for a subject, comprising one or more processors coupled to a memory storing one or more computer readable instructions for execution by the one or more processors, the one or more computer readable instructions comprising instructions for:
(a) storing a dataset comprising a plurality of patient records, each patient record comprising quantitative data for at least 4 protein markers in one or more fluid samples from the patient and data for one or more AD surrogate variables for the patient, wherein:
   (i) the fluid samples are selected from blood and cerebral spinal fluid (CSF); and/or
   (ii) the protein markers comprise at least 3 of a tau peptide marker, an amyloid peptide marker, a neurodegeneration marker, a metabolic disorder marker, and an inflammation marker;
(b) training a machine learning model with at least a portion of the patient records, wherein the quantitative data for the at least 4 protein markers are input variables and the data for the AD surrogate variable are output variables for the machine learning model to produce an AI-based algorithm for generating an AD risk score;
(c) generating an AD risk score for the subject using the AI-based algorithm;
(d) classifying the subject as having a low risk of developing AD if the AD risk score indicates that the subject is at a low risk of developing AD, having a medium (or moderate) risk of developing AD if the AD risk score indicates that the subject is at medium (or moderate) risk of developing AD, or high risk of having or developing AD if the AD risk score indicates that the subject is at high risk of having or developing AD; and
(e) generating a report comprising a representation of the risk category into which the has been classified and/or a recommendation for the subject based on the subject's classification.

272. A tangible, non-transitory computer-readable media comprising instructions executable by a processor for executing a method according to any one of embodiments 1 to 242.

273. A tangible, non-transitory computer-readable media comprising the computer readable instructions of any one of embodiments 243 to 265 and 267 to 271.

### 7.2. Specific Embodiments: Group 2

Various aspects of the present disclosure are described in the embodiments set forth in the following numbered paragraphs, where reference to a previous numbered embodiment refers to a previous numbered embodiment in this Section 7.2.

1. A method for scoring a subject's risk of developing or already having Alzheimer's Disease (“AD”), comprising:
(a) determining the levels of at least 4 or at least 5 protein markers in one or more fluid samples from the subject, optionally wherein:
   (i) the fluid samples are selected from blood, serum and cerebral spinal fluid (CSF); and/or
   (ii) the protein markers comprise at least 4 of a tau peptide marker, an amyloid peptide marker, a neurodegeneration marker, a metabolic disorder marker (which is optionally a diabetes marker), and an inflammation marker; and
(b) combining the levels of at least 4 or at least 5 protein markers to generate an AD risk score for the subject, thereby scoring the subject's risk of developing or already having AD.

2. The method of embodiment 1, which further comprises, prior to step (a), measuring the levels of the at least 4 or at least 5 protein markers.

3. The method of embodiment 2, wherein the levels are measured using antibody assays and/or antigen assays.

4. A method for scoring a subject's risk for developing or already having AD, comprising:
(a) receiving a dataset associated with the subject, wherein said dataset comprises quantitative data for at least 4 or at least 5 protein markers in one or more fluid samples from the subject, optionally wherein:
   (i) the fluid samples are selected from blood, serum and cerebral spinal fluid (CSF); and/or
   (ii) the protein markers comprise at least 4 of a tau peptide marker, an amyloid peptide marker, a neurodegeneration marker, a metabolic disorder marker (which is optionally a diabetes marker), and an inflammation marker;
(b) calculating an AD risk score from said dataset using, thereby scoring the subject's risk for developing or already having AD.

5. The method of embodiment 4, wherein the AD risk score is calculated using a statistics- and/or artificial intelligence-based algorithm.

6. The method of embodiment 4 or embodiment 5, wherein the dataset is obtained by a method comprising:
(a) obtaining said one or more fluid samples from the subject;
(b) performing an antibody or antigen assay on the one or more fluid samples to measure the levels of the at least 4 or at least 5 protein markers; and
(c) quantitating the at least 4 or at least 5 protein markers.

7. The method of any one of embodiments 1 to 6, wherein the risk score is a percentage, multiplier value or absolute score.

8. The method of any one of embodiments 1 to 7, wherein the protein markers comprise one or more tau peptide markers.

9. The method of embodiment 8, wherein the one or more tau peptide markers comprise p-tau 181.

10. The method of embodiment 8, wherein the one or more tau peptide markers comprise p-tau 217.

11. The method of any one of embodiments 1 to 10, wherein the protein markers comprise one or more amyloid peptide markers.

12. The method of embodiment 11, wherein the one or more amyloid peptide markers comprise Aβ-40.

13. The method of embodiment 11 or embodiment 12, wherein the one or more amyloid peptide markers comprise Aβ-42.

14. The method of any one of embodiments 11 to 13, wherein the one or more amyloid peptide markers comprise the ratio of Aβ-40:Aβ-42.

15. The method of any one of embodiments 1 to 14, wherein the protein markers comprise one or more neurodegeneration markers.

16. The method of embodiment 15, wherein the one or more neurodegeneration markers comprise neurofilament light (“NFL”).

17. The method of embodiment 15 or embodiment 16, wherein the one or more neurodegeneration markers comprise glial fibrillary acidic protein (“GFAP”).

18. The method of any one of embodiments 1 to 17, wherein the protein markers comprise one or more metabolic disorder markers.

19. The method of embodiment 18, wherein the one or more metabolic disorder markers comprise HbA1c.

20. The method of any one of embodiments 1 to 19, wherein the protein markers comprise one or more inflammation markers.

21. The method of embodiment 20, wherein the one or more inflammation markers comprise C reactive protein (“CRP”).

22. The method of embodiment 20 or embodiment 21, wherein the one or more inflammation markers comprise interleukin-6 (“IL-6”).

23. The method of any one of embodiments 20 to 22, wherein the one or more inflammation markers comprise tumor necrosis factor (“TNF”).

24. The method of any one of embodiments 20 to 23, wherein the one or more inflammation markers comprise soluble TREM 2 (“sTREM-2”).

25. The method of any one of embodiments 20 to 24, wherein the one or more inflammation markers comprise a heat shock protein.

26. The method of any one of embodiments 20 to 25, wherein the one or more inflammation markers comprise YKL-40.

27. The method of any one of embodiments 1 to 26, wherein the protein markers comprise one or more markers other than a tau peptide marker, an amyloid peptide marker, a neurodegeneration marker, a metabolic disease marker, and an inflammation marker (“other markers”), said other markers optionally comprising a frontotemporal lobe dementia (FTLD) marker, a Parkinson's Disease marker, a Lewy Body dementia marker, or a combination thereof.

28. The method of embodiment 27, wherein the one or more other markers comprise α-synuclein.

29. The method of embodiment 27 or embodiment 28, wherein the one or more other markers comprise TDP-43.

30. The method of any one of embodiments 1 to 29, wherein the levels of at least 2 or at least 3 of at least 4 or at least 5 protein markers are weighted equally.

31. The method of any one of embodiments 1 to 29, wherein the levels of at least 2 or at least 3 of at least 4 or at least 5 protein markers are weighted differentially.

32. The method of any one of embodiments 1 to 31, which further comprises binning the subject's AD risk score into one of at least a first risk category and a second risk category, and optionally a third risk category.

33. The method of embodiment 32, wherein the first risk category indicates that the subject is at a low risk of developing AD.

34. The method of embodiment 33, which further comprises re-testing the subject for AD in approximately 3-5 years.

35. The method of embodiment 32 or embodiment 33, wherein the second risk category indicates that the subject is at risk of developing AD.

36. The method of embodiment 35, wherein the second risk category indicates that the subject has AD or is at high risk of developing AD.

37. The method of embodiment 36, which further comprises conducting further testing of the subject for indicators of AD.

38. The method of embodiment 37, wherein the further testing comprises PET amyloid and/or tau scans, amyloid scanning methods, lumbar puncture amyloid and/or tau procedures, structural MRI, neuropsychological testing and/or a combination thereof.

39. The method of embodiment 38, wherein the neuropsychological testing comprises one or more memory tests.

40. The method of embodiment 38 or embodiment 39, wherein the neuropsychological testing comprises conducting a cognitive test.

41. The method of embodiment 40, wherein the cognitive test is the Alzheimer's Initiative Preclinical Composite Cognitive test (“APCC”).

42. The method of any one of embodiments 36 to 41, which comprises determining that the subject has AD and administering an AD therapeutic to the subject.

43. The method of embodiment 42, wherein the AD therapeutic is selected an amyloid disease modifying therapy, a tau therapy, a cholinesterase inhibitor, an NMDA receptor blocker, or a combination thereof.

44. The method of any one of embodiments 36 to 41, which comprises determining that the subject has AD and enrolling the subject in a clinical trial for a candidate AD therapeutic.

45. The method of embodiment 42, which further comprises administering the candidate AD therapeutic to the subject.

46. The method of any one of embodiments 32, 33, 35 and 36, which comprises binning the subject's AD risk score into a first risk category, a second risk category and a third risk category.

47. The method of embodiment 46, wherein the third risk category indicates that the subject is at moderate risk of developing AD.

48. The method of embodiment 47, which further comprises re-testing the subject for AD in approximately 1-2 years.

49. The method of any one of embodiments 1 to 48, wherein the risk score further comprises the subject's family history of AD.

50. The method of any one of embodiments 1 to 49, wherein the risk score further comprises the age, gender and education of the subject.

51. The method of an one of embodiments 1 to 50, wherein the risk score further comprises one or more genetic risk markers of AD.

52. The method of embodiment 51, wherein the one or more genetic risk markers of AD comprise APO E4, Clusterin (CLU), Sortilin-related receptor-1 (SORL1), ATP-binding cassette subfamily A member 7 (ABCA7).

53. The method of embodiment 52, wherein the genetic risk markers of AD comprise APO E4.

54. The method of any one of embodiments 1 to 53, wherein the risk score further comprises the results of a cognitive assessment.

55. The method of embodiment 54, wherein the cognitive assessment is the Alzheimer's Initiative Preclinical Composite Cognitive test (“APCC”).

56. The method of any one of embodiments 1 to 55, wherein the subject is 30-39 years of age.

57. The method of embodiment 56, which further comprises repeating steps (a) and (b) of embodiment 1 after one or more years.

58. The method of embodiment 57, wherein steps (a) and (b) of embodiment 1 are repeated on an annual, biennial, triennial, quadrennial or quinquennial basis.

59. The method of any one of embodiments 1 to 55, wherein the subject is 40-49 years of age.

60. The method of embodiment 59, which further comprises repeating steps (a) and (b) of embodiment 1 after one or more years.

61. The method of embodiment 60, wherein steps (a) and (b) of embodiment 1 are repeated on an annual, biennial, triennial, quadrennial or quinquennial basis.

62. The method of any one of embodiments 1 to 55, wherein the subject is 50-59 years of age.

63. The method of embodiment 62, which further comprises repeating steps (a) and (b) of embodiment 1 after one or more years.

64. The method of embodiment 63, wherein steps (a) and (b) of embodiment 1 are repeated on an annual, biennial, triennial, quadrennial or quinquennial basis.

65. The method of any one of embodiments 1 to 55, wherein the subject is 60-69 years of age.

66. The method of embodiment 65, which further comprises repeating steps (a) and (b) of embodiment 1 after one or more years.

67. The method of embodiment 66, wherein steps (a) and (b) of embodiment 1 are repeated on an annual, biennial, triennial, quadrennial or quinquennial basis.

68. The method of any one of embodiments 1 to 55, wherein the subject is 70-79 years of age.

69. The method of embodiment 68, which further comprises repeating steps (a) and (b) of embodiment 1 after one or more years.

70. The method of embodiment 69, wherein steps (a) and (b) of embodiment 1 are repeated on an annual, biennial, triennial, quadrennial or quinquennial basis.

71. A method of analyzing a sample from a subject comprising the steps of:
(a) obtaining one or more fluid samples from a subject, optionally wherein the fluid samples are selected from blood, serum and cerebral spinal fluid (CSF);
(b) performing an antibody or antigen assay on the one or more fluid samples to measure the levels of at least 4 or at least 5 protein markers, optionally wherein the protein markers comprise at least 4 of a tau peptide marker, an amyloid peptide marker, a neurodegeneration marker, a diabetes and/or metabolic marker, and an inflammation marker;
(c) generating quantitative values of the at least 4 or at least 5 protein markers;
(d) storing the quantitative values in an initial dataset associated with the subject; and
(e) scoring the sample with an initial AD risk score comprising the levels of the at least 4 or at least 5 protein markers, thereby analyzing the sample from the subject.

72. The method of embodiment 71, which further comprises
(a) repeating steps (a) through (d) after at least 1 year;
(b) storing the quantitative values generated in step (g) in a subsequent dataset associated with the subject; and
(c) scoring the sample with a subsequent AD risk score comprising the levels of the at least 4 or at least 5 protein markers.

73. A method of identifying a subject in need of AD testing, comprising:
(a) performing the method of embodiment 72 on fluid samples from a subject;
(b) determining if there is a change between the initial AD risk score and the subsequent AD risk score indicative of an increased risk for AD;
(c) conducting further testing of the subject for indicators of AD.

74. The method of embodiment 73, wherein the further testing comprises PET amyloid and/or tau scans, amyloid scanning methods, lumbar puncture amyloid and/or tau procedures, structural MRI, neuropsychological testing or a combination thereof.

75. The method of embodiment 74, wherein the neuropsychological testing comprises one or more memory tests.

76. The method of embodiment 74 or embodiment 75, wherein the neuropsychological testing comprises conducting a cognitive test.

77. The method of embodiment 76, wherein the cognitive test is the Alzheimer's Initiative Preclinical Composite Cognitive test (“APCC”).

78. A computer implemented method for assessing a subject's risk for developing or already having AD, comprising, in a computer system having one or more processors coupled to a memory storing one or more computer readable instructions for execution by the one or more processors, the one or more computer readable instructions comprising instructions for:
(a) storing a dataset comprising a plurality of patient records, each patient record comprising quantitative data for at least 4 or at least 5 protein markers in one or more fluid samples from the subject, wherein:
   (i) the fluid samples are selected from blood, serum and cerebral spinal fluid (CSF); and/or
   (ii) the protein markers comprise at least 4 of a tau peptide marker, an amyloid peptide marker, a neurodegeneration marker, a diabetes and/or metabolic marker, and an inflammation marker; and
(b) generating an AD risk score for the subject using a weighted scoring system for the at least 4 or at least 5 protein markers.

79. The computer implemented method of embodiment 78, wherein the AD risk score is generated using a statistics- and/or artificial intelligence-based algorithm.

80. The computer implemented method of embodiment 78 or embodiment 79, which further comprises binning the subject's AD risk score into one of at least a first risk category and a second risk category, and optionally a third risk category.

81. The computer implemented method of embodiment 79, wherein the first risk category indicates that the subject is at a low risk of developing AD.

82. The computer implemented method of embodiment 79 or embodiment 81, wherein the second risk category indicates that the subject is at risk of developing AD.

83. The computer implemented method of embodiment 82, wherein the second risk category indicates that the subject has AD or is at high risk of developing AD.

84. The computer implemented method of any one of embodiments 79 to 83, which further comprises binning the subject's AD risk score into a third risk category.

85. The computer implemented method of embodiment 84, wherein the third risk category indicates that the subject is at moderate risk of developing AD.

86. The computer implemented method of any one of embodiments 78 to 85, wherein the risk score and dataset further comprise the subject's family history of AD.

87. The computer implemented method of any one of embodiments 78 to 86, wherein the risk score and dataset further comprise further comprise the age, gender and education of the subject.

88. The computer implemented method of any one of embodiments 78 to 87, wherein the risk score and data set further comprise one or more genetic risk markers of AD.

89. The computer implemented method of embodiment 88, wherein the one or more genetic risk markers of AD comprise APO E4, Clusterin (CLU), Sortilin-related receptor-1 (SORL1), ATP-binding cassette subfamily A member 7 (ABCA7).

90. The computer implemented method of embodiment 89, wherein the genetic risk markers of AD comprise APO E4.

91. The computer implemented method of any one of embodiments 78 to 90, wherein the risk score and dataset further comprise the results of a cognitive assessment.

92. The computer implemented method of embodiment 91, wherein the cognitive assessment is the Alzheimer's Initiative Preclinical Composite Cognitive test (“APCC”).

93. The computer implemented method of any one of embodiments 78 to 92, wherein the risk score is provided as a percentage, multiplier value or absolute score.

94. The computer implemented method of any one of embodiments 78 to 93, further comprising providing a notification to the user recommending further testing when the subject's risk score is indicative of a high risk for developing AD.

95. The method of any one of embodiments 1 to 77 or the computer implemented method of any one of embodiments 78 to 94, wherein the protein markers comprise at least 4 blood or serum markers, said blood or serum markers comprising at least 4 of a tau peptide marker, an amyloid peptide marker, a neurodegeneration marker, a metabolic disorder marker (which is optionally a diabetes marker), and an inflammation marker, optionally in combination with one or more CSF protein markers.

96. The method of any one of embodiments 1 to 77 or the computer implemented method of any one of embodiments 78 to 94, wherein the protein markers comprise at least 5 blood or serum markers, said blood or serum markers comprising at least 4 of a tau peptide marker, an amyloid peptide marker, a neurodegeneration marker, a metabolic disorder marker (which is optionally a diabetes marker), and an inflammation marker, optionally in combination with one or more CSF protein markers.

97. The method of any one of embodiments 1 to 77 or the computer implemented method of any one of embodiments 78 to 94, wherein the protein markers comprise at least 6 blood or serum markers, said blood or serum markers comprising at least 4 of a tau peptide marker, an amyloid peptide marker, a neurodegeneration marker, a metabolic disorder marker (which is optionally a diabetes marker), and an inflammation marker, optionally in combination with one or more CSF protein markers.

98. The method of any one of embodiments 1 to 77 or the computer implemented method of any one of embodiments 78 to 94, wherein the protein markers comprise at least 7 blood or serum markers, said blood or serum markers comprising at least 4 of a tau peptide marker, an amyloid peptide marker, a neurodegeneration marker, a metabolic disorder marker (which is optionally a diabetes marker), and an inflammation marker, optionally in combination with one or more CSF protein markers.

99. The method of any one of embodiments 1 to 77 or the computer implemented method of any one of embodiments 78 to 94, wherein the protein markers comprise at least 8 blood or serum markers, said blood or serum markers comprising at least 4 of a tau peptide marker, an amyloid peptide marker, a neurodegeneration marker, a metabolic disorder marker (which is optionally a diabetes marker), and an inflammation marker, optionally in combination with one or more CSF protein markers.

100. The method of any one of embodiments 1 to 77 or the computer implemented method of any one of embodiments 78 to 94, wherein the protein markers comprise at least 9 blood or serum markers, said blood or serum markers comprising at least 4 of a tau peptide marker, an amyloid peptide marker, a neurodegeneration marker, a metabolic disorder marker (which is optionally a diabetes marker), and an inflammation marker, optionally in combination with one or more CSF protein markers.

101. The method of any one of embodiments 1 to 77 or the computer implemented method of any one of embodiments 78 to 94, wherein the protein markers comprise at least 10 blood or serum markers, said blood or serum markers comprising at least 4 of a tau peptide marker, an amyloid peptide marker, a neurodegeneration marker, a metabolic disorder marker (which is optionally a diabetes marker), and an inflammation marker, optionally in combination with one or more CSF protein markers.

102. A method for monitoring the AD status of a subject with one or more AD risk factors, comprising:
(a) performing the method of any one of embodiments 1 to 101 on one or more fluid samples from the subject and assigning the subject a first AD risk score at a first time point;
(b) performing the method of any one of embodiments 1 to 101 on one or more fluid samples from the subject and assigning the subject a second AD risk score at a second time point;
(c) comparing the first AD risk score and the second AD risk score to determine if the subject's AD risk score has increased, thereby monitoring the AD status of the subject.

103. The method of embodiment 102, which further comprises:
(a) performing the method of any one of embodiments 1 to 101 on one or more fluid samples from the subject and assigning the subject a third AD risk score at a third time point;
(b) comparing the third AD risk score and the first AD risk score and/or second AD risk score to determine if the subject's AD risk score has increased and/or the rate of change of the subject's AD risk score, thereby continuing to monitor the AD status of the subject.

104. The method of embodiment 103, which further comprises:
(a) performing the method of any one of embodiments 1 to 101 on one or more fluid samples from the subject and assigning the subject a fourth AD risk score at a fourth time point;
(b) comparing the fourth AD risk score and the first AD risk score and/or second AD risk score and/or third AD risk score to determine if the subject's AD risk score has increased and/or the rate of change of the subject's AD risk score, thereby continuing to monitor the AD status of the subject.

105. A system configured to generate an AD risk score according to any one of the computer implemented methods of any one of embodiments 78 to 94.

106. The system of embodiment 105, which comprises one or more processors coupled to a memory storing one or more computer readable instructions for execution by the one or more processors.

107. The system of embodiment 106, wherein the one or more computer readable instructions comprise instructions for:
(a) storing a dataset comprising a plurality of patient records, each patient record comprising quantitative data for at least 4 or at least 5 protein markers in one or more fluid samples from the subject, wherein:
   (i) the fluid samples are selected from blood, serum and cerebral spinal fluid (CSF); and/or
   (ii) the protein markers comprise at least 4 of a tau peptide marker, an amyloid peptide marker, a neurodegeneration marker, a diabetes and/or metabolic marker, and an inflammation marker; and
(b) generating an AD risk score for the subject using a weighted scoring system for the at least 4 or at least 5 protein markers.

108. The system of embodiment 107, wherein the AD risk score is generated using a statistics- and/or artificial intelligence-based algorithm.

### 8. CITATION OF REFERENCES

While various specific embodiments have been illustrated and described, it will be appreciated that various changes can be made without departing from the spirit and scope of the disclosure(s).

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A method for scoring a subject's risk for developing or already having Alzheimer's disease (AD), comprising:
(a) receiving a dataset associated with the subject, wherein said dataset comprises quantitative data for at least 4 protein markers in one or more fluid samples from the subject, optionally wherein:
(i) the fluid samples are selected from blood and cerebral spinal fluid (CSF); and/or
(ii) the protein markers comprise at least 3 of a tau peptide marker, an amyloid peptide marker, a neurodegeneration marker, a metabolic disorder marker, and an inflammation marker;
(b) generating an AD risk score from said dataset, thereby scoring the subject's risk for developing or already having AD.

2. The method of claim 1, wherein the dataset is obtained by a method comprising:
(a) obtaining said one or more fluid samples from the subject;
(b) performing an antibody or antigen assay on the one or more fluid samples to measure the levels of the at least 4 protein markers; and
(c) quantitating the at least 4 protein markers.

3. A method of analyzing a sample from a subject comprising the steps of:
(a) obtaining one or more fluid samples from a subject, optionally wherein the fluid samples are selected from blood and cerebral spinal fluid (CSF);
(b) performing an antibody or antigen assay on the one or more fluid samples to measure the levels of at least 4 protein markers, optionally wherein the protein markers comprise at least 3 of a tau peptide marker, an amyloid peptide marker, a neurodegeneration marker, a metabolic disorder marker, and an inflammation marker;
(c) generating quantitative values of the at least 4 protein markers;
(d) storing the quantitative values in a dataset associated with the subject; and
(e) generating an AD risk score from the dataset, thereby analyzing the sample from the subject.

4. The method of claim 3, which further comprises
(a) repeating steps (a) through (d) after at least 1 year;
(b) storing the quantitative values generated in step (g) in a subsequent dataset associated with the subject; and
(c) generating a subsequent AD risk score from the subsequent dataset.

5. A method of identifying a subject in need of AD testing, comprising:
(a) performing the method of claim 4 on fluid samples from a subject;
(b) determining if there is a change between the AD risk score and the subsequent AD risk score indicative of an increased risk for AD; and
(c) conducting further testing of the subject for indicators of AD.

6. The method of claim 5, wherein the further testing comprises PET amyloid and/or tau scans, amyloid scanning methods, lumbar puncture amyloid and/or tau procedures, structural MRI, neuropsychological testing or a combination thereof.

7. The method of any one of claims 1 to 6, wherein the step of generating an AD risk score method is computer implemented.

8. A computer implemented method for assessing a subject's risk for developing or already having AD, the method comprising executing, in a computer system having one or more processors coupled to a memory storing one or more computer readable instructions for execution by the one or more processors, the one or more computer readable instructions comprising instructions for:
(a) storing a dataset associated with the subject, wherein said dataset comprises quantitative data for at least 4 protein markers in one or more fluid samples from the subject, wherein:
(i) the fluid samples are selected from blood and cerebral spinal fluid (CSF); and/or
(ii) the protein markers comprise at least 3 of a tau peptide marker, an amyloid peptide marker, a neurodegeneration marker, a metabolic disorder marker, and an inflammation marker; and
(b) generating an AD risk score for the subject from the dataset, thereby scoring the subject's risk for developing or already having AD.

9. The method of claim 7 or claim 8, wherein the AD risk score is generated using a statistics- and/or artificial intelligence-based algorithm.

10. The method of claim 9, wherein the AD risk score is a generated using an artificial intelligence-based algorithm, optionally wherein the artificial intelligence-based algorithm is a logistic regression-based algorithm, a light GBM-based algorithm, a Random Forest-based algorithm, a CatBoost-based algorithm, a linear discriminant analysis-based algorithm, an Adaptive Boosting-based algorithm, an Extreme Gradient Boosting-based algorithm, an Extra Trees-based algorithm, a Naïve-Bayes-based algorithm, a K-Nearest neighbor-based algorithm, a Gradient Boosting-based algorithm, or a Support Vector-based algorithm.

11. The method of claim 10, wherein the AD risk score predicts (i) the subject's brain amyloid load, (ii) the subject's brain tau load, (iii) brain neurodegeneration in the subject, or (iv) whether the subject exhibits symptoms sufficient for a diagnosis of mild cognitive impairment or AD.

12. The method of claim 10 or claim 11, which comprises generating two or more, three or more, four or more, five or more, or six or more AD risk scores from the dataset that individually predict (i) the subject's brain amyloid load, (ii) the subject's brain tau load, (iii) brain neurodegeneration in the subject, or (iv) whether the subject exhibits symptoms sufficient for a diagnosis of mild cognitive impairment or AD.

13. The method of claim 11 or claim 12, which comprises generating an AD risk score from the dataset that predicts the subject's brain amyloid load.

14. The method of claim 13, which comprises generating an AD risk score from the dataset that predicts whether the subject is likely to have an amyloid PET centiloid value above or below a cutoff value, optionally wherein the AD risk score is a generated using a Random Forest-based algorithm or CatBoost-based algorithm.

15. The method of claim 13 or claim 14, which comprises generating an AD risk score from the dataset that predicts whether the subject is likely to have an amyloid PET centiloid value of less than 12, optionally wherein the AD risk score is a generated using a Random Forest-based algorithm.

16. The method of any one of claims 13 to 15, which comprises generating an AD risk score from the dataset that predicts whether the subject is likely to have an amyloid PET centiloid value of greater than or equal to 21, optionally wherein the AD risk score is a generated using a CatBoost-based algorithm.

17. The method of any one of claims 13 to 16, which comprises generating an AD risk score from the dataset that predicts whether the subject is likely to have a full brain amyloid standardized uptake value ratio (SUVR) above a cutoff value.

18. The method of any one of claims 13 to 17, which comprises generating an AD risk score from the dataset that predicts whether the subject is likely to have a volume of interest (VOI)-based amyloid standardized uptake value ratio (SUVR) or centiloid value above a cutoff value.

19. The method of any one of claims 11 to 18, which comprises generating an AD risk score from the dataset that predicts the subject's brain tau load.

20. The method of claim 19, which comprises generating an AD risk score from the dataset that predicts whether the subject is likely to have a tau load which is greater than a cutoff value, optionally wherein the cutoff value is based on a standardized measure of brain tau load.

21. The method of claim 19 or claim 20, which comprises generating an AD risk score from the dataset that predicts whether the subject is likely to have a Tau PET standardized uptake value ratio (SUVR) in the subject's mesial temporal region which is greater than a cutoff value, optionally wherein the AD risk score is a generated using a CatBoost-based algorithm.

22. The method of any one of claims 19 to 21, which comprises generating an AD risk score from the dataset that predicts whether the subject is likely to have a Tau PET standardized uptake value ratio (SUVR) in the subject's mesial temporal region which is greater than the 95^{th} percentile SUVR in healthy subjects, optionally wherein the AD risk score is a generated using a CatBoost-based algorithm.

23. The method of any one of claims 19 to 22, which comprises generating an AD risk score from the dataset that predicts whether the subject is likely to have a Tau PET standardized uptake value ratio (SUVR) in the subject's temporal region which is greater than a cutoff value, optionally wherein the AD risk score is a generated using a CatBoost-based algorithm.

24. The method of any one of claims 19 to 23, which comprises generating an AD risk score from the dataset that predicts whether the subject is likely to have a Tau PET standardized uptake value ratio (SUVR) in the subject's temporal region which is greater than the 95^{th} percentile SUVR in healthy subjects, optionally wherein the AD risk score is a generated using a CatBoost-based algorithm.

25. The method of any one of claims 21 to 24, wherein the Tau PET standardized uptake value ratio (SUVR) is a MK6240, Flortaucipir, RO948, Genentech Tau Probe (GTP) 1, or PI-2620 Tau PET standardized uptake value ratio (SUVR).

26. The method of any one of claims 19 to 25, which comprises generating an AD risk score from the dataset that predicts the subject's full brain tau load.

27. The method of any one of claims 19 to 26, which comprises generating an AD risk score from the dataset that predicts whether the subject is likely to have a volume of interest (VOI)-based tau standardized uptake value ratio (SUVR) above a cutoff value.

28. The method of any one of claims 11 to 27, which comprises generating an AD risk score from the dataset that predicts brain neurodegeneration in the subject.

29. The method of claim 28, which comprises generating an AD risk score from the dataset that predicts whether the subject is likely to have a clinical dementia rating indicative of brain neurodegeneration, optionally wherein the AD risk score is a generated using a Light GBM-based algorithm.

30. The method of claim 29, which comprises generating an AD risk score from the dataset that predicts whether the subject is likely to have a clinical dementia rating greater than or equal to 0.5, optionally wherein the AD risk score is a generated using a Light GBM-based algorithm.

31. The method of any one of claims 28 to 30, which comprises generating an AD risk score from the dataset that predicts whether the subject is likely to have a cognitive assessment test score indicative of brain neurodegeneration, optionally wherein the cognitive assessment is the mini-mental state examination (MMSE), Montreal Cognitive Assessment (MOCA), Alzheimer's Disease Assessment Scale-Cognitive section (ADAS-Cog), Delis-Kaplan Executive Function System (D-KEFS) test, or Addenbrookes Cognitive Assessment (ACE-R).

32. The method of any one of claims 28 to 31, which comprises generating an AD risk score from the dataset that predicts whether the subject is likely to have a physical measure of brain neurodegeneration, optionally wherein the physical measure is a reduced cortical thickness, loss of functional connectivity or white matter hyperintensities indicative of brain neurodegeneration.

33. The method of any one of claims 11 to 32, which comprises generating an AD risk score from the dataset that predicts whether the subject is likely to have symptoms sufficient for a diagnosis of mild cognitive impairment or AD, optionally wherein the AD risk score is a generated using a CatBoost-based algorithm.

34. The method of any one of claims 11 to 33, which comprises generating (i) an AD risk score that predicts whether the subject is likely to have an amyloid PET centiloid value of less than a cutoff value, which is optionally 12; (ii) an AD risk score that predicts whether the subject is likely to have an amyloid PET centiloid value greater than or equal to a second cutoff value, which is optionally 21; (iii) an AD risk score that predicts whether the subject is likely to have a Tau PET standardized uptake value ratio (SUVR) in the subject's mesial temporal region which is greater than the 95^{th} percentile SUVR in healthy subjects; (iv) an AD risk score that predicts whether the subject is likely to have a Tau PET standardized uptake value ratio (SUVR) in the subject's temporal region which is greater than the 95^{th} percentile SUVR in healthy subjects; (v) an AD risk score that predicts whether the subject is likely to have a clinical dementia rating greater than or equal to 0.5; and (vi) an AD risk score that predicts whether the subject is likely to have symptoms sufficient for a diagnosis of mild cognitive impairment or AD.

35. The method of any one of claims 7 to 34, which further comprises classifying the subject, based on the subject's AD risk score(s), into one of at least a first risk category and a second risk category, and optionally a third risk category.

36. The method of claim 35, wherein the first risk category indicates that the subject is at a low risk of developing AD.

37. The method of claim 36, which further comprises re-testing the subject for AD in approximately 1-5 years if the subject's AD risk score(s) indicate that the subject is at low risk of developing AD.

38. The method of claim 37, which further comprises re-testing the subject for AD in approximately 3-5 years if the subject's AD risk score(s) indicate that the subject is at low risk of developing AD.

39. The method of any one of claims 35 to 38, wherein the second risk category indicates that the subject has AD or is at elevated risk of developing AD.

40. The method of claim 39, wherein the second risk category indicates that the subject has AD or is at high risk of developing AD.

41. The method of claim 40, which further comprises conducting further testing of the subject for indicators of AD if the subject has an AD risk score(s) indicating that the subject has AD or is at high risk of developing AD, optionally wherein the further testing comprises PET amyloid and/or tau scans, amyloid scanning methods, lumbar puncture amyloid and/or tau procedures, structural MRI, functional MRI, neuroinflammation scanning, diffuse tensor imaging, neuropsychological testing and/or a combination thereof.

42. The method of claim 40 or claim 41, which further comprises generating, in a computerized system, a report recommending administering one or more AD therapeutics to the subject if the subject has an AD risk score(s) indicating that the subject has AD or is at high risk of developing AD.

43. The method of claim 41 or claim 42, which further comprises administering one or more AD therapeutics to the subject if the subject has an AD risk score(s) indicating that the subject has AD or is at high risk of developing AD.

44. The method of claim 42 or claim 43, wherein the one or more AD therapeutics comprise an amyloid disease modifying therapy, a tau therapy, a cholinesterase inhibitor, an NMDA receptor blocker, or a combination thereof.

45. The method of claim 44, wherein the one or more AD therapeutics comprise aducanumab-avwa.

46. The method of any one of claims 41 to 45, which comprises further enrolling the subject in a clinical trial for a candidate AD therapeutic if the subject has an AD risk score(s) indicating that the subject has AD or is at high risk of developing AD.

47. The method of claim 46, which further comprises administering the candidate AD therapeutic to the subject.

48. The method of any one of claims 35 to 47, which comprises classifying the subject, based on the subject's AD risk score(s), into one of at least a first risk category a second risk category, and a third risk category.

49. The method of claim 48, wherein the third risk category indicates that the subject is at moderate risk of developing AD.

50. The method of claim 49, which further comprises re-testing the subject for AD in approximately 1-2 years if the subject's AD risk score(s) indicate that the subject is at moderate risk of developing AD.

51. The method of any one of claims 35 to 50, further comprising generating, in a computerized system, a report comprising a representation of the risk category into which the has been classified.

52. The method of any one of claims 1 to 51, wherein the dataset further comprises the subject's family history of AD.

53. The method of any one of claims 1 to 52, wherein the dataset further comprises the age, gender or education of the subject, or any combination thereof.

54. The method of any one of claims 1 to 53, wherein the data set further comprises one or more genetic risk markers of AD, optionally wherein the one or more genetic risk markers of AD comprise APO E4, Clusterin (CLU), Sortilin-related receptor-1 (SORL1), ATP-binding cassette subfamily A member 7 (ABCA7), or a combination thereof.

55. The method of any one of claims 1 to 54, wherein the step of generating an AD risk score is computer implemented, and wherein the method further comprises providing a notification to the user recommending further testing and/or a neurologic consultation when the subject has an AD risk score(s) indicative of a high risk for developing AD.

56. A method for monitoring the AD status of a subject with one or more AD risk factors, comprising:
(a) performing the method of any one of claims 1 to 55 on one or more fluid samples from the subject and assigning the subject a first AD risk score at a first time point;
(b) performing the method of any one of claims 1 to 55 on one or more fluid samples from the subject and assigning the subject a second AD risk score at a second time point;
(c) comparing the first AD risk score and the second AD risk score to determine if the subject's AD risk score has increased,
thereby monitoring the AD status of the subject.

57. The method of claim 56, which further comprises:
(a) performing the method of any one of claims 1 to 55 on one or more fluid samples from the subject and assigning the subject a third AD risk score at a third time point;
(b) comparing the third AD risk score and the first AD risk score and/or second AD risk score to determine if the subject's AD risk score has increased and/or the rate of change of the subject's AD risk score,
thereby continuing to monitor the AD status of the subject.
(c) if the subject's AD risk score has increased and/or the rate of change of the subject's AD risk score,
thereby continuing to monitor the AD status of the subject.

58. The method of any one of claims 1 to 57, wherein the protein markers comprise at least 5 protein markers.

59. The method of any one of claims 1 to 58, wherein the protein markers comprise one or more tau peptide markers, optionally wherein the one or more tau peptide markers comprise one or more phosphorylated tau peptide markers, optionally wherein the one or more tau peptide markers comprise p-tau 217, p-tau 181, p-tau 231, p-tau 235, or a combination thereof.

60. The method of any one of claims 1 to 59, wherein the protein markers comprise one or more amyloid peptide markers, optionally wherein the one or more amyloid peptide markers comprise Aβ-40, Aβ-42, the ratio of Aβ-40:Aβ-42, the ratio of Aβ-42:Aβ-40, or a combination thereof.

61. The method of any one of claims 1 to 60, wherein the protein markers comprise one or more neurodegeneration markers, optionally wherein the one or more neurodegeneration markers comprise neurofilament light (“NFL”) and/or glial fibrillary acidic protein (“GFAP”).

62. The method of any one of claims 1 to 61, wherein the protein markers comprise one or more metabolic disorder markers, optionally wherein the one or more metabolic disorder markers comprise HbA1c.

63. The method of any one of claims 1 to 62, wherein the protein markers comprise one or more inflammation markers, optionally wherein the one or more inflammation markers comprise C reactive protein (“CRP”), interleukin-6 (“IL-6”), tumor necrosis factor (“TNF”), soluble TREM 2 (“sTREM-2”), a heat shock protein, YKL-40, or a combination thereof.

64. The method of any one of claims 1 to 63, wherein the protein markers further comprise one or more markers other than a tau peptide marker, an amyloid peptide marker, a neurodegeneration marker, a metabolic disease marker, and an inflammation marker (“other markers”), said other markers optionally comprising a proteinopathy marker, e.g., a frontotemporal lobe dementia (FTLD) marker, a Parkinson's Disease marker, a Lewy Body dementia marker, or a combination thereof, optionally wherein the one or more other markers comprise α-synuclein and/or TDP-43.

65. The method of any one of claims 1 to 64, wherein the fluid samples are blood samples, optionally wherein the blood samples are plasma samples.

66. The method of any one of claims 1 to 64, wherein the fluid samples are samples are a combination of blood samples and CSF samples, optionally wherein the blood samples are plasma samples.

67. A method of producing an artificial intelligence-based algorithm for generating an AD risk score for a subject, the method comprising executing, in a computer system having one or more processors coupled to a memory storing one or more computer readable instructions for execution by the one or more processors, the one or more computer readable instructions comprising instructions for:
(a) storing a dataset comprising a plurality of patient records, each patient record comprising quantitative data for at least 4 protein markers in one or more fluid samples from the patient and data for one or more AD surrogate variables for the patient, wherein:
(i) the fluid samples are selected from blood and cerebral spinal fluid (CSF); and/or
(ii) the protein markers comprise at least 3 of a tau peptide marker, an amyloid peptide marker, a neurodegeneration marker, a metabolic disorder marker, and an inflammation marker; and
(b) training a machine learning model with at least a portion of the patient records, wherein the quantitative data for the at least 4 protein markers are input variables and the data for the AD surrogate variable are output variables for the machine learning model, thereby providing an artificial intelligence-based algorithm for generating an AD risk score.

68. The method of claim 67, wherein the artificial intelligence-based algorithm weights the at least 4 protein markers differentially.

69. The method of claim 67 or claim 68, wherein the one or more AD surrogate variables comprise brain amyloid load.

70. The method of claim 69, wherein the patient data for brain amyloid load comprise standardized brain amyloid load data (e.g., PET centiloid data, PET SUVR data, or AmyloidIQ data).

71. The method of claim 69, wherein the patient data for brain amyloid load comprise amyloid PET centiloid data.

72. The method of any one of claims 69 to 71, wherein the at least 4 protein markers comprise one or more tau peptide markers, one or more amyloid peptide markers, one or more neurodegeneration markers, and one or more neuroinflammation markers.

73. The method of claim 72, wherein the one or more tau peptide markers comprise one or more phosphorylated tau peptide markers (e.g., p-tau 181), the one or more amyloid peptide markers comprise Aβ-40, Aβ-42, Aβ-42:Aβ-40 ratio, Aβ-40:Aβ-42 ratio, or a combination thereof, the one or more neurodegeneration markers comprise GFAP, and the one or more neuroinflammation markers comprise sTREM-2.

74. The method of claim 72 or claim 73, wherein the artificial intelligence-based algorithm weights one or more tau peptide markers (e.g., p-tau 181) greater than one or more amyloid peptide markers (e.g., Aβ-42:Aβ-40 ratio), and wherein the artificial intelligence-based algorithm weights one or more amyloid peptide markers (e.g., Aβ-42:Aβ-40 ratio) greater than one or more neurodegeneration markers (e.g., GFAP) and one or more neuroinflammation markers (e.g., sTREM-2).

75. The method of any one of claims 67 to 74, wherein the one or more AD surrogate variables comprise brain tau load.

76. The method of claim 75, wherein the patient data for brain tau load comprise standardized brain tau load data (e.g., PET SUVR data or TauIQ data).

77. The method of claim 75, wherein the patient data for brain tau load comprise Tau PET SUVR data.

78. The method of claim 76 or 77, wherein the Tau PET SUVR data comprise Tau PET SUVR data for the mesial temporal region of the brain.

79. The method of claim 77 or claim 78, wherein the Tau PET SUVR data comprise Tau PET SUVR data for the temporal region of the brain.

80. The method of any one of claims 77 to 79, wherein the Tau SUVR data is a MK6240, Flortaucipir, RO948, Genentech Tau Probe (GTP) 1, or PI-2620 Tau PET SUVR data.

81. The method of claim 80, wherein the Tau PET SUVR data is MK6240 Tau PET SUVR data.

82. The method of any one of claims 75 to 81, wherein the at least 4 protein markers comprise one or more tau peptide markers, one or more amyloid peptide markers, one or more neurodegeneration markers and, optionally, one or more proteinopathy markers.

83. The method of claim 82, wherein the one or more tau peptide markers comprise one or more phosphorylated tau peptide markers (e.g., p-tau 181), the one or more amyloid peptide markers comprise Aβ-40, Aβ-42, Aβ-42:Aβ-40 ratio, Aβ-40:Aβ-42 ratio, or a combination thereof, the one or more neurodegeneration markers comprise GFAP and/or NFL, and the one or more proteinopathy markers comprise TDP43.

84. The method of claim 82 or claim 83, wherein the artificial intelligence-based algorithm weights one or more of the one or more tau peptide markers (e.g., p-tau 181) greater than one or more neurodegeneration markers (e.g., GFAP), and wherein the artificial intelligence-based algorithm weights one or more neurodegeneration markers (e.g., GFAP) greater than one or more amyloid peptide markers (e.g., Aβ-42:Aβ-40 ratio).

85. The method of claim 82 or claim 83, wherein the artificial intelligence-based algorithm weights one or more tau peptide markers (e.g., p-tau 181) greater than one or more amyloid peptide markers (e.g., Aβ-42:Aβ-40 ratio), and wherein the artificial intelligence-based algorithm weights one or more amyloid peptide markers (e.g., Aβ-42:Aβ-40 ratio) greater than one or more neurodegeneration markers (e.g., GFAP).

86. The method of any one of claims 67 to 85, wherein the one or more AD surrogate variables comprise brain neurodegeneration.

87. The method of claim 86, wherein the patient data for brain neurodegeneration comprise clinical dementia rating data.

88. The method of claim 86 or claim 87, wherein the at least 4 protein markers comprise one or more tau peptide markers, one or more amyloid peptide markers, one or more neurodegeneration markers, and one or more inflammation markers.

89. The method of claim 88, wherein the one or more tau peptide markers comprise one or more phosphorylated tau peptide markers (e.g., p-tau 181), the one or more amyloid peptide markers comprise Aβ-40, Aβ-42, Aβ-42:Aβ-40 ratio, Aβ-40:Aβ-42 ratio, or a combination thereof, the one or more neurodegeneration markers comprise GFAP, and the one or more inflammation markers comprise sTREM-2.

90. The method of claim 88 or claim 89, wherein the artificial intelligence-based algorithm weights one or more tau peptide markers (e.g., p-tau 181) greater than one or more neurodegeneration markers (e.g., GFAP), and wherein the artificial intelligence-based algorithm weights one or more neurodegeneration markers (e.g., GFAP) greater than one or more amyloid markers (e.g., Aβ-42:Aβ-40 ratio) and one or more inflammation markers (e.g., sTREM-2).

91. The method of claim 88 or claim 89, wherein the artificial intelligence-based algorithm weights one or more neurodegeneration markers (e.g., GFAP) greater than one or more inflammation markers (e.g., sTREM-2), and wherein the artificial intelligence-based algorithm weights one or more inflammation markers (e.g., sTREM-2) greater than one or more tau peptide markers (e.g., p-tau 181) one or more amyloid peptide markers (e.g., Aβ-42:Aβ-40 ratio).

92. The method of any one of claims 67 to 91, wherein the one or more AD surrogate variables comprise clinical diagnosis of mild-cognitive impairment or AD.

93. The method of claim 92, wherein the patient data for clinical diagnosis of mild-cognitive impairment data comprise affirmative or negative diagnosis of mild-cognitive impairment or AD.

94. The method of claim 92 or claim 93, wherein the at least 4 protein markers comprise one or more tau peptide markers, one or more amyloid peptide markers, one or more neurodegeneration markers and, optionally, one or more proteinopathy markers.

95. The method of claim 82, wherein the one or more tau peptide markers comprise one or more phosphorylated tau peptide markers (e.g., p-tau 181), the one or more amyloid peptide markers comprise Aβ-40, Aβ-42, Aβ-42:Aβ-40 ratio, Aβ-40:Aβ-42 ratio, or a combination thereof, the one or more neurodegeneration markers comprise GFAP and/or NFL, and the one or more proteinopathy markers comprise TDP43.

96. The method of claim 94 or claim 95, wherein the artificial intelligence-based algorithm weights one or more tau peptide markers (e.g., p-tau 181) greater than one or more neurodegeneration markers (e.g., GFAP), and wherein the artificial intelligence-based algorithm weights one or more neurodegeneration markers (e.g., GFAP) greater than one or more amyloid peptide markers (e.g., Aβ-42:Aβ-40 ratio).

97. The method of claim 94 or claim 95, wherein the artificial intelligence-based algorithm weights one or more neurodegeneration markers (e.g., GFAP) greater than one or more proteinopathy markers (e.g., TDP43), and wherein the artificial intelligence-based algorithm weights one or more proteinopathy markers (e.g., TDP43) greater than one or more amyloid peptide markers (e.g., Aβ-42:Aβ-40 ratio) and greater than one or more inflammation markers (e.g., NFL).

98. The method of any one of claims 67 to 97, wherein the protein markers comprise the protein markers described in any one of claims 58 to 64.

99. The method of any one of claims 67 to 98, wherein each patient record further comprises the age of the patient, the age of the patient at a tau PET scan, the gender of the patient, the education of the patient, data for one or more genetic risk markers of AD, or a combination thereof.

100. The method of any one of claims 67 to 99, wherein the fluid samples are blood samples, optionally wherein the blood samples are plasma samples.

101. The method of any one of claims 67 to 99, wherein the fluid samples comprise a combination blood samples and CSF samples, optionally wherein the blood samples are plasma samples.

102. The method of any one of claims 67 to 101, wherein the plurality of patient records comprises at least 100, at least 200, at least 300, at least 500, at least 1000, or at least 5000 patient records and/or step (b) comprises training the machine learning model with at least 100, at least 200, at least 300, at least 500, at least 500, at least 1000, or at least 5000 patient records.

103. The method of any one of claims 67 to 102, wherein the machine learning model is a logistic regression model, a light GBM model, a Random Forest model, a CatBoost model, a linear discriminant analysis model, an Adaptive Boosting model, an Extreme Gradient Boosting model, an Extra Trees model, a Naïve-Bayes model, a K-Nearest neighbor model, a Gradient Boosting model, or a Support Vector model.

104. A method for scoring a subject's risk for developing or already having Alzheimer's disease (AD), comprising
(a) receiving a dataset associated with the subject, wherein said dataset comprises quantitative data for at least 4 protein markers in one or more fluid samples from the subject, optionally wherein:
(i) the fluid samples are selected from blood and cerebral spinal fluid (CSF); and/or
(ii) the protein markers comprise at least 3 of a tau peptide marker, an amyloid peptide marker, a neurodegeneration marker, a metabolic disorder marker, and an inflammation marker;
(b) generating an AD risk score from said dataset using an artificial intelligence-based algorithm produced by the method of any one of claims 67 to 103, thereby scoring the subject's risk for developing or already having AD.

105. A computer implemented method for assessing a subject's risk for developing or already having AD, the method comprising executing, in a computer system having one or more processors coupled to a memory storing one or more computer readable instructions for execution by the one or more processors, the one or more computer readable instructions comprising instructions for:
(a) storing a dataset associated with the subject, wherein said dataset comprises quantitative data for at least 4 protein markers in one or more fluid samples from the subject, wherein:
(i) the fluid samples are selected from blood and cerebral spinal fluid (CSF); and/or
(ii) the protein markers comprise at least 3 of a tau peptide marker, an amyloid peptide marker, a neurodegeneration marker, a metabolic disorder marker, and an inflammation marker; and
(b) generating an AD risk score for the subject from the dataset using the artificial intelligence-based algorithm produced by the method of any one of claims 67 to 103, thereby scoring the subject's risk for developing or already having AD.

106. A system configured to generate an AD risk score according to any one of the methods of any one of claims 7 to 66 and 104 to 105.

107. The system of claim 106, which comprises one or more processors coupled to a memory storing one or more computer readable instructions for execution by the one or more processors.

108. The system of claim 107, wherein the one or more computer readable instructions comprise instructions for:
(a) storing a dataset associated with the subject, wherein said dataset comprises quantitative data for at least 4 protein markers in one or more fluid samples from the subject, wherein:
(i) the fluid samples are selected from blood and cerebral spinal fluid (CSF); and/or
(ii) the protein markers comprise at least 3 of a tau peptide marker, an amyloid peptide marker, a neurodegeneration marker, a metabolic marker, and an inflammation marker; and
(b) generating an AD risk score for the subject from the dataset.

109. The system of any one of claims 107 to 108, wherein the computer readable instructions comprise instructions for generating a report for the subject, optionally wherein the report includes the subject's AD risk score(s) and/or one or more recommendations for the subject from the subject's AD risk score(s).

110. The system of any one of claims 107 to 109, wherein the computer readable instructions further comprise instructions for classifying the subject's risk of having or developing AD, optionally wherein the instructions for classifying the subject's risk of having AD comprising instructions for classifying the subject into one of at least a first risk category and a second risk category, and optionally a third risk category for having or developing.

111. A system configured to produce an artificial intelligence-based algorithm for generating an AD risk score according to any one of claims 67 to 103.

112. The system of claim 111, which comprises one or more processors coupled to a memory storing one or more computer readable instructions for execution by the one or more processors.

113. The system of claim 112, wherein the one or more computer readable instructions comprise instructions for:
(a) storing a dataset comprising a plurality of patient records, each patient record comprising quantitative data for at least 4 protein markers in one or more fluid samples from the patient and data for one or more AD surrogate variables for the patient, wherein:
(i) the fluid samples are selected from blood and cerebral spinal fluid (CSF); and/or
(ii) the protein markers comprise at least 3 of a tau peptide marker, an amyloid peptide marker, a neurodegeneration marker, a metabolic disorder marker, and an inflammation marker; and
(b) training a machine learning model with at least a portion of the patient records, wherein the quantitative data for the at least 4 protein markers are input variables and the data for the AD surrogate variable are output variables for the machine learning model.

114. A system for generating an AD risk score for a subject, comprising one or more processors coupled to a memory storing one or more computer readable instructions for execution by the one or more processors, the one or more computer readable instructions comprising instructions for:
(a) storing a dataset comprising a plurality of patient records, each patient record comprising quantitative data for at least 4 protein markers in one or more fluid samples from the patient and data for one or more AD surrogate variables for the patient, wherein:
(i) the fluid samples are selected from blood and cerebral spinal fluid (CSF); and/or
(ii) the protein markers comprise at least 3 of a tau peptide marker, an amyloid peptide marker, a neurodegeneration marker, a metabolic disorder marker, and an inflammation marker;
(b) training a machine learning model with at least a portion of the patient records, wherein the quantitative data for the at least 4 protein markers are input variables and the data for the AD surrogate variable are output variables for the machine learning model to produce an AI-based algorithm for generating an AD risk score;
(c) generating an AD risk score for the subject using the AI-based algorithm;
(d) classifying the subject as having a low risk of developing AD if the AD risk score indicates that the subject is at a low risk of developing AD, having a medium (or moderate) risk of developing AD if the AD risk score indicates that the subject is at medium (or moderate) risk of developing AD, or high risk of having or developing AD if the AD risk score indicates that the subject is at high risk of having or developing AD; and
(e) generating a report comprising a representation of the risk category into which the has been classified and/or a recommendation for the subject based on the subject's classification.

115. A tangible, non-transitory computer-readable media comprising instructions executable by a processor for executing a method according to any one of claims 1 to 105.
